(19) United States
(12) Patent Application Publication
(10) Pub. No.: US 2026/0265803 A1
(43) Pub. Date: Sep. 10, 2026

(54) METHOD OF SYNTHESIS OF TESTOSTERONAN POLYMER AND DERIVATIVES AND USES THEREOF

(71) Applicant: WEKA BIOSCIENCES, LLC, Santa Fe, NM (US)

(72) Inventor: Nigel J. Otto, Santa Fe, NM (US)

(21) Appl. No.: 19/397,784

(22) Filed: Nov. 21, 2025

Related U.S. Application Data
(60) Provisional application No. 63/724,207, filed on Nov. 22, 2024.

(51) Int. Cl. C12P 33/00; B01D 15/38; C12N 1/20; C12N 9/04; C12N 9/10; C12N 9/12; C12N 9/88; C12N 9/90; C12N 15/70; C12N 15/75; C12N 15/78; C12R 1/11; C12R 1/125; C12R 1/19; C12R 1/38

(52) U.S. Cl. CPC C12P 33/00; B01D 15/3804; C12N 1/20; C12N 9/0006; C12N 9/1029; C12N 9/1048; C12N 9/1096; C12N 9/1241; C12N 9/88; C12N 9/90; C12N 15/70; C12N 15/75; C12N 15/78; C12R 2001/11; C12R 2001/125; C12R 2001/19; C12R 2001/38; C12Y 101/01022; C12Y 203/01157; C12Y 206/01016; C12Y 207/07009; C12Y 207/07083; C12Y 401/01035; C12Y 503/01009; C12Y 504/0201

(57) ABSTRACT

Embodiments of the present invention provides recombinant systems, methods, and compositions for the biological production of Testosteronan (Testan), a linear α-(1→4)-linked glucuronic acid-N-acetylglucosamine polysaccharide. Microbial hosts engineered to express full-length CtTS, N-terminal truncation variants (including d64-CtTS), or the Pseudomonas CIPTS homolog synthesize high-molecular-weight Testan (≥800 kDa) with low polydispersity. Structural analysis by ¹H NMR and 2D HSQC demonstrates that all recombinant enzymes produce Testan that is chemically indistinguishable from native polymer. Purification using ultrafiltration and strong-anion exchange chromatography yields highly defined molecular-weight fractions, including monodisperse populations (PDI~1.02). The invention further provides sulfated derivatives of Testan produced via aqueous sulfation methods, as well as Testan and functionalized Testan compositions useful for chromatographic stationary phases, biomaterial scaffolds, and chemically modifiable polymer platforms. These systems provide the first scalable, fermentation-based production methods for Testan and Testan-derived materials.

### CROSS-REFERENCE TO RELATED APPLICATIONS

This application claims priority to and the benefit of U.S. Provisional Application No. 63/724,207, titled “Method of Synthesis of Testosteronan Polymer and Derivatives and Uses Thereof,” filed Nov. 22, 2024, which is incorporated by reference in its entirety.

### STATEMENT REGARDING FEDERALLY SPONSORED RESEARCH OR DEVELOPMENT

This invention was made with government support under Contract No. 2406036 awarded by the National Science Foundation. The government has certain rights in the invention.

### THE NAMES OF PARTIES TO A JOINT RESEARCH AGREEMENT

Not Applicable.

### INCORPORATION BY REFERENCE OF MATERIAL SUBMITTED ON A COMPACT DISC

Not Applicable.

### SEQUENCE LISTING

The instant application contains a Sequence Listing which has been submitted electronically in xml format and is hereby incorporated by reference in its entirety. Said xml copy, created on May 8, 2026, is named Replacement_20251121_Testan_Seqlisting.xml and is 42,550 bytes in size.

### STATEMENT REGARDING PRIOR DISCLOSURES BY THE INVENTOR OR A JOINT INVENTOR

Not Applicable.

### COPYRIGHTED MATERIAL

Not Applicable.

### TECHNICAL FIELD

Embodiments of the present invention relate to glycosaminoglycan (GAG) biosynthesis, recombinant microbial polysaccharide production, enzyme engineering, and functionalized carbohydrate materials. More specifically, one embodiment of the present invention concerns the recombinant synthesis of Testosteronan (“Testan”), an α-(1→4)-linked glucuronic acid-N-acetylglucosamine polymer, the production of Testan derivatives including sulfated Testan, and the use of these Testan polymers in chromatography, biomaterials, pharmaceutical compositions, and industrial bioprocessing.

### BACKGROUND

Glycosaminoglycan (GAG) and GAG-like polysaccharides play important structural and functional roles in biology and are used in biomedical research, biomaterials, diagnostics, and industrial bioprocessing. Fermentation-based production platforms have been successfully developed for certain microbial polysaccharides, such as heparosan and hyaluronic acid, enabling large-scale access to structurally defined carbohydrates. However, these platforms support only a limited subset of GAG-type backbones and do not provide access to polysaccharides with the specific alternating linkage pattern, rigidity, and physicochemical properties characteristic of Testosteronan.

Testosteronan (“Testan”) is a unique microbial polysaccharide composed of the repeating disaccharide unit [→4)-α-D-GlcA-(1→4)-α-D-GlcNAc-(1→]ₙ. In its native state, Testan is synthesized by Comamonas testosteroni through a dual-action glycosyltransferase, Testosteronan synthase (CtTS). Prior work by the present inventors, disclosed in U.S. Pat. No. 10,273,517, the biochemical activity of CtTS and demonstrated that purified CtTS can produce Testan in vitro using activated sugar nucleotide donors. These in vitro synthesis methods, which rely on purified enzyme and exogenous UDP-sugar precursors, represented the first non-extraction-based access to Testan and are incorporated into the present disclosure by reference.

There are no prior teachings that addresses the N-terminal region of CtTS and whether it is required for activity, nor is there a teaching identifying functional CtTS truncation variants. Nor is there a prior disclosure of any homologous enzymes from other species, such as the Pseudomonas CIPTS homolog described herein, nor is there a prior disclosure that such homologs are capable of synthesizing Testan. Disclosed herein is the first disclosure of a recombinant production of high-molecular-weight Testan (≥800 kDa), low-polydispersity Testan (PDI approaching ~1.02), and purification strategies enabling recovery of monodisperse Testan fractions from a recombinant system according to one embodiment of the present invention.

In addition, no prior work has provided structural confirmation (e.g., ¹H NMR or 2D HSQC overlays) demonstrating that recombinant Testan is chemically identical to native Testan or to in vitro-synthesized Testan. No rheological characterization of recombinant Testan has been reported, and no recombinant Testan has been shown to possess the low-viscosity or low-entanglement properties described in an embodiment of the present invention. No prior teaching provides for aqueous sulfation of recombinant Testan or applications of sulfated Testan as functionalized chromatography or biomaterial components according to one embodiment of the present invention.

There remains a significant unmet need for one or more of the following: a fully functional recombinant microbial production system capable of generating Testan in vivo; recombinant Testan with high molecular weight (≥800 kDa) and low polydispersity; engineered CtTS variants, including N-terminal truncations, with retained polymerizing function; Testan synthase homologs, such as CIPTS, with confirmed Testan-forming activity; robust, scalable fermentation and purification workflows; comprehensive structural validation confirming identity across recombinant and native polymers; and defined Testan derivatives, suitable for chromatographic and materials applications, including sulfated Testan.

One or more embodiments of the present invention fulfills one or more of these needs.

An embodiment of the present invention provides the first recombinant microbial platforms capable of producing Testosteronan (Testan), an α-(1→4)-linked GlcA-GlcNAc polysaccharide, at high molecular weight and low polydispersity. Unlike prior in vitro synthesis methods using purified enzyme, and unlike native production from Comamonas testosteroni, the recombinant systems described herein generate Testan directly in a heterologous host cell and enable scalable, fermentation-based production.

In one aspect, some embodiments of the invention provides recombinant microbial systems comprising a nucleic acid encoding a Testan synthase polypeptide expressed under control of a heterologous promoter. The Testan synthase may be a full-length CtTS enzyme, an engineered N-terminal truncation (including deletion (“Δ”) of amino acids at the N-terminus 1-64 (“Δ1-64”) and broader ΔN variants), or a functional homolog such as the Pseudomonas CIPTS enzyme. In certain embodiments, codon-optimized sequences, multi-copy vectors, or chromosomal integrations enhance expression. Recombinant hosts include E. coli, Bacillus, Pseudomonas, and GRAS organisms suitable for large-scale polymer recovery.

An aspect of the present invention further provides the first demonstration that N-terminal truncations of CtTS retain full enzymatic activity. Structural characterization by 1H NMR and 2D HSQC shows that 41-64 CtTS produces Testan that is chemically indistinguishable from Testan produced by full-length CtTS. Similarly, the CIPTS homolog is shown for the first time to synthesize Testan, yielding a polymer that overlays perfectly with CtTS-derived and native Testan in all NMR dimensions.

An embodiment of a recombinant system described herein produces Testan at number-average molecular weights far exceeding those available in the prior art. In certain embodiments, recombinant Testan exhibits number-average molecular weights of ≥800 kDa and up to ≥1,200 kDa, with polydispersity indices below 1.2. Strong-anion exchange (SAX) chromatography further enables separation into highly defined molecular-weight fractions, including fractions with PDI values approaching monodispersity (PDI ~1.02).

An embodiment of the present invention also provides compositions and methods for purifying recombinant Testan from culture media, including clarification, ultrafiltration, diafiltration, and ion-exchange chromatography. The resulting polymers show consistent structural identity, lack detectable β-linkages or alternative GAG contaminants, and exhibit reproducible NMR signatures and proton integral ratios across multiple enzyme sources.

In another aspect, an embodiment of the present invention provides functionalized Testan derivatives, including sulfated Testan generated using aqueous sulfation chemistries. These derivatives retain polymer integrity and exhibit predictable mobility shifts on Stains-All agarose gels. Functionalized Testan compositions described herein are useful as building blocks for chromatographic stationary phases, biomaterial scaffolds, surface coatings, and chemically modifiable polysaccharide platforms.

Collectively, the recombinant platforms, enzyme variants, purification methods, structurally validated polymers, and Testan derivatives described herein establish the first practical and scalable means for producing Testan and Testan-based materials. These advances enable new applications in chromatography, materials science, enzymology, and polymer engineering that were not achievable using native or in vitro Testan alone.

The following discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

One embodiment of the present invention provides methods for the production, functionalization, and application of Testosteronan, a novel glycosaminoglycan polymer and derivatives thereof. Functionalization for example includes sulfation to create heparinoid polymers and covalent attachment for chromatography and biomaterials. Applications span chromatography, biomaterials, and pharmaceuticals, offering scalable, sustainable solutions for biomedical and industrial needs.

One embodiment of the present invention provides for a method for producing Testosteronan and derivatives thereof, comprising: culturing a recombinant microbial system expressing Testosteronan synthase (for example CtTS) and isolating and purifying the Testosteronan polymer.

Another embodiment of the present invention provides for a chromatography matrix comprising a Testosteronan-functionalized substrate.

Yet another embodiment of the present invention provides for a method for sulfating Testosteronan to produce heparinoid polymers, comprising: sulfating Testosteronan via enzymatic or chemical processes to introduce sulfate groups.

Another embodiment of the present invention provides for a heparinoid polymer comprising sulfated Testosteronan with tailored anticoagulant, anti-inflammatory, or anti-angiogenic properties.

Another embodiment of the present invention provides for a method for synthesizing a Testosteronan polymer, comprising: culturing a recombinant host cell expressing Testosteronan synthase (for example CtTS); providing conditions conducive to enzymatic polymerization of UDP-sugar precursors; isolating the Testosteronan polymer characterized by the repeating disaccharide structure [→4)-α-D-glucuronic acid-(1→4)-α-D-N-acetylglucosamine-(1→]n, wherein n is an integer greater than or equal to 1 wherein the recombinant host cell is selected from the group consisting of Bacillus megaterium, Bacillus subtilis, Escherichia coli, and Pseudomonas aeruginosa.

Another embodiment of the present invention provides for a chromatography system, comprising: a stationary phase substrate functionalized with Testosteronan or derivatives thereof, suitable for use in chromatographic separations.

Another embodiment of the present invention provides for a medical device comprising a medical device, comprising: a biocompatible material coated or embedded with Testosteronan to enhance durability and biological compatibility.

Another embodiment of the present invention provides for a scalable production method for Testosteronan comprising: culturing recombinant host cells expressing codon-optimized CtTS; purifying the Testosteronan polymer using ultrafiltration and ion-exchange chromatography; and functionalizing the purified polymer for industrial or biomedical applications.

Another embodiment of the present invention provides for an injectable hydrogel composition, comprising: cross-linked Testosteronan suitable for sustained drug delivery and tissue regeneration for example the Testosteronan polymer is chemically sulfated to mimic the anticoagulant properties of heparin.

Another embodiment of the present invention provides for a method for separating enantiomers in a racemic mixture comprising utilizing a chromatography column wherein the stationary phase is functionalized with Testosteronan to effect chiral separation for example, the Testosteronan is covalently bonded to the stationary phase substrate via suitable chemical linkages.

Another embodiment of the present invention provides for a drug delivery system, comprising: Testosteronan functionalized with therapeutic agents, wherein the polymer facilitates controlled release of the agents upon administration to a subject.

Another embodiment of the present invention provides for recombinant Testan described herein having one or more of the following characteristics: Molecular weight of between about 100 kDa to about 2 mDa or between about 500 kDa to about 1000 kDa for or example above about 800 kDa or greater (unless otherwise specified). In one embodiment, the Polydispersity index is about ≤1.5, often ~1.02-1.20. Further analysis illustrates no detectable β-linkages, No heparosan or HA contamination by NMR. In some embodiments, it was observed consistent 3:10:2 proton integral ratios (acetoxy:aliphatic:anomeric).

These properties distinguish recombinant Testan from its native ~60 kDa counterpart and from other polysaccharides in the literature. The native CtTS enzyme is a large glycosyltransferase responsible for polymerizing GlcA and GlcNAc into Testan. An embodiment of the present invention includes: full-length CtTS (SEQ ID NO: 1), d64-CtTS truncation mutant (SEQ ID NO: 5) or an alternate N-terminus truncation variant. Additional truncated amino acid sequences [as compared to SEQ ID NO: 1 or SEQ ID NO:6) lead to functional enzymes for example N-terminal truncation variant of SEQ ID NO:1 or SEQ ID NO:6 wherein aa 1-31 are deleted, or aa 1-32, 1-50, 1-60, 1-61, 1-62, 1-63, 1-65, 1-66, 1-67, 1-68, 1-69, 1-70, 1-100, or 1-120 are deleted, any CtTS enzyme variant of SEQ ID NO: 1 having ≥80%, ≥85%, ≥90%, ≥95%, ≥98% sequence identity to conserved amino acids in conserved sequence of SEQ ID NO:8 consensus sequence and exhibits Testan polymerizing activity in an environment in which one or more of Testan from SEQ ID NO:5, or SEQ ID NO:7 or native Testan synthase operates.

Surprisingly, residues 1-64 of SEQ ID NO: 1 are shown to be non-essential for enzymatic function of CtTS, as demonstrated by NMR equivalence between products made by full-length and d64-CtTS enzyme (SEQ ID NO:5) (see FIG. 4A-H). It was further determined that residues 1-65 of SEQ ID NO:6 are shown to be non-essential for enzyme function of CIPTS.

CIPTS (Pseudomonas CIP10 Testan Synthase) SEQ ID NO:6, a homologous enzyme with ~63% pairwise identity (pairwise positive BLSM62 ~76%) to CtTS (SEQ ID NO:1 was cloned and expressed in E. coli. Despite significant sequence divergence, d65-CIPTS (SEQ ID NO: 7) produces Testan that is chemically indistinguishable from CtTS-derived Testan by NMR (FIG. 4C), thereby expanding the genus of Testan-synthesizing enzymes. Expression of CtTS (SEQ ID NO:1), CIPTS (SEQ ID NO:6), d64-CtTS (SEQ ID NO:5), or d65-CIPTS (SEQ ID NO: 7) or a sequence with substantial sequence identity thereto (for example a sequence with at least 55%, 60%, 65%, 70%, 75%, 80%, 85%, 90%, 95%, or greater sequence identity) in microbial hosts such as one or more of the following: E. coli BL21 (DE3), E. coli T7 Express, Bacillus megaterium, Bacillus subtilis, Pseudomonas spp, GRAS organisms for endotoxin-free polymer recovery, but not limited thereto.

Examples of expression vectors include T7 promoters (e.g., pET-28a(+) and illustrated in SEQ ID NO:9 & SEQ ID NO: 10 as shown in FIG. 8A-B, lac and arabinose promoters, codon-optimized gene sequences, His-tags, Maltose Binding Protein (MBP-tags), and multi-copy or chromosomal integration systems.

Synthases may be expressed with solubility-enhancing or stability-enhancing tags such as MBP, SUMO, or signal peptides promoting membrane localization. Induction at OD₆₀₀ 0.6-0.8 using IPTG (0.1-1.0 mM) or arabinose (0.05-0.2%) yields high levels of recombinant synthase.

### 1. N-Terminal Truncation Families

In one embodiment of the present invention, a CtTS enzyme variant with deletion between 1-50, 1-60, 1-65, 1-70, 1-80, 1-100, or 1-120 amino acids at the N-terminal portion of the amino acid sequence of SEQ ID NO:1 or CIPTS enzyme variant with deletion between 1-50, 1-60, 1-61, 1-62, 1-63, 1-64, 1-65, 1-66, 1-67, 1-68, 1-69 or 1-70, 1-80, 1-100, or 1-120 amino acid at the N-terminal portion of the amino acid sequence of SEQ ID NO:6 is described wherein the enzyme variant retains at least 50%, 75%, 90%, or 95% of Testan-forming activity.

### 2. Sequence-Identity Variants

In one embodiment of the present invention, one or more Testan synthase with ≥60%, 70%, 80%, 90%, 95%, or 98% identity to SEQ ID NO: 5 or SEQ ID NO:7 are described, including CIPTS.

### 3. Co-Expression Constructs

One embodiment of the present invention includes UDP-sugar biosynthetic enzymes to enhance precursor availability in the expression system for example UDP-glucose dehydrogenase GlmU (UDP-GlcNAc pyrophosphorylase, GlmM, GlmS, Pgi, GalU, Ugd, etc. (See FIG. 1).

Referring now to FIG. 1, a biosynthetic pathway leading to the Testan precursor sugars UDP-N-acetylglucosamine and UDP-glucuronic acid is illustrated. Enzymatic conversion of glucose into the activated sugar donors required for Testan biosynthesis. Along the hexosamine branch (left), glucose-6-phosphate is converted to fructose-6-phosphate by glucose-6-phosphate isomerase (GPI), which is then aminated to glucosamine-6-phosphate by glutamine-fructose-6-phosphate amidotransferase (GFAT/GlmS). Phosphoglucosamine mutase (PGM/GlmM) converts this intermediate to glucosamine-1-phosphate, followed by acetylation via glucosamine-1-phosphate N-acetyltransferase (GNA/Nat/GlmU-AcT) to form N-acetylglucosamine-1-phosphate. Formation of UDP-N-acetylglucosamine is catalyzed by UDP-N-acetylglucosamine 1-phosphate uridylyltransferase (UAP/GlmU-UTase). Along the UDP-glucuronic acid branch (right), glucose-6-phosphate is converted to glucose-1-phosphate by phosphoglucomutase (PGM/pgm), then to UDP-glucose through UDP-glucose-1-phosphate uridylyltransferase (GalU/UGP). Oxidation of UDP-glucose to UDP-glucuronic acid is carried out by UDP-glucose dehydrogenase (Ugd/UgdH/UdhA). Both activated sugars-UDP-N-acetylglucosamine and UDP-glucuronic acid-serve as substrates for Testosteronan Synthase (e.g. CtTS), which polymerizes the alternating α(1→4)-linked GlcA-GlcNAc disaccharide backbone that constitutes Testosteronan or “Testan”.

### 4. Host Engineering

In one embodiment of the present invention, Testan yield is increased via one or more of the following: overexpression of sugar nucleotide pathways, Deletion of competing pathways, Altered secretion systems, fed-batch cultivation strategies.

In one embodiment of the present invention, derivatives of Testan polymer include one or more of the following: sulfated Testan (sTestan), partially sulfated Testan, Testan-functionalized silica or hydrogels.

In one or more embodiment, Testan and its derivatives are useful for: chromatography (HPLC, chiral separations, ion exchange), biomaterial scaffolds, lubricants and rheology modifiers, drug delivery matrices, heparanase inhibition (diagnostic or therapeutic) (See for example WO/2023250170).

One embodiment of the present invention provides for a recombinant microbial system for producing Testosteronan (“Testan”). The system includes (a) a microbial host cell (for example the microbial host cell may be selected from Escherichia coli, Bacillus subtilis, Bacillus megaterium, or Pseudomonas species); (b) a nucleic acid sequence encoding an enzymatically active Testan synthase wherein the nucleotide sequence is defined as: the nucleotide sequence encoding the amino acid sequence of SEQ ID NO: 15, SEQ NO: 1, SEQ ID NO:6 or SEQ ID NO: 8; or a nucleotide sequence encoding the amino acid sequence of SEQ ID NO: 1 wherein the N-terminal amino acids selected from 1-50, 1-60, 1-61, 1-62, 1-63, 1-65, 1-66, 1-67, 1-68, 1-69 or 1-70 of SEQ ID NO:1 are deleted or a nucleotide sequence encoding the amino acid sequence of SEQ ID NO:6 wherein the N-terminal amino acids selected from 1-50, 1-60, 1-61, 1-62, 1-63, 1-64, 1-66, 1-67, 1-68, 1-69 or 1-70 of SEQ ID NO:6 are deleted or a nucleotide sequence encoding the amino acid sequence of SEQ ID NO:5 or an amino acid sequence having at least about 85% sequence identify to SEQ D NO: 5; or a nucleotide sequence encoding the amino acid sequence of SEQ ID NO:7 or an amino acid sequence having at least about 85% sequence identify to SEQ ID NO:7; or (c) an expression construct comprising a heterologous promoter operably linked to one of the nucleic acid sequences of (b). The enzymatically active Testan synthase produced has a Testan synthase polymerizing activity about 50% or greater as compared to the Testan synthase polymerizing activity of SEQ ID NO:5 or SEQ ID NO:1 or SEQ ID NO:7 or SEQ ID NO:6 under similar polymerizing conditions. The Testan synthase variant may have a deletion of amino acids at the N-terminal portion of SEQ ID NO:1 or SEQ ID NO: 6 and retain at least 90%, 80%, 70&, 60% or 50% polymerizing activity as compared to SEQ ID NO:1 or SEQ ID NO:6. As to the nucleic acid encoding the Testan synthase, it may be codon-optimized for expression in the host cell. A promoter for the system may be selected from T7, lac, arabinose, CMV, BioBrick, or constitutive promoters. The nucleic acid may be present on a multi-copy plasmid or integrated into the host genome. The host cell may further express one or more enzymes selected from UDP-glucose dehydrogenase, UDP-GlcNAc pyrophosphorylase, GlmU, GlmM, GlmS, Pgi, GalU, or Ugd. In some situations, the host is a GRAS organism and the Testan polymer recovered is substantially endotoxin-free. The Testan polymer produced by the system may exhibit a polydispersity index less than 1.5. The Testan polymer produced by the system may exhibit a molecular weight between about 800 kDa and 2 mDa for example between about 800 kDa-1200 kDa.

Another embodiment of the present invention provides a method for producing high-molecular-weight Testosteronan (“Testan”), comprising (a) culturing a recombinant host cell expressing a Testan synthase polypeptide of SEQ ID NO:1, SEQ ID NO:5, SEQ ID NO:6, SEQ ID NO:7, SEQ ID NO:8 or SEQ ID NO:15 or a polypeptide having at least 85% sequence identity to SEQ ID NO:1, SEQ ID NO: 5, SEQ ID NO:6 or SEQ ID NO:7; (b) inducing expression of the Testan synthase under a heterologous promoter to produce a Testan polymer; (c) allowing the Testan polymer to accumulate in a culture medium; and (d) isolating the Testan polymer from the culture medium, wherein the isolated Testan polymer has a number-average molecular weight of between about 100 kDa to about 2 mDa. For example, the culturing comprises fed-batch, batch-fed, or continuous fermentation. Also, the host cell may co-express UDP-sugar biosynthetic enzymes to enhance precursor flux. The culture medium may comprise defined minimal media supplemented with glycerol and ammonium chloride. The method may be performed under cGMP-compatible fermentation conditions. The Testan polymer may be purified using ultrafiltration, diafiltration, anion-exchange chromatography, or combinations thereof to produce a purified Testan polymer comprises at least 95% Testan by weight. For example, the purified Testan polymer is between about 800 kDa and about 1200 kDa.

In another embodiment, a Testosteronan (“Testan”) polymer composition comprises a polysaccharide having the repeating structure [→4)-α-D-GlcA-(1→4)-α-D-GlcNAc-(1→]ₙ, wherein n is an integer greater than 0 and wherein the polymer has a number-average molecular weight of between about 100 kDa to about 2 mDa; and wherein the composition is substantially free of β-linkages, heparosan, or hyaluronic acid contaminants. The Testan polymer can further comprise a functionalized Testan derivative selected from sulfated, carboxymethylated, oxidized, or crosslinked Testan. The Testan polymer is sulfated Testan (sTestan) exhibiting inhibitory activity against human heparanase. The Testan polymer is coupled to a chromatographic stationary phase, including silica, polystyrene-divinylbenzene, or polymer-coated beads. Further, the Testan may be incorporated into a opthalmic solution or biomaterial matrix comprising at least one of collagen, gelatin, alginate, hyaluronan, heparosan or PEG hydrogels. The Testan polymer exhibits a kinematic viscosity at least 30-60% lower than heparosan at equal concentration. Further, the Testan polymer can exhibit a molecular weight between about 800 kDa and 1200 kDa. Further, an embodiment of a Testan polymer is not allergenic to a human. Further, a Testan polymer is not degraded by hyaluronidases or heparanases in the mammalian subject. In one embodiment, the Testan polymer composition includes sterile water.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of embodiments of the present invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 illustrates the biosynthetic pathway leading to the activated sugar donors UDP-GlcNAc and UDP-GlcA required for Testan polymerization.

FIG. 2 shows representative structural disaccharide units of hyaluronan, heparosan, and testosteronan, highlighting differences in linkage stereochemistry.

FIG. 3 presents a pairwise amino-acid alignment of the catalytic regions of CtTS (SEQ ID NO:1) and CIPTS (SEQ ID NO:6) with Blosum62-based similarity shading showing conserved motifs (SEQ ID NO:8) required for Testan polymerization.

FIG. 4A-H provides 1H and HSQC NMR comparisons confirming structural identity of recombinant Testan synthesized by CtTS (SEQ ID NO:1), d64-CtTS (SEQ ID NO:5), and d65-CIPTS (SEQ ID NO:7) as compared to in vitro synthesized Testan.

FIG. 5 displays SEC-MALLS analysis of recombinant Testan fractions before and after anion-exchange purification, demonstrating molecular-weight distribution and polydispersity.

FIG. 6 shows an agarose gel image comparing unsulfated and sulfated Testan relative to HA standards, illustrating altered mobility following sulfation.

FIG. 7 shows an agarose gel comparing recombinant Testan polymer produced by CtTS, d64-CtTS, and CIPTS to commercial HA standard molecular-weight ladders.

FIG. 8A-B depicts representative recombinant expression constructs used for Testan synthase production.

FIG. 9 illustrates TBE-PAGE (6%) analysis of Testan samples stained with Alcian Blue.

FIG. 10 illustrates Alcian Blue-stained 6% TBE-PAGE gel comparing Testan production by multiple heterologous Testan synthase homologs expressed in E. coli.

FIG. 11 illustrates a pairwise amino-acid alignment of the catalytic regions of d64-CtTS (SEQ ID NO:5) and d65-CIPTS (SEQ ID NO:7) with Blosum62-based similarity shading CtTS (SEQ ID NO:5) and CIPTS (SEQ ID NO:7), showing conserved motifs required for Testan polymerization in the consensus sequence SEQ ID NO:15.

FIG. 12 illustrates alignment of Functional Testan Synthase Consensus (SEQ ID NO: 8) With Non-Functional Homolog (SEQ ID NO:3) to produce consensus sequence SEQ ID NO: 16.

### DETAILED DESCRIPTION OF EMBODIMENTS OF THE INVENTION

In one embodiment of the present invention, methods for production of Testan at scale in a recombinant bacterial system using a GRAS (Generally Regarded As Safe) host organism are provided. Comamonas species are known as ‘sometimes pathogens’ and while infections are rare, there are many safer and metabolically superior ‘microbial factory’ systems in which to produce Testan. Recombinant systems can eliminate the need for difficult separations to remove endotoxins (as a Gram negative microbe, the native host produces endotoxins) and other contaminating molecules for biological studies. Recombinant host cells could be Gram positive such as Bacillus, Lactobacillus, Streptomyces, and Corynebacterium species, but may also be Gram negative species such as E. coli, Pseudomonas etc.

Heparosan, built from the identical precursors as Testan, has been produced using such recombinant systems, including Bacillus subtilis, B. megaterium, and E. coli. Heparosan yields from these systems have been robust (5-20 gram/liter). Systems with synthesized codon-optimized versions of CtTS and homologs described herein for Testan production will provide for and enable a metabolically optimized recombinant production system. In general, the goal of polysaccharide production optimization is increasing flux towards target generation with consideration of costs and purity.

Testosteronan's inherent stability and ability to be functionalized make it suitable for a plurality of utilities such as Chromatographic Materials for example Stationary phases for chiral and liquid chromatography; biomedical materials, for example as scaffolds for tissue engineering, drug delivery systems, and wound healing.

For bulk Testan production, three optimization stages are examined:
1) microbial construct design;
2) fermentation, and fidelity of the synthesized Testan polysaccharide; and
3) purification.

Several host organisms comprising codon-optimized CtTS genes driven by various promoters are evaluated at small scale to produce the target polymer. The sugar-polymer product is monitored for size and abundance in the spent media.

A series of parameters is scanned while examining both the CtTS-expressing genes and the host sugar-metabolism pathways in various bacterial culture media, monitoring growth, Testan yields, and contaminant profiles. Various purification modalities for isolating Testan are evaluated, beginning with methods employed for other polysaccharides using industrially scalable technologies. A screening methodology evaluates multiple strains and isolates those that demonstrate superior Testan-production capabilities. Upon identification of productive strains, the process proceeds to upscaling production using fermentation.

The fermentation parameters—namely pH, temperature, oxygen content, and nutrient availability—are systematically optimized to ensure maximal yield and to maintain the molecular fidelity of the synthesized Testan polysaccharides. Post-fermentation, the polysaccharides undergo purification processes. Subsequent characterization is conducted using various analytical techniques to ensure structural and functional consistency.

In one embodiment of the present invention, in vitro chemoenzymatic polymer production method is envisioned. For example a method of producing a polymer, at least a portion of which has the repeat structure [4-D-glucuronic acid-α1,4-D-N-acetylglucosamine-α1-]ₙ ([-4-D-GlcUA-α1,4-D-GlcNAc-α1-]ₙ), the method comprising the steps of culturing a recombinant host cell that comprises a nucleic acid encoding an enzymatically active Testosteronan synthase, wherein the Testosteronan synthase is a single protein that is a dual-action catalyst that utilizes UDP-GlcUA and UDP-GlcNAc to synthesize a polymer having the repeat structure [-4-D-GlcUA-α1,4-D-GlcNAc-α1-]ₙ, wherein the recombinant host cell is cultured under conditions that allow for the production of the enzymatically active testosteronan synthase and under conditions that allow the Testosteronan synthase to produce the polymer having the repeat structure [-4-D-GlcUA-α1,4-D-GlcNAc-α1-]ₙ, wherein the recombinant host cell is cultured in the presence of at least one UDP-sugar or comprises nucleic acids encoding enzymes which catalyze the synthesis of at least one UDP-sugar, and wherein the nucleotide sequence encoding the enzymatically active Testosteronan synthase is defined as:
(a) the nucleotide sequence as disclosed herein for a Testosteronan synthase that produces a polymer having the repeat structure [-4-D-GlcUA-α1,4-D-GlcNAc-α1-]ₙ;
(b) a nucleotide sequence encoding the amino acid sequence of (a);
(c) a nucleotide sequence encoding an amino acid sequence having up to 30 amino acid insertions, deletions, and/or substitutions when compared to (a); and/or
(d) a nucleotide sequence encoding an amino acid sequence having up to 25 amino acid insertions, deletions, and/or substitutions when compared to (a); and isolating the polymer having the repeat structure [-4-D-GlcUA-α1,4-D-GlcNAc-α1-]ₙ.

Heterologous expression systems for other GAGs have been known to produce a variety of polysaccharide chain sizes. The sizes are the result of a combination of synthase-specific phenomena and the biosynthetic pathways that produce the sugar precursors (the fuel for the enzyme). One aspect of the present invention provides for Testan production in the various desirable molecular weight ranges (~10, ~50, 100 kDa, 800-1200 kDa) which may yield different half-lifes and pharmacokinetic behavior with some weight ranges, for example larger polymers, better suited for chromatography matrices).

Preliminary data obtained using native microbial and enzyme-based methods indicate that the molecular weight of Testan can be customized. The native bacteria, as well as the selected recombinant host organisms, grow rapidly in chemically defined synthetic media; accordingly, complex natural nutrient extracts, which are costly and contain numerous organic molecules requiring removal, are not required.

For purification, pilot batches of Testan are protein-free and appear relatively homogeneous.

In one embodiment of the present invention, cGMP (current Good Manufacturing Practices) that are regulatory-approved and used by biotech and pharma will be employed for production of Testan as described herein.

One aspect of the present invention is to increase the flux toward target polysaccharide generation, considering purity and parameters such as yield and molecular weight, through systematic exploration of microbial constructs, fermentation conditions, and purification methods.

Optimizing microbial construct design takes advantage of the genetic architecture of microbial hosts to enhance Testan production. A diverse set of microbial hosts is selected for experimentation. The GRAS organisms Bacillus megaterium and Bacillus subtilis are widely used in the biomanufacturing field and are well known for their safety and robust expression of heterologous genes. Both microbes have been successfully utilized to produce related GAG polysaccharides, including heparosan, hyaluronic acid, and chondroitin. Accordingly, representative strains from these two species serve as initial candidate hosts for evaluation; however, the invention is not limited to these species, as other suitable organisms will be appreciated by those skilled in the art.

Into these host organisms, the CtTS gene is introduced under the control of different promoters. Codon-optimized sequences for the CtTS gene have been generated and are synthesized for both B. megaterium and B. subtilis. These constructs are initially tested using commercially available T7 RNA polymerase-dependent expression systems suitable for both species. Once suitable microbes are identified, additional promoter sequences from the Bacillus BioBrick toolbox are evaluated, and the CtTS construct is integrated into the selected Bacillus genome. As a backup promoter system, moderate-strength lac operon-based systems are also explored, as strong T7 expression can cause metabolic perturbations that reduce yields for certain targets. Examples of codon-optimized sequences, without limitation, follow:

```
LOCUS CtTS_Bacillus_megaterium_optimized 1923 bp DNA linear UNA 19-SEP-2024
DEFINITION Codon optimized CtTS. SEQ ID NO: 11]
ACCESSION urn.local.nucleus.b6ce8f91-882d-42de-9da6-9cb83c4cb81f
VERSION urn.local.nucleus.b6ce8f91-882d-42de-9da6-9cb83c4cb81f
KEYWORDS .
SOURCE Bacillus megaterium
ORGANISM Bacillus megaterium.
FEATURES Location/Qualifiers
ORIGIN
      1 atgtcaggca tgttcaaggt cgctaacgac ttcttctcta atggaaactt cgaaaaagcg
    61 atagagcgat atgaagaaat aatatttaag tacccaggtc ttacggagtt tgcttcaggt
  121 aacttggcgc ttgcacgtcg caaacttggg gaaagacagg aaaacaagag taagtcattg
  181 gtgaacgcca gcaagatctc agagagcatc ttcgtgggga tagccgctat accagagcgt
  241 gcgaaagcgc ttgaaaagac tattgaaagc ttacttccgc aggtggagaa gataggtgtc
  301 tacttaaatg ggtggaagga agtaccagac taccttaaga atgagaaaat ccttgttgaa
  361 gggtttggta aagaagattt gggagatgtc ggtaaatttt tctgggtgga tcagcatgac
  421 ggaatctact ttagctgcga cgacgacctt atctatccaa aggattacgt ggatcgaacc
  481 gtagaaaagc ttaaagagaa aaattacaaa gctgccattg gctggcatgg tagcttattg
  541 cgcgacaatt tctcaaccta ctatgacaag aacagtcgta gagtcttcgt gttttctgct
  601 cacagacctt gggatactcc tgtccacata ttaggcacag gatgttctgc ttttcatact
  661 aaatttttaa agatcaaaaa gagtgacttt ctacacccga acatggcaga catctttttc
  721 agtatcaaag gacaggaaca gaaaatccca ttcattgttt tggcccacga aaaggatgag
  781 attactgaat tcgtcggtgc caaagaaagt agtatctact cacacagtca ggctaacgta
  841 gagagtaaaa aaaatactca cgatctacaa aatgggtttg tgatgaaaaa tatgccttgg
  901 gttatgaatg acgtagaatc actaagtgtt ttaattgtcg gacgtttcga aaactattct
  961 aagggtggta tatacaagtc atgtcacttg attaaggagc acttgtcagc gttgggacat
1021 gatgtcgaca tccatgacac acaaaatccg ttcgcgaagg ctttggaaaa aaaatatgat
1081 ctatgctgga tttatccagg tgatccagag cgtccggact tcagctctgt ggaggataag
1141 atctatgagc taaagagtcg cggcatacca gttattgtta acctttcata cttgtatagc
1201 gaggaccgaa ccatatggat acgcaataag attagagacc ttaacgcaaa agggacaact
1261 ccagtgcttg gggcagtctt taccgagact gctgctaatg accctttgct aaaagatgtg
1321 cgcgattaca tttgtgtcgt tcctaaaacc attctaccaa ccccatgcga gcgatattat
1381 gagttcggtg agagagaagg tatttgttta ggcgacgcca cgaagcttgg aaatgcgaag
1441 gttattgggg gcaatg
LOCUS CtTS_Bacillus_subtilis_optimized 1923 bp DNA linear UNA 19-SEP-2024
DEFINITION Codon optimized CtTS.SEQ ID NO: 12]
ACCESSION urn.local.nucleus.20a82671-35b0-4b19-acb2-1ce8f8fa27bf
VERSION urn.local.nucleus.20a82671-35b0-4b19-acb2-1ce8f8fa27bf
KEYWORDS
SOURCE Bacillus subtilis
ORGANISM Bacillus subtilis.
FEATURES Location/Qualifiers
ORIGIN
      1 atgtcaggca tgtttaaggt agccaacgac ttcttctcaa acgggaactt cgagaaagca
    61 attgaacgtt atgaggaaat catttttaag taccogggac tgacagaatt cgcgtctgga
  121 aacttggctt tagcacgtog gaaacttggt gagcgtcagg aaaataaatc taagagcctg
  181 gttaacgcta gtaaaatctc tgaaagtatc tttgtcggca tagcggcgat accggagcgg
  241 gcgaaagccc tcgaaaaaac gatcgagagt ctcttaccgc aagtcgaaaa aatcggcgtg
  301 tatttgaatg ggtggaaaga agtaccagac tatttaaaaa atgagaagat attggtggaa
  361 ggctttggaa aagaggacct tggggacgtt ggcaaattct tttgggtgga ccagcatgat
  421 gggatctact tctcttgcga cgacgactta atctacccaa aggattacgt tgatcgcact
  481 gtagaaaaat taaaggaaaa gaactacaaa gccgccattg gatggcacgg ttccttgctt
  541 agagataatt ttagtaccta ttatgacaag aactcccgcc gtgtctttgt cttttcagcg
  601 caccgtccgt gggacacgcc tgtgcatata cttggtaccg gctgtagcgc gtttcacacg
  661 aagttcctca agatcaaaaa atctgacttt cttcatccta atatggcgga tatctttttt
  721 tcaatcaagg gccaggagca aaaaattccg ttcattgtgt tggcgcatga gaaagatgag
  781 ataactgagt togtoggcgc aaaagaaagc tcaatatact cacatagcca ggctaatgtg
  841 gaatcaaaaa agaatacgca cgacctccag aatgggtttg tgatgaaaaa catgccatgg
  901 gtaatgaacg acgttgagtc attatccgtt ttaattgttg gtcgtttcga aaattattca
  961 aaaggaggaa tatacaaatc ctgccacctg atcaaagagc acttgagcgc attgggtcac
1021 gatgttgata tacacgatac tcagaatcct ttcgctaagg ccctggagaa aaaatacgac
1081 ctgtgttgga tctacccggg tgatccagaa cggccagatt tcagtagcgt tgaggacaag
1141 atttatgaac tgaaaagtcg ggggatcccg gtaattgtaa acctttcata tttatacagt
1201 gaggaccgta ctatatggat toggaacaag atccgggatc ttaacgcaaa gggcacgact
1261 ccggtgttgg gggcggtatt caccgagact gcggctaacg atccattgtt aaaagatgtc
1321 cgggattaca tatgtgttgt tcctaaaacg attctgccga caccttgtga gcggtactac
1381 gaatttggcg aacgtgaggg catatgcctg ggtgacgcta ctaaacttgg gaatgcaaaa
1441 gtaattggtg gaaacgttaa t
LOCUS CtTS_E. coli_optimized 1923 bp DNA linear UNA 04-AUG-2024
DEFINITION Codon optimized CtTS. SEQ ID NO: 13]
ACCESSION urn.local.nucleus.ee2ecd23-332a-4b81-bd9a-703532643e91
VERSION urn.local.nucleus.ee2ecd23-332a-4b81-bd9a-703532643e91
KEYWORDS .
SOURCE E. coli
ORGANISM E. coli.
FEATURES Location/Qualifiers
ORIGIN
      1 atgagcggta tgtttaaagt ggcaaacgat ttcttttcaa atggcaactt tgagaaggcg
    61 attgaacggt atgaggaaat cattttcaaa tatccaggtc tgacggagtt tgcctcgggg
  121 aatttggccc tggcgcgtcg taaacttggc gagagacaag aaaacaaatc taagagcttg
  181 gttaatgcga gcaagattag cgaatcaatc tttgttggaa ttgccgccat accagagcgg
  241 gcaaaagcct tagaaaagac tattgagtca cttttacctc aagtagaaaa aatcggcgtg
  301 tatttgaacg gttggaagga agtaccagat taccttaaaa atgagaagat attagtcgaa
  361 ggcttcggaa aggaggattt gggggatgtt ggtaaatttt tttgggttga tcagcatgac
  421 ggaatatatt tttcgtgcga cgacgacctg atttacccaa aagactatgt tgaccggact
  481 gttgaaaagt taaaagaaaa gaattataag gcggcgatag gttggcacgg atcactgctt
  541 cgcgacaact tttcgacata ctacgataaa aattcacgga gagtctttgt attcagcgct
  601 catagaccat gggacactcc cgtccacatt cttggaacgg ggtgtagcgc cttccacacg
  661 aaattcttga aaatcaaaaa aagcgacttt cttcacccta atatggcgga tatcttcttt
  721 tcaattaagg gtcaggaaca aaagatccct ttcattgtgc tggcccatga gaaggacgaa
  781 atcacggaat tcgtcggggc caaggaatcg tccatatata gccactctca ggctaacgtt
  841 gagtctaaga aaaatacaca tgatttgcag aacggattcg ttatgaagaa tatgccttgg
  901 gtaatgaatg acgtcgagtc gttaagtgtt ttaattgttg gcagatttga aaactatagc
  961 aagggtggaa tctataaatc gtgtcatctg attaaggaac atttatccgc tctgggacat
1021 gacgttgaca tacacgacac tcagaatcca tttgccaagg ccttggagaa aaagtatgat
1081 ttgtgctgga tctaccctgg agatccggaa cgccccgatt ttagttcagt cgaggacaag
1141 atctacgagc ttaagtcgcg tggtattcct gtcatagtaa atttatcgta cctgtattct
1201 gaggatcgca ccatttggat toggaacaaa attcgcgatc ttaacgccaa gggaacgacg
1261 cctgtcttgg gagcggtctt cacggagact gcggcgaacg atcccctgtt aaaggatgta
1321 agagattata tctgtgttgt ccctaagacg attttaccga ccccctgtga acggtactac
1381 gagtttgggg aaagagaggg aatatgcctt ggtgatgcca ccaaattggg caatgccaaa
1441 gtaatcggcg ggaacgttaa taactggata gacgcgattc acaatcggt
```

The candidate microbial constructs are expanded at small scale using parallel fermentation systems that enable growth of recombinant microbes in data-monitored, scalable formats conducive to optimization of culture conditions. These parallel systems may be configured to operate in various modalities, including chemostat mode (with controlled feeding of a rate-limiting nutrient) and turbidostat mode (maintaining a constant culture density). Real-time optical-density measurements are collected using integrated sensors, permitting calculation of growth rates throughout the experiment. Automated fluid-handling components, such as peristaltic pumps, may be incorporated to enable controlled dosing and removal of media and nutrients. Ports for sampling and air flow are provided, and temperature and agitation are controlled through system software. The size and abundance of the sugar polymer in the spent media post-cultivation are subsequently monitored.

It is important to note that protein expression levels do not necessarily correlate with polysaccharide production. Accordingly, polysaccharide production in the spent media is monitored. Several methods are suitable for assessing polysaccharide levels. Testan production and polymer size are readily visualized using either TBE-PAGE with Alcian Blue staining or agarose gels with Stains-all staining; these methodologies provide both abundance and size information, and approximately 10 to 15 samples can be analyzed in parallel on a single gel to enable direct comparison. Production levels may also be quantitatively determined using the carbazole assay.

The optimal fermentation conditions for maximum Testan yield and minimum contaminants will determine the host/codon/recombinant for testan production based upon user specified criteria such as length of polymer, mw, contamination tolerance or any combination thereof.

The candidate microbial constructs are expanded at small scale using parallel fermentation methods that enable the controlled growth of recombinant microbes in data-monitored, scalable systems suitable for optimizing culture conditions. These small-scale fermentation platforms are capable of operating in various modalities, including chemostat modes (in which a rate-limiting nutrient is fed continuously) and turbidostat modes (in which culture density is maintained at a constant level). Real-time optical-density measurements are collected using integrated sensing modules, allowing continuous calculation of growth rates throughout cultivation. Optional peristaltic-pump integration permits controlled dosing and removal of media and nutrients. Sampling ports and airflow channels facilitate routine sampling, gas exchange, and aeration as required. Temperature, agitation, and other environmental parameters are controlled through an electronic or software interface.

Following cultivation, the size and abundance of the sugar polymer present in the spent media are monitored.

Various bacterial culture media are utilized for the production of Testan. Certain complex bacterial culture media derived from animal or plant extracts may contain polysaccharides that are difficult to separate from the synthesized Testan. To address this potential problem, defined media lacking polysaccharides are evaluated for microbial fermentation. Examples of such media include chemically defined medium (CDM) and Bacto™ CD Supreme Fermentation Production Medium (FPM) from Gibco; however, the invention is not limited thereto.

Microbial growth, Testan yields, and contaminant profiles are continuously monitored. The use of parallel small-scale bioreactor systems enables real-time monitoring of recombinant microbial growth across a series of controlled fermentation conditions. Spent media are sampled throughout the growth cycle and evaluated using electrophoresis and carbazole assays. One aspect of one embodiment of the present invention provides for the establishment of fermentation conditions and media formulations that maximize Testan production while minimizing undesired by-products.

Testan is purified using a purification method, for example as described below. Several established methods are available for the purification of glycosaminoglycans (GAGs) from spent media. The negatively charged polysaccharides are amenable to precipitation using methods similar to those employed for DNA, including precipitation with cetylpyridinium chloride or other cationic detergents. In addition, ion-exchange chromatography, such as purification on HiTrap Q (quaternary ammonium) columns from Cytiva, is effective. As noted elsewhere herein, an ÄKTA FPLC instrument is utilized for such chromatographic purification processes.

Of particular interest are purification methods that are scalable for bulk production. Accordingly, ion-exchange methods employing scalable membrane formats—ranging from approximately 0.5 mL to 1000 L, such as the Sartorius Vivapure ion-exchange membranes—are evaluated. Initial tests using these membrane-based systems with spent media from Comamonas testosteroni cultures have shown promising results. These membranes are reusable for multiple cycles and are capable of sanitation, rendering them suitable for applications within pharmaceutical and biotechnology manufacturing.

The purification methods described herein are applied to polysaccharides produced both by native C. testosteroni and by the recombinant microbial systems described in this patent. The data included in this patent demonstrates successful purification of polysaccharides synthesized by such recombinant constructs, thereby validating the scalability and effectiveness of the disclosed methods.

Methods for Testan isolation are tested and refined in an iterative manner. In consultation with GMP manufacturers, purification strategies are optimized to support highly scalable production of Testan. Purity and yield of Testan following purification are monitored throughout the process.

In addition to the analytical methods already described for assessing Testan purity and yield, the process incorporates assays to ensure that no endotoxins or other contaminants are introduced during production. Suitable assays include limulus-based endotoxin assays, assays for residual host-cell DNA (e.g., PicoGreen), and assays for host-cell proteins.

One aspect of one embodiment of the present invention provides a scalable purification protocol capable of isolating high-purity Testan efficiently while ensuring overall product quality. The methods described herein enable the establishment of a scalable, reproducible, and comprehensive system—from microbial construct through purification—that consistently and cost-effectively produces high-purity Testan, as supported by the data generated and presented in this patent.

In certain other embodiments, the presently disclosed and claimed inventive concept(s) concerns isolated DNA segments and recombinant vectors that include within their sequence a nucleic acid sequence essentially as set forth herein or in SEQ ID NO:2 or a portion thereof or a sequence that codes for any one of SEQ ID NO:1, or SEQ ID NO: 5 for example or a functional Testan synthase as described herein. The term “essentially as set forth in SEQ ID NO: 2” is used in the same sense as described above with respect to the amino acid sequences and means that the nucleic acid sequence substantially corresponds to a portion of SEQ ID NO:2 for example a nucleic acid sequence that codes for SEQ ID NO:5, and has relatively few codons that are not identical, or functionally equivalent, to the codons of SEQ ID NO:2 that codes for SEQ ID NO: 5 and encodes a enzymatically active TS or single-action fragment of TS. The term “functionally equivalent codon” is used herein to refer to codons that encode the same amino acid, such as the six codons for arginine or serine, and also refers to codons that encode biologically equivalent amino acids. The term “Biologically Equivalent Amino Acids” refers to residues that have similar chemical or physical properties that may be easily interchanged for one another (as shown in Table 1).

TABLE 1

| | Conservative and Semi- |
|---|---|
| Amino Acid Group | Conservative Substitutions |
| NonPolar R Groups | Alanine, Valine, Leucine, Isoleucine, |
| | Proline, Methionine, Phenylalanine, |
| | Tryptophan |
| Polar, but uncharged, R Groups | Serine, Threonine, Cysteine, |
| | Asparagine, Glutamine |
| Negatively Charged R Groups | Aspartic Acid, Glutamic Acid |
| Positively Charged R Groups | Lysine, Arginine, Histidine |

It will also be understood that amino acid and nucleic acid sequences may include additional residues, such as additional N- or C-terminal amino acids or 5′ or 3′ nucleic acid sequences, and yet still be essentially as set forth in one of the sequences disclosed herein, so long as the sequence meets the criteria set forth above, including the maintenance of biological protein activity where protein expression and enzymatic activity is concerned. The addition of terminal sequences particularly applies to nucleic acid sequences which may, for example, include various non-coding sequences flanking either of the 5′ or 3′ portions of the coding region or may include various internal sequences, which are known to occur within genes.

Likewise, deletion of certain portions of the TS polypeptide can be desirable. For example, functional truncated versions of pmHAS, the Pasteurella hyaluronan synthase, missing the carboxyl terminus enhances the utility for in vitro use. The truncated pmHAS enzyme is a soluble protein that is easy to purify in contrast to the full-length protein (972 residues). Also, the expression level of the enzyme increases greatly as the membrane is not overloaded.

Allowing for the degeneracy of the genetic code as well as conserved and semi-conserved substitutions, sequences which have between about 40% and about 80%; or more preferably, between about 80% and about 90%; or even more preferably, between about 90% and about 99% of nucleotides which are identical to the nucleotide sequence of SEQ ID NO:2 will be sequences which are “essentially as set forth in SEQ ID NO:2”. In one embodiment, the sequences will be 40%-42% identical, 42%-44% identical, 44%-46% identical, 46%-48% identical, 48%-50% identical, 50%-52% identical, 52%-54% identical, 54%-56% identical, 56%-58% identical, 58%-60% identical, 60%-62% identical, 62%-64% identical, 64%-66% identical, 66%-68% identical, 68%-70% identical, 70%-72% identical, 72%-74% identical, 74%-76% identical, 76%-78% identical, 78%-80% identical, 80%-82% identical, 82%-84% identical, 84%-86% identical, 86%-88% identical, 88%-90% identical, 90%-92% identical, 92%-94% identical, 94%-96% identical, 96%-98% identical, or 98%-100% identical to SEQ ID NO:2. Sequences which are essentially the same as those set forth in SEQ ID NO:2 may also be functionally defined as sequences which are capable of hybridizing to a nucleic acid segment containing the complement of SEQ ID NO:2 under standard or stringent hybridization conditions. Suitable standard hybridization conditions will be well known to those of skill in the art and are clearly set forth herein below. As certain domains and active sites are formed from a relatively small portion of the total polypeptide, these regions of sequence identity or similarity may be present only in portions of the gene. Additionally, sequences which are “essentially as set forth in SEQ ID NO: 1” will include those amino acid sequences that have at least one of the Testosteronan enzyme amino acid motifs (described hereinafter in detail) and that also retain the functionality of an enzymatically active TS or single-action fragment thereof. The polypeptides of the presently disclosed and claimed inventive concept(s) have at least 20%, such as at least 25%, 30%, 40%, 45%, 50%, 55%, 60%, 65%, 70%, 75%, 80%, 90%, or 95% of the TS activity of the full length Testosteronan synthase polypeptide of SEQ ID NO:1 or truncated (d64-CtTS) Testosteronan synthase SEQ ID NO:5. N-terminal truncation families are also within the scope of embodiments of the present invention in that any CtTS variant with deletion of amino acids at the N-terminus as described herein, provided that the enzyme retains at least 50%, 75%, 90%, or 95% of Testan-forming activity. The polypeptides of the presently disclosed and claimed inventive concept(s) have at least 20%, such as at least 25%, 30%, 40%, 45%, 50%, 55%, 60%, 65%, 70%, 75%, 80%, 90%, or 95% of the TS activity of the full length Testosteronan synthase polypeptide of SEQ ID NO:6 or truncated (d64-CtTS) Testosteronan synthase SEQ ID NO:7. N-terminal truncation families are also within the scope of embodiments of the present invention in that any CtTS variant with deletion of amino acids at the N-terminus as described herein, provided that the enzyme retains at least 50%, 75%, 90%, or 95% of Testan-forming activity.

### 2. Sequence-Identity Variants

A Testan synthase with ≥60%, 70%, 80%, 90%, 95%, or 98% sequence identity to SEQ ID NO: 1 or SEQ ID NO: 5 or SEQ ID NO:6 or SEQ ID NO:7, including CIPTS.

As is well known to those of ordinary skill in the art, most of the amino acids in a protein are present to form the “scaffolding” or general environment of the protein. The actual working parts responsible for the specific desired catalysis are usually a series of small domains or motifs. Thus, a pair of enzymes that possess the same or similar motifs would be expected to possess the same or similar catalytic activity, thus they are functionally equivalent. Utility for this hypothetical pair of enzymes may be considered interchangeable unless one member of the pair has a subset of distinct, useful properties. Similarly, certain non-critical motifs or domains may be dissected from the original, naturally occurring protein and function will not be affected; removal of non-critical residues does not perturb the important action of the remaining critical motifs or domains. By analogy, with sufficient planning and knowledge, it is possible to translocate motifs or domains from one enzyme to another polypeptide to confer the new enzyme with desirable characteristics intrinsic to the domain or motif. Such motifs for TS are disclosed in particular hereinafter.

Similarly, certain critical motifs or domains may be changed (mutated) or dissected from the original, naturally occurring protein to thereby affect function; removal of critical residues will perturb the important action of the remaining critical motifs or domains. Such motifs for TS are disclosed in particular hereinafter. The CtTS enzyme in its natural state is a dual action enzyme with two separate active sites or domains. Theoretically, if the sites are relatively functionally independent, then the alteration of one site or domain will not destroy the activity of the other unmutated site. Therefore, mutated, single-action Testosteronan transferases fall within the scope of the presently claimed and disclosed presently disclosed and claimed inventive concept(s).

The term “standard hybridization conditions” as used herein, is used to describe those conditions under which substantially complementary nucleic acid segments will form standard Watson-Crick base-pairing. A number of factors are known that determine the specificity of binding or hybridization, such as pH, temperature, salt concentration, the presence of agents such as formamide and dimethyl sulfoxide, the length of the segments that are hybridizing, and the like. Hybridization may involve the use of shorter nucleic acid segments for hybridization, for example fragments between about 14 and about 100 nucleotides, as well as larger nucleic acid segments, for example, up to the entire length of SEQ ID NO:2. When shorter nucleic acid segments are utilized, exemplary salt and temperature conditions for overnight standard hybridization may include 1.2×-1.8×HPB (High Phosphate Buffer) at 40-50° C. or 5×SSC (Standard Saline Citrate) at 50° C. Washes in low salt (10 mM salt or 0.1×SSC) are used for stringent hybridizations with room temperature incubations of 10-60 minutes. Washes with 0.5× to 1×SSC, 1% Sodium dodecyl sulfate at room temperature are used in lower stringency washes for 15-30 minutes. For all hybridizations, 1×HPB=0.5 M NaCl, 0.1 M Na.sub.2HPO.sub.4, 5 mM EDTA, pH 7.0, and 20×SSC=3 M NaCl, 0.3 M Sodium Citrate with pH 7.0.

For long probes of at least 100 nucleotides in length, stringent conditions are defined as prehybridization and hybridization at 42° C. in 5×SSPE, 0.3% SDS, 200 mg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

The nucleic acid segments of the presently disclosed and claimed inventive concept(s), regardless of the length of the coding sequence itself, may be combined with other DNA sequences, such as promoters, polyadenylation signals, additional restriction enzyme sites, multiple cloning sites, epitope tags, poly histidine regions, other coding segments, and the like, such that their overall length may vary considerably. For example, functional spHAS-(Histidine).sub.6 and ×1HAS1-(Green Fluorescent Protein) fusion proteins have been reported. It is therefore contemplated that a nucleic acid fragment of almost any length may be employed, with the total length preferably being limited by the ease of preparation and use in the intended recombinant DNA protocol.

Another embodiment of the presently disclosed and claimed inventive concept(s) is a recombinant vector comprising at least one of the isolated nucleotide sequences encoding an enzymatically active Testosteronan synthase (“TS”) described herein above. As used herein, the term “recombinant vector” refers to a vector that has been modified to contain a nucleotide sequence (or multiple nucleotide sequences, such as but not limited to, two or more copies of SEQ ID NO:2) that encodes a TS protein, truncated version thereof (for example a nucleic acid sequence that codes for amino acid sequence that is truncated at amino acid position 1 to amino acid position 64 of SEQ ID NO: 1 to produce SEQ ID NO:5 or fragment thereof.

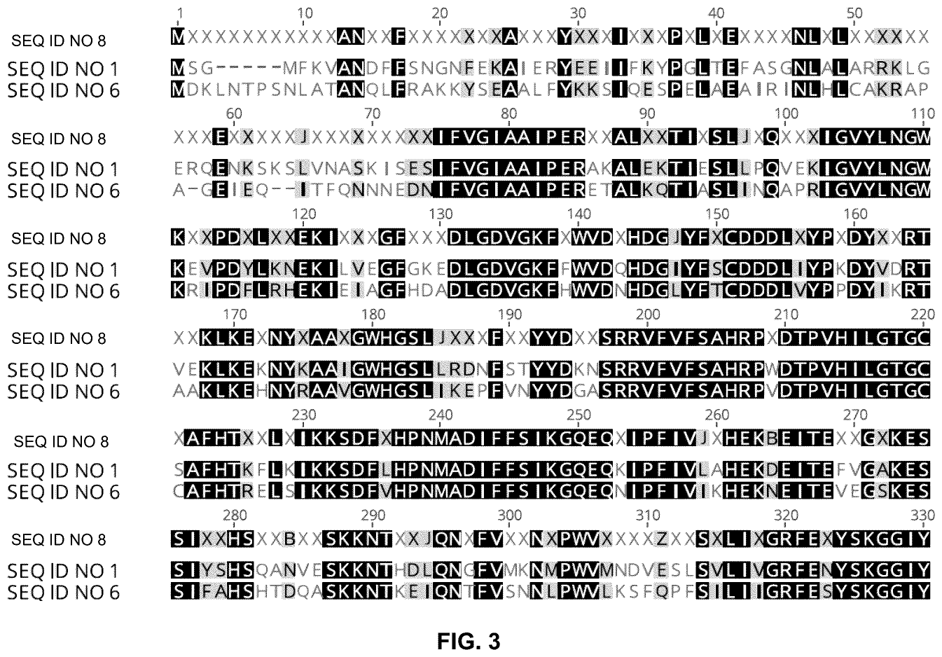

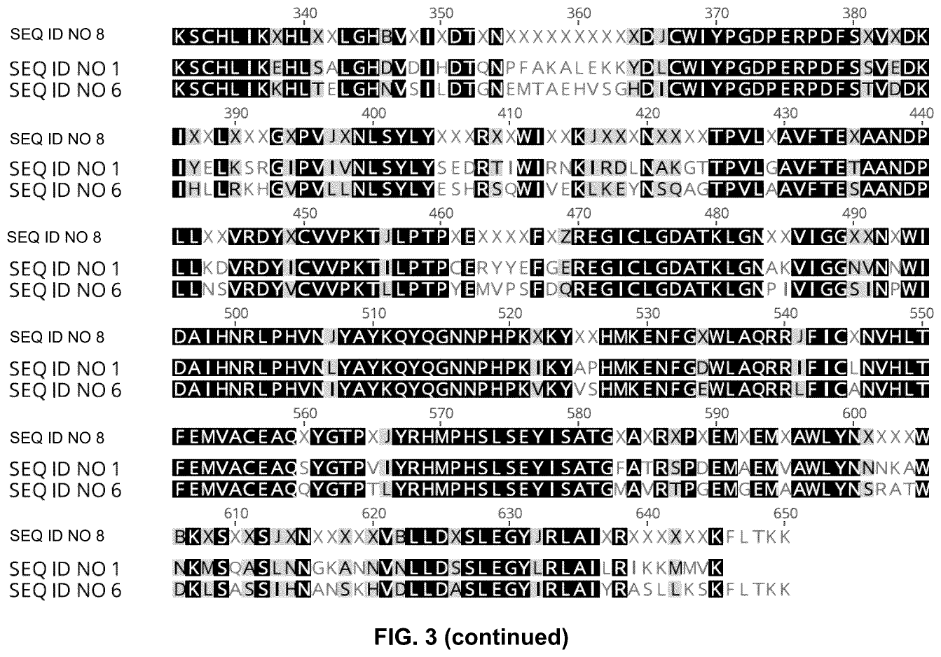

Referring now to FIG. 3 pairwise amino-acid alignment of the full length CtTS (SEQ ID NO:1) and the full length CIPTS (SEQ ID NO:6) with the resulting consensus sequence (SEQ ID NO:8) identified. For sequence alignment shown herein, similarity shading is based on the BLOSUM62 substitution matrix using a threshold value of 1. Columns in which all pairwise residue comparisons meet or exceed this threshold are rendered in black (100% similar). Columns in which the largest similarity group represents 80-100% of residues are shown in dark gray, and columns in which the largest similarity group represents 60-80% of residues are shown in light gray. Residues that do not fall within the identified similarity group remain uncolored. This alignment highlights conserved motifs across the GT45 and GT93 glycosyltransferase domains shared by CtTS and CIPTS and illustrates the sequence features contributing to their shared Testan synthase activity. In the sequences listed herein, “X” represents the conventional one letter symbol for amino acids in Table 5 and represented by A or R or N or D or C or Q or E or G or H or I or L or K or M or F or P or O or S or U or T or W or Y or V, “unknown” or “other”; “B” represents Aspartic Acid or Asparagine; “Z” represents Glutamine or Glutamic Acid; and “J” represents Leucine or Isoleucine.

TABLE 5

| Symbol | 3-Letter Code | Definition |
|---|---|---|
| A | Ala | Alanine |
| R | Arg | Arginine |
| N | Asn | Asparagine |
| D | Asp | Aspartic Acid (Aspartate) |
| C | Cys | Cysteine |
| Q | Gln | Glutamine |
| E | Glu | Glutamic Acid (Glutamate) |
| G | Gly | Glycine |
| H | His | Histidine |
| I | Ile | Isoleucine |
| L | Leu | Leucine |
| K | Lys | Lysine |
| M | Met | Methionine |
| F | Phe | Phenylalanine |
| P | Pro | Proline |
| O | Pyl | Pyrrolysine |
| S | Ser | Serine |
| U | Sec | Selenocysteine |
| T | Thr | Threonine |
| W | Trp | Tryptophan |
| Y | Tyr | Tyrosine |
| V | Val | Valine |
| B | Asx | Aspartic Acid or Asparagine |
| Z | Glx | Glutamine or Glutamic Acid |
| J | Xle | Leucine or Isoleucine |
| X | Xaa | A or R or N or D or C or Q or |
| | | E or G or H or I or L or K or |
| | | M or F or P or O or S or U or |
| | | T or W or Y or V, “unknown” |
| | | or “other” |

SEQ ID NO:5 is the portion of the Testosteronan enzyme CtTS required for polymerization, with the non-essential N-terminal residues of SEQ ID NO: 1 removed. Conserved motifs across the GT45 and GT93 domains-including residues implicated in UDP-sugar binding and glycosyl transfer are indicated as follows with reference to SEQ ID NO:1, amino acids (AA) at position 65-299 represents the GT45 Domain of CtTS; AA at position 300-306 represents the Flexible Linker between GT45 domain and GT93 domain; AA at position 307-640 represents the GT93 Domain and as shown herein, the N-terminal AA 1-64 are dispensible for function and are removed in SEQ ID NO:5 (d64-CtTS) enzyme.

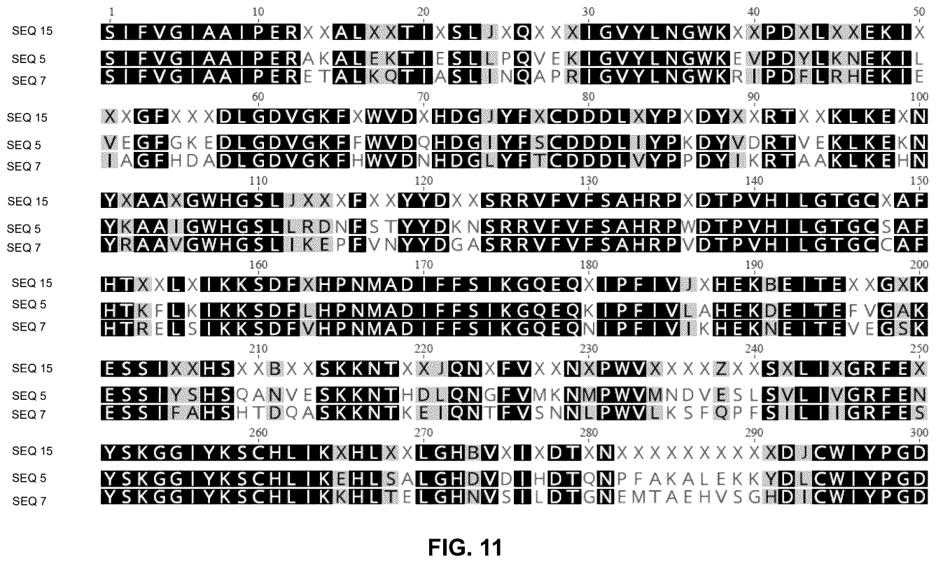

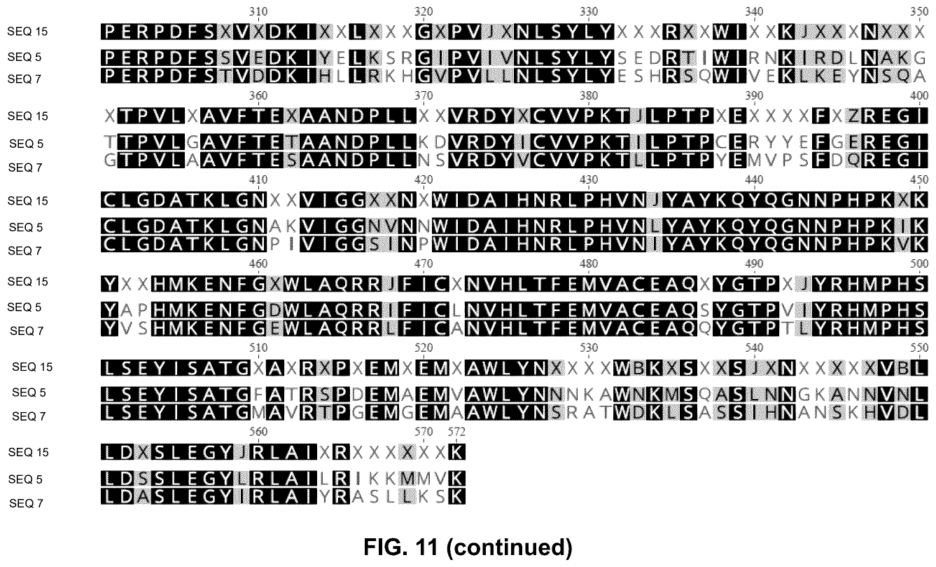

Referring now to FIG. 11, a comparison of SEQ ID NO: 5 and SEQ ID NO: 7 results in a consensus sequence SEQ ID NO:15. The conserved catalytic domain amino acids are identified and the variable amino acids are also identified.

The conservation across the GT45 Domain and the GT93 Domain in Testan synthase explains the observed ability of CIPTS to synthesize Testan identical in structure to CtTS-derived polymer. Protein Statistics: Length (mean): 572 aa (572 codons) Sequences: 2 Identical Sites: 391 (68.4%) Pairwise Identity: 68.4% Pairwise Positive (BLSM62): 81.6%.

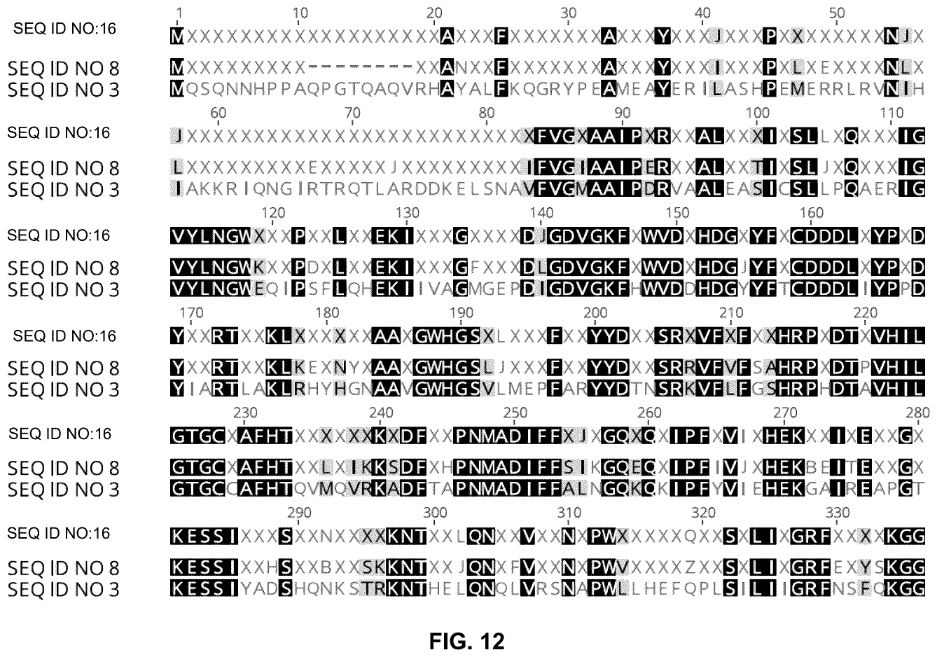

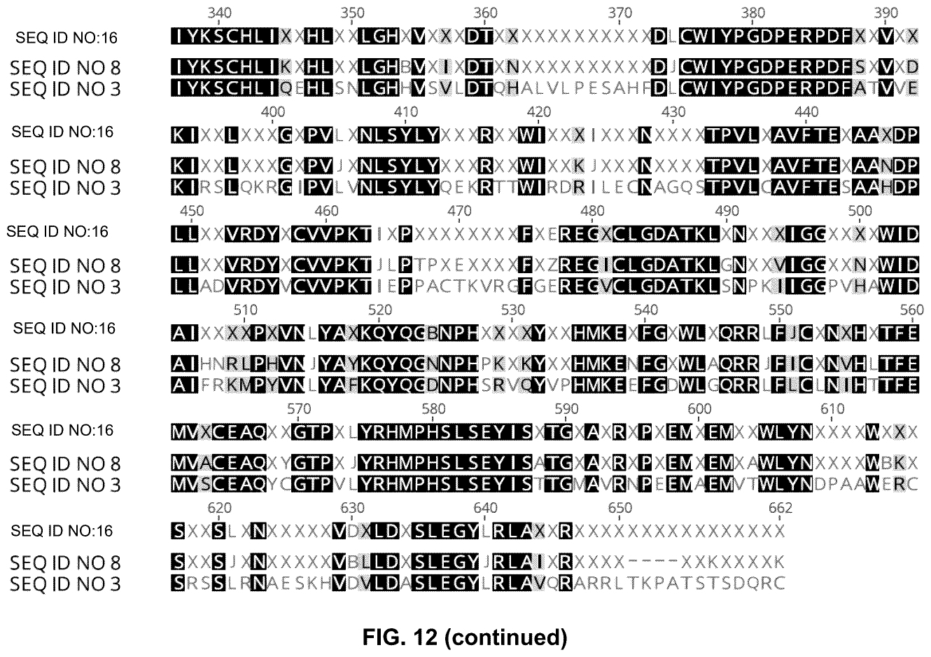

Referring now to FIG. 12, pairwise amino-acid sequence alignment of the functional Testan synthase consensus sequences (SEQ ID NO:8), derived from CtTS and CIPTS, with the non-functional Testan synthase homolog from Candidatus Symbiobacter mobilis (SEQ ID NO: 3). The resulting consensus sequence is SEQ ID NO:16. Similarity shading is based on the BLOSUM62 substitution matrix using a threshold of 1. Columns in which all pairwise comparisons meet or exceed the threshold are rendered in black (100% similar). Columns in which the predominant similarity group represents 80-100% of residues are shown in dark gray, and those representing 60-80% are shown in light gray. Residues not belonging to the similarity group remain uncolored. The alignment highlights that conserved catalytic or substrate coordinating residues and glycosyltransferase motifs present in SEQ ID NO:8 are not conserved in the SEQ ID NO:3 homolog, consistent with the lack of detectable Testan polymerization activity in the latter.

The recombinant vector may be further defined as an expression vector comprising one or more promoters operatively linked to said TS-encoding nucleotide sequence. Examples of vectors that may be utilized in accordance with the presently disclosed and claimed inventive concept(s) include, but are not limited to, plasmids, cosmids, phage, integrated cassettes, virus vectors, combinations thereof, and any other similarly useful vectors known in the art or otherwise contemplated.

A further embodiment of the presently disclosed and claimed inventive concept(s) is a host cell, made recombinant with a recombinant vector as described herein above. The recombinant host cell may be a prokaryotic cell or a eukaryotic cell. As used herein, the term “engineered” or “recombinant” cell is intended to refer to a cell into which a recombinant gene, such as a gene encoding TS, has been introduced. Engineered cells are thus cells having a gene or genes introduced through the hand of man. Recombinantly introduced genes will either be in the form of a cDNA gene, one or more copies of a genomic gene, or will include genes positioned adjacent to a promoter not naturally associated with the particular introduced gene. In certain embodiments, the recombinant host cell may produce Testosteronan.

Where one desires to use a host other than Comomonas, as may be used to produce recombinant Testosteronan synthase, it may be advantageous to employ a prokaryotic system such as E. coli, B. subtilis, Lactococcus sp., or even eukaryotic systems such as yeast or Chinese hamster ovary, African green monkey kidney cells, VERO cells, or the like. For example but not by way of limitation, the host cell may be selected from the group consisting of a Bacillus host such as Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, Bacillus thuringiensis; a Streptomyces host such as Streptomyces lividans or Streptomyces murinus; a gram negative bacteria such as E. coli or Pseudomonas; a fungus or yeast host such as Candida, Kluyveromyces, Pichia, Saccharomyces, Schizosaccharomyces, Yarrowia, Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma. The host cell may also be selected from the group consisting of Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, and Trichoderma viride. Of course, where this is undertaken it will generally be desirable to bring the Testosteronan synthase gene under the control of sequences which are functional in the selected alternative host. The appropriate DNA control sequences, as well as their construction and use, are generally well known in the art as discussed in more detail herein below.

In certain embodiments, the Testosteronan synthase-encoding DNA segments further include DNA sequences, known in the art functionally as origins of replication or “replicons”, which allow replication of contiguous sequences by the particular host. Such origins allow the preparation of extrachromosomally localized and replicating chimeric segments or plasmids, to which TS DNA sequences are ligated. In particular instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

Nucleotide sequences having Testosteronan synthase activity may be isolated by any methods described herein or otherwise contemplated by those of ordinary skill in the art. For example but not by way of limitation, polymerase chain reaction or RT-PCR produced DNA fragments may be obtained which contain full complements of genes or cDNAs from a number of sources, including other strains of Comomonas or from other prokaryotic or eukaryotic sources, such as cDNA libraries. Virtually any molecular cloning approach may be employed for the generation of DNA fragments in accordance with the presently disclosed and claimed inventive concept(s). Thus, the only limitation generally on the particular method employed for DNA isolation is that the isolated nucleotide sequences should encode a biologically functional equivalent TS, and in certain embodiments, the isolated nucleotide sequences should encode an amino acid sequence that contains at least one of the TS amino acid motifs described in detail hereinafter.

Once the DNA has been isolated, it is ligated together with a selected vector. Virtually any cloning vector can be employed to realize advantages in accordance with the presently disclosed and claimed inventive concept(s). Typical useful vectors include plasmids and phages for use in prokaryotic organisms and even viral vectors for use in eukaryotic organisms. Generally Regarded As Safe (GRAS) organisms are advantageous in that one can augment the strain's ability to synthesize Testosteronan through gene dosing (i.e., providing extra copies of the Testosteronan synthase gene by amplification) and/or the inclusion of additional genes to increase the availability of the Testosteronan precursors UDP-GlcUA and UDP-GlcNAc and/or the inclusion of genes that include enzymes that will make modifications (such as sulfation and epimerization) to the Testosteronan polymer. For example, the vector may include (or a separate vector may also be inserted into the recombinant host cell that includes) a nucleic acid segments having a coding region encoding UDP-N-acetylglucosamine pyrophosphorylase and/or an enzymatically active UDP-GlcUA biosynthetic pathway enzyme such as UDP-glucose dehydrogenase and UDP-glucose pyrophosphorylase. The inherent ability of a bacterium to synthesize Testosteronan is also augmented through the formation of extra copies, or amplification, of the plasmid that carries the Testosteronan synthase gene. This amplification can account for up to a 10-fold increase in plasmid copy number and, therefore, the TS gene copy number.

Another procedure that would further augment TS gene copy number is the insertion of multiple copies of the gene into the plasmid. Another technique would include integrating the TS gene into chromosomal DNA. This extra amplification would be especially feasible, since the TS gene size is small. In some scenarios, the chromosomal DNA-ligated vector is employed to transfect the host that is selected for clonal screening purposes such as E. coli or Bacillus, through the use of a vector that is capable of expressing the inserted DNA in the chosen host. In certain instances, especially to confer stability, genes such as the TS gene may be integrated into the chromosome in various positions in an operative fashion. Unlike plasmids, integrated genes do not need selection pressure for maintenance of the recombinant gene.

As described hereinabove, it will also be understood that this presently disclosed and claimed inventive concept(s) is not limited to the particular amino acid sequences [(i.e. SEQ ID NO 1, 5, 6 or 7 for example) and nucleic acid sequences (i.e. SEQ ID NOs: 2, respectively. Recombinant vectors and isolated DNA segments may therefore variously include the TS coding regions themselves, coding regions bearing selected alterations or modifications in the basic coding region, or they may encode larger polypeptides which nevertheless include TS coding regions or may encode biologically functional equivalent proteins or peptides which have variant amino acid sequences.

In certain embodiments, any amino acid changes compared to SEQ ID NO:1, SEQ ID NO:5, SEQ ID NO:6 and SEQ ID NO:7 include amino acid changes that are of a minor nature; particular non-limiting examples include conservative amino acid substitutions that do not significantly affect the folding and/or activity of the protein; small deletions, typically of one to about 30, 40, 50, 60, 70, 80, 90, 100 amino acids or any whole number there between; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

The DNA segments of the presently disclosed and claimed inventive concept(s) encompass biologically functional equivalent TS proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of any techniques known to a person having ordinary skill in the art or otherwise encompassed herein, including but not limited to, site-directed mutagenesis techniques, e.g., to introduce improvements to the enzyme activity or to antigenicity of the TS protein or to test TS mutants in order to examine HS activity at the molecular level.

Also, specific changes to the TS coding sequence may result in the production of Testosteronan having a modified size distribution or structural configuration. One of ordinary skill in the art would appreciate that the TS coding sequence can be manipulated in a manner to produce an altered TS, which in turn is capable of producing Testosteronan having differing polymer sizes and/or functional capabilities. For example, the TS coding sequence may be altered in such a manner that the TS has an altered sugar substrate specificity so that the TS creates a new Testosteronan-like chimeric polymer incorporating a different structure via the inclusion of a previously unincorporated sugar or sugar derivative. This newly incorporated sugar results in a modified Testosteronan having different and unique functional properties. Such mutant GAG synthase proteins have been created in other cases including: (a) a single point mutation that changes the size distribution of the vertebrate HA synthase (Pummill & DeAngelis, J. B. C., 2003) and (b) the chimeric PmHS1/PmHS2 heparosan synthases that utilize a wider variety of UDP-sugar analogs than either natural sequence heparosan synthase (Otto et al, J. B. C. 2012). As will be appreciated by one of ordinary skill in the art given the TS coding sequence, changes and/or substitutions can be made to the TS coding sequence such that these desired properties and/or size modifications can be accomplished.

Basic knowledge on the substrate binding sites (e.g., the UDP-GlcUA site or UDP-GlcNAc site at the DXD motifs, where D-Glu and X=any amino acid); or the oligosaccharide acceptor site of CtTS allows the targeting of residues for mutation to change the catalytic properties of the site. The identities of important catalytic residues of pmHAS, another GAG synthase, have recently been elucidated (Jing & DeAngelis, 2000, the contents of which are expressly incorporated herein in their entirety). Appropriate changes at or near these residues alters UDP-sugar binding. Changes of residues in close proximity should allow other precursors to bind instead of the authentic Testosteronan sugar precursors; thus a new, modified polymer is synthesized. Polymer size changes are caused by differences in the synthase's catalytic efficiency or changes in the acceptor site affinity. Polymer size changes have been made in seHAS and spHAS (U.S. patent application Ser. Nos. 09/559,793 and 09/469,200, the contents of which are expressly incorporated herein by reference) as well as the vertebrate HAS, xIHAS1 (DG42) (Pummill & DeAngelis, 2003, the contents of which are expressly incorporated herein in their entirety) by mutating various residues. Therefore, the presently disclosed and claimed inventive concept(s) encompasses similar or superior versions of mutant CtTS which synthesize modified polymers as well as different sized polymers.

The term “modified structure” as used herein denotes a Testosteronan polymer containing a sugar or derivative not normally found in the naturally occurring Testosteronan polypeptide. The term “modified size distribution” refers to the synthesis of Testosteronan molecules of a size distribution not normally found with the native enzyme; the engineered size could be much smaller or larger than normal.

One of ordinary skill in the art given this disclosure would appreciate that there are several ways in which the size distribution of the Testosteronan polymer made by the TS could be regulated to give different sizes. First, the kinetic control of product size can be altered by environmental factors such as decreasing temperature, decreasing time of enzyme action and/or by decreasing the concentration of one or both sugar nucleotide substrates. Decreasing any or all of these variables will give lower amounts and smaller sizes of Testosteronan product. The disadvantages of these extrinsic approaches are that the yield of product is also decreased, and it is difficult to achieve reproducibility from day to day or batch to batch. Secondly, the intrinsic ability of the enzyme may be altered to synthesize a large or small Testosteronan product. Changes to the protein are engineered by recombinant DNA technology, including substitution, deletion and/or addition of specific amino acids (or even the introduction of prosthetic groups through metabolic processing). Such changes may result in an intrinsically slower enzyme that allows for more reproducible control of Testosteronan size by kinetic means. The final Testosteronan size distribution is determined by certain characteristics of the enzyme that rely on particular amino acids in the sequence. Among the residues absolutely conserved between the now known GAG synthase enzymes, there is a set of amino acids at unique positions that control or greatly influence the size of the polymer that the enzyme can make.

Structurally modified Testosteronan is no different conceptually than altering the size distribution of the Testosteronan product by changing particular amino acids in the desired Testan “TS”. Derivatives of UDP-GlcNAc, in which the acetyl group is missing from the amide (UDP-GlcN) or replaced with another chemically useful group (for example, phenyl to produce UDP-GlcNPhe), is expected to be particularly useful. The free amino group would be available for chemical reactions to derivatize Testosteronan in the former case with GlcN incorporation. In the latter case, GlcNPhe would make the polymer more hydrophobic or prone to making emulsions. The strong substrate specificity may rely on a particular subset of amino acids among the residues that are conserved. Specific changes to one or more of these residues create a functional TS that interacts less specifically with one or more of the substrates than the native enzyme. This altered enzyme then utilizes alternate natural or special sugar nucleotides to incorporate sugar derivatives designed to allow different chemistries to be employed for the following purposes: (I) covalently coupling specific drugs, proteins, or toxins to the structurally modified Testosteronan for general or targeted drug delivery, radiological procedures, etc. (ii) covalently cross linking the Testosteronan itself or to other supports to achieve a gel, or other three dimensional biomaterial with stronger physical properties, and (iii) covalently linking Testosteronan to a surface to create a biocompatible film or monolayer.

Helical polysaccharides are an important class of materials in chiral chromatography due to their ability to discriminate between enantiomers, which are mirror-image forms of chiral molecules. Their unique helical structure and functional diversity make them effective as stationary phases or as part of stationary phases for the separation of enantiomers in analytical and preparative settings.

Testan, a helical polysaccharide composed of repeating α-(1→4)-linked disaccharide units of D-glucuronic acid (GlcA, C₆H₈O₇) and N-acetylglucosamine (GlcNAc, C₈H₁₅NO₆), has useful applications in chromatography for example, chiral chromatography as a versatile chiral stationary phase (CSP) but not limited thereto. Its unique helical structure and the presence of hydrophilic (e.g., hydroxyl and carboxyl) and amide functional groups enable selective interactions with chiral analytes through hydrogen bonding, ionic interactions, and hydrophobic effects. Testan can be utilized either as a coated CSP, where the polysaccharide is physically adsorbed onto silica beads (SiO₂), or as a covalently bonded CSP, where functionalized Testan derivatives are grafted onto the silica surface via silane chemistry, such as using (3-glycidoxypropyl) trimethoxysilane to form covalent bonds. These configurations provide durable and high-resolution CSPs for separating enantiomers in pharmaceutical and chemical industries. For example, the carboxyl groups of GlcA (—COOH) and the acetylamino group of GlcNAc (—NHCOCH₃) can interact asymmetrically with chiral solutes, facilitating enantioselective retention and separation. Testosteronan's natural helical structure provides a chiral environment for selective retention and separation of enantiomers. Tunable Interactions result from functionalization (e.g., sulfation, esterification) of Testosteronan which allows customization of binding strength and selectivity for specific analytes. These tunable interactions are useful in pharmaceutical processing settings when resolution of racemic mixtures into (+) and (−) enantiomers for safer and more effective drug formulations are needed. There is further utility for the use of Testosteronan in chromatography for separation of enantiomers for enhanced sensory properties relating to flavors and fragrances.

In addition to Testosteronan and derivatives thereof being physically adsorbed-effective and easy to manufacture but less durable, Testosteronan and derivatives thereof are capable of being covalently bonded to supports (e.g., via silane chemistry). Testosteronan and derivatives thereof can also be used in Affinity Chromatography with high specificity as functionalized substrate can mimic natural GAGs like heparin, binding proteins and enzymes with high selectivity. Further Testosteronan and derivatives thereof are biocompatible, non-toxic, suitable for biomolecular separations, including therapeutic protein purification. Testosteronan and derivatives thereof can also be used in the isolation of proteins and enzymes for example purification of heparin-binding proteins (e.g., fibroblast growth factors, growth hormones) and isolation of enzymes interacting with glycosaminoglycans for example in production of therapeutic biologics using affinity purification of monoclonal antibodies and cytokines. Functionalization of Testosteronan and derivatives thereof, for example sulfated or epimerized Testosteronan and derivatives thereof enhance binding affinity for target biomolecules.

One embodiment of the present invention provides for a Testan or derivative thereof which are a novel sugar polymer designed specifically to improve chromatography for example chiral chromatography which is useful to separate and analyze mixtures. Chiral chromatography is useful for distinguishing between molecules that are mirror images of each other but behave differently in the body. This capability is especially important in pharmaceutical manufacturing, where the safety and effectiveness of drugs often depend on these molecular distinctions. By creating a stable, synthetic sugar polymer through a microbial process, a more reliable and efficient alternative for chiral chromatography is achieved. This innovation will not only enhance the precision of drug manufacturing but also contribute to safer and more effective medications, aligning with broader public health goals. A chromatography substrate functionalized with Testosteronan may be used for chiral or liquid chromatography. Further a Testosteronan-based chiral stationary phase is useful for separating enantiomers in a racemic mixture.

Another embodiment of the present invention provides for an engineered microbial process to produce a novel sugar polymer that meets the specific demands of chiral chromatography. By optimizing the production of this polymer in microorganisms, the chromatography process seeks to enhance the properties of chromatography materials, such as increased durability and improved ability to separate molecular mirror images. The research will involve refining genetic pathways for maximum yield, scaling up fermentation processes, and establishing purification protocols to ensure that the polymer adheres to high industry standards.

One or more synthetic sugar polymers as described herein are attached to a platform such as a bead to functionalize the platform for chromatography for use in pharmaceutical separation of compounds of interest in chiral chromatography, where its superior performance can significantly improve drug purity and efficacy. Testosteronan is functionalized for covalent or non-covalent attachment to substrates like silica, polymers, and metals. Methods include:

Covalently linking polysaccharides, like Testan, to a support such as attachment to a silica bead utilizes chemistries that ensure stability under chromatographic conditions is beneficial. The choice of chemistry depends on the functional groups present on both the silica surface and the polysaccharide. Below is a list of common attachment chemistries for this purpose.

Functionalization of Polysaccharides: to enhance the utility of polysaccharides such as helical polysaccharides in chiral chromatography, helical polysaccharides are often chemically modified, for example, as to substituents: by introducing functional groups (e.g., phenylcarbamates, benzoyl groups) onto the polysaccharide backbone, the selectivity and strength of interactions can be fine-tuned; as to solubility and stability: Derivatization improves the solubility of the polysaccharides in organic solvents and their thermal stability, making them compatible with high-performance liquid chromatography (HPLC).

Role in Stationary Phases: Helical polysaccharides are typically immobilized onto silica gel or another inert support material to create chiral stationary phases (CSPs). The immobilization methods include: Physical adsorption: The polysaccharide is coated onto the support. Covalent bonding: The polysaccharide is chemically grafted onto the support for greater durability. Column Composition: The resulting columns are packed with these CSPs and used in HPLC systems for enantioselective separations.

Mechanism of Chiral Discrimination: Chiral separation on polysaccharide-based CSPs occurs through differential interactions between the stationary phase and the enantiomers in the mobile phase and include:
1. Interaction Dynamics: As the racemic mixture flows through the column, each enantiomer interacts differently with the helical polysaccharide's chiral environment.
2. Retention Times: The enantiomer that forms stronger or more stable interactions with the stationary phase is retained longer, leading to separation.
3. Factors Affecting Separation: Polarity and size of the analytes; Type and position of substituents on the helical polysaccharide; and Solvent composition and mobile phase conditions; and Temperature

Advantages of Helical Polysaccharides in Chiral Chromatography include one or more of the following: Versatility: Effective for a wide range of chiral compounds, including pharmaceuticals, amino acids, and natural products; High Resolution: Can achieve excellent enantioselectivity and resolution due to the precise chiral environment of the helical structure. •Scalability; Suitable for both analytical and preparative scales, making them valuable in quality control and production.

Examples of Helical Polysaccharides in Use. Amylose Derivatives: •Amylose tris(3,5-dimethylphenylcarbamate) is widely used in CSPs for its high enantioselectivity; Cellulose Derivatives: Cellulose tris(4-methylbenzoate) is another common derivative used in HPLC columns.

Applications for Testan and Testan derivatives for use in chromatography include one or more of the following: Pharmaceuticals: Resolving enantiomers of chiral drugs to ensure efficacy and safety. Agricultural Chemicals: Separating enantiomers of chiral pesticides or herbicides. Natural Products: Isolating pure enantiomers of natural compounds for structural studies and industrial use. Biotechnology: Characterizing chiral intermediates in enzymatic or microbial processes.

Silane-Based Coupling: Silica beads typically have hydroxyl (—OH) groups on their surface, which can react with silane coupling agents. This is one of the most widely used strategies for attaching polysaccharides: Aminosilanes: Reagent: (3-aminopropyl)triethoxysilane (APTES) or (3-aminopropyl) trimethoxysilane. The aminosilane reacts with the silica surface, creating amine (—NH₂) groups that can be further linked to polysaccharides via carbodiimide chemistry or epoxide reactions. Epoxysilanes: Reagent: (3-glycidoxypropyl) trimethoxysilane. The epoxide group on the silane reacts with hydroxyl or amino groups on the polysaccharide, forming stable ether or secondary amine linkages. Isocyanatosilanes: Reagent: (3-isocyanatopropyl)triethoxysilane: The isocyanate reacts with hydroxyl or amine groups on the polysaccharide, forming urethane or urea linkages.

Carbodiimide Chemistry: Carbodiimide-mediated coupling is used to attach carboxyl groups on the polysaccharide to amine-functionalized silica surfaces: Reagent: N-(3-dimethylaminopropyl)-N′-ethylcarbodiimide hydrochloride (EDC), often combined with N-hydroxysuccinimide (NHS) to stabilize the intermediate.

Carboxyl groups (—COOH) on the polysaccharide react with amine groups on the silica to form amide bonds. Periodate Oxidation Followed by Schiff Base Formation. Polysaccharides can be oxidized to generate aldehyde groups, which can then react with amines on the silica surface: Oxidation Reagent: Sodium periodate (NaIO₄) Reaction: Periodate cleaves vicinal diols in the polysaccharide to form aldehydes.

Schiff Base Formation: Reaction: Aldehyde groups react with amines on silanized silica to form imine linkages, which can be stabilized by reduction with sodium cyanoborohydride (NaBH₃CN).

Click chemistry provides a highly specific and efficient method for attaching polysaccharides to silica beads: Azide-Alkyne Cycloaddition: Functionalize the silica surface with azide groups using a silane like (3-azidopropyl)triethoxysilane, and modify the polysaccharide with alkyne groups (e.g., via propargylation). Reaction: Perform a copper(I)-catalyzed azide-alkyne cycloaddition (CuAAC) to form stable triazole linkages.

Thiol-Ene Reaction: Preparation: Introduce thiol groups on the silica surface and alkene groups on the polysaccharide. Reaction: The thiol and alkene react under UV or radical conditions to form thioether bonds.

Epoxide Coupling Epoxide-functionalized silica reacts with nucleophilic groups on the polysaccharide: Reagent: Epichlorohydrin to create epoxide-functionalized polysaccharides or silica surfaces. Reaction: The epoxide reacts with hydroxyl, amine, or carboxyl groups to form ether, secondary amine, or ester linkages.

Succinimidyl Ester Chemistry Polysaccharides with carboxyl groups can be activated to form succinimidyl esters, which react with amine-functionalized silica: Reagent: NHS and EDC to activate carboxyl groups. Reaction: The activated ester reacts with amines on the silica to form amide bonds.

Thiol-Maleimide Coupling Thiol groups on one component can react with maleimide groups on the other: Thiol Modification: Introduce thiol groups on the polysaccharide using reagents like 2-iminothiolane (Traut's reagent). Maleimide Functionalization: Use maleimide-functionalized silanes to prepare the silica surface. Reaction: Thiol and maleimide react to form a stable thioether bond.

### Urethane Linkage Formation

Isocyanate-functionalized silica reacts with hydroxyl or amine groups on the polysaccharide: Reagent: (3-isocyanatopropyl)triethoxysilane, Reaction: Forms urethane (with hydroxyl groups) or urea (with amines) linkages.

Direct Etherification Direct ether bonds can be formed between hydroxyl groups on the polysaccharide and hydroxyl groups on silica: Reagent: Epichlorohydrin or tosyl chloride as a coupling agent. Reaction: Hydroxyl groups on both surfaces are linked via ether bonds under alkaline conditions.

Summary Table 2 of Chemistries

| | | Polysaccharide | Linkage |
|---|---|---|---|
| Chemistry | Silica Functionalization | Functionalization | Type |
| Silane Coupling | Hydroxyl → Silane | Hydroxyl, amine | Covalent (varies) |
| Carbodiimide | Amine-functionalized | Carboxyl | Amide |
| | silica | | |
| Periodate | Amine-functionalized | Aldehyde | Imine (Schiff base) |
| Oxidation | silica | | |
| Click Chemistry | Azide-functionalized | Alkyne or alkene | Triazole, thioether |
| | silica | | |
| Epoxide | Epoxide-functionalized | Hydroxyl, amine | Ether, secondary amine |
| Coupling | silica | | |
| Succinimidyl | Amine-functionalized | Carboxyl (activated | Amide |
| Ester | silica | as NHS ester) | |
| Thiol- | Maleimide-functionalized | Thiol | Thioether |
| Maleimide | silica | | |
| Urethane | Isocyanate-functionalized | Hydroxyl, amine | Urethane, urea |
| Formation | silica | | |
| Direct | Hydroxyl (no prior | Hydroxyl | Ether |
| Etherification | modification needed) | | |

Another embodiment of the presently disclosed and claimed inventive concept(s) is directed to an isolated, enzymatically active Testosteronan synthase encoded by any of the nucleotide sequences described herein above and comprising any of the amino acid sequences described herein above. The Testosteronan synthase is a single protein that is a dual-action catalyst that utilizes UDP-GlcUA and UDP-GlcNAc to synthesize a polymer having the repeat structure [-4-D-GlcUA-α1,4-D-GlcNAc-α1-]ₙ. In certain embodiments, the Testosteronan synthase is produced recombinantly.

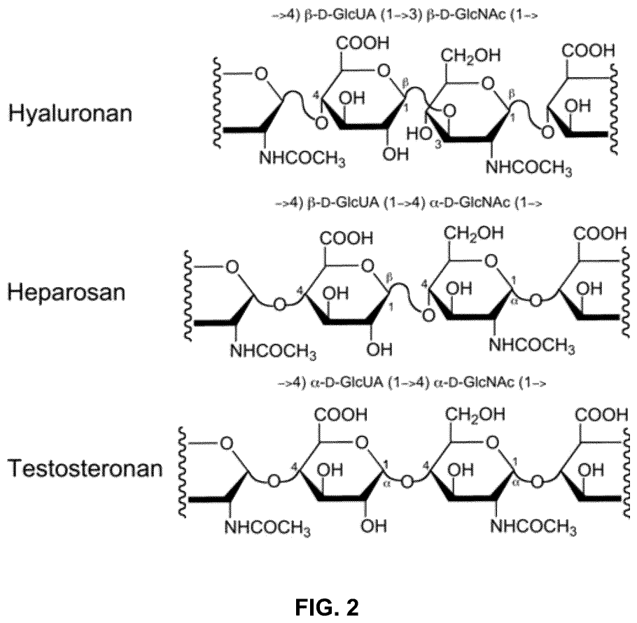

The presently disclosed and claimed inventive concept(s) is also directed to a method of producing a polymer, at least a portion of which has the repeat structure [-4-D-GlcUA-α1,4-D-GlcNAc-α1-]ₙ. In the method, any of the recombinant, enzymatically active Testosteronan synthases described herein above is combined with at least one UDP-sugar (e.g., UDP-GlcUA and/or UDP-GlcNAc) and a functional acceptor that comprises at least one sugar unit. The Testosteronan synthase elongates the functional acceptor to provide a polymer having a structure of at least one of [-4-D-GlcUA-α1,4-D-GlcNAc-α1-]ₙ, GlcUA-α1,4-R and D-GlcNAc-α1-4-R—, wherein R comprises any chemical group. In addition, a heparosan polymer with the general repeat structure of [-4-D-GlcUA-α1,4-D-GlcNAc-α1-]ₙ will also serve as acceptor for CtTS (as shown in FIG. 2 and described in greater detail herein below). Thus, it is possible to create hybrid or chimeric molecules with both types of glycosidic linkages found in heparosan/heparan sulfate/heparin and in Testosteronan in a single polymer molecule. Combining the structures found in both the natural human polymers and the novel polymer will allow new avenues of therapeutic design. For example, regions with altered heparin-binding protein or heparin-modifying enzyme interactions (either stronger or weaker depending on the specific polypeptide) may be embedded in the GAG chain to alter its biologic and therapeutic effects.

In certain additional embodiments, the at least one UDP-sugar may be provided in a stoichiometric ratio to the at least one functional acceptor such that the recombinant Testosteronan synthase elongates the at least one functional acceptor to provide a polysaccharide having a desired size distribution. The resulting polysaccharide may be substantially monodisperse in size and have a polydispersity value in a range of from 1.0 to 1.5. The desired size distribution is obtained by controlling the stoichiometric ratio of UDP-sugar to functional acceptor.

The term “substantially monodisperse in size” as used herein will be understood to refer to defined glycosaminoglycan polymers that have a very narrow size distribution. For example, substantially monodisperse glycosaminoglycan polymers having a molecular weight in a range of from about 3.5 kDa to about 0.5 MDa will have a polydispersity value (i.e., Mw/Mn, where Mw is the average molecular weight and Mn is the number average molecular weight) in a range of from about 1.0 to about 1.1, and preferably in a range from about 1.0 to about 1.05. In yet another example, substantially monodisperse glycosaminoglycan polymers having a molecular weight in a range of from about 0.5 MDa to about 4.5 MDa will have a polydispersity value in a range of from about 1.0 to about 1.5, and preferably in a range from about 1.0 to about 1.2.

The functional acceptor may comprise at least one sugar unit, such as but not limited to, uronic acid and/or a uronic acid analog comprising a substitution at at least one of the C2 and C3 positions thereof. In certain embodiments, the functional acceptor may comprise at least two sugar units, at least one of which is selected from the group consisting of uronic acid (such as but not limited to, GlcUA, iduronic acid (IdoUA) and GalUA), a uronic acid analog comprising a substitution at at least one of the C2 and C3 positions thereof (such as but not limited to, GlcNAcUA, GlcdiNAcUA, and 2-deoxy-2-fluoro-GlcUA), a hexosamine (such as but not limited to, GlcNAc, GalNAc, GlcN and GalN) and a hexosamine analog comprising a substitution at at least one of the C2 and C6 positions thereof (such as but not limited to, GlcN, GlcNAcNAc, GlcN[TFA], GlcNBut, GlcNPro, and 6-F-6-deoxyGlcNAc). Non-limiting examples of functional acceptors that may be utilized in accordance with the presently disclosed and claimed inventive concept(s) include a Testosteronan oligosaccharide, polysaccharide and/or polymer and a heparosan oligosaccharide, polysaccharide and/or polymer.

In one embodiment, the functional acceptor may be a Testosteronan oligosaccharide of about 3 sugar units to about 4 kDa, or a Testosteronan polymer having a mass of about 4 kDa to about 1 MDa. In another embodiment, the functional acceptor may be a heparosan oligosaccharide of about 3 sugar units to about 4 kDa, or a heparosan polymer having a mass of about 4 kDa to about 2 MDa. In another embodiment, the functional acceptor may be a Testosteronan oligosaccharide, polysaccharide or polymer; a heparosan oligosaccharide, polysaccharide or polymer; a heparin oligosaccharide, polysaccharide, or polymer; a heparin oligosaccharide, polysaccharide or polymer; a heparosan-like oligosaccharide, polysaccharide or polymer; or a sulfated or modified oligosaccharide, polysaccharide or polymer, or a GlcUA-based or GlcUA-analog glycoside. In yet another embodiment, the functional acceptor may be an extended acceptor such as Testosteronan chains, heparosan chains, mixed glycosaminoglycan chains, analog containing chains or any combination thereof.

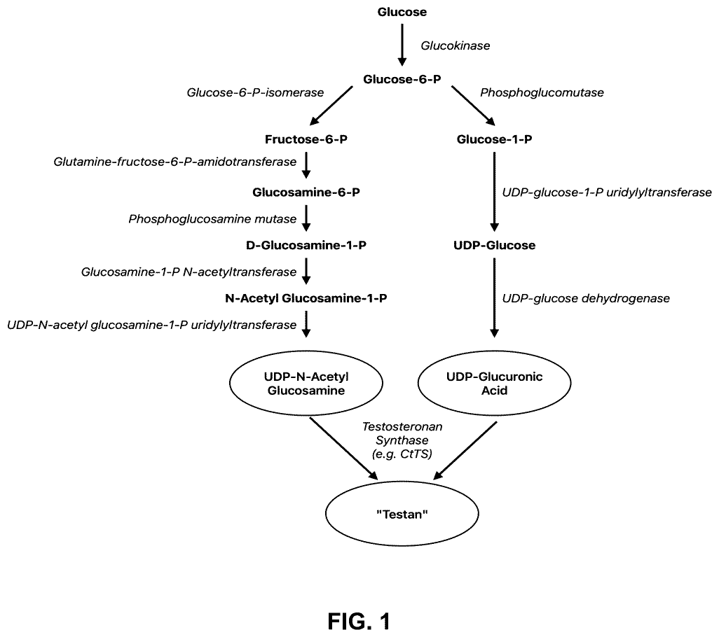

Another functional acceptor class that may be utilized in accordance with the presently disclosed and claimed inventive concept(s) includes synthetic glycosides (i.e., sugars that have a non-sugar component at the reducing end) or similar synthetic carbohydrates. These molecules are less expensive and can possess useful groups. Glucuronic acid and its glycosides, after two step-wise extensions prior to reaction synchronization, are effective acceptors with CtTS (as seen in FIG. 1 and described in further detail herein).

Referring now to FIG. 2, comparison of the repeating disaccharide structures of hyaluronan, heparosan, and testosteronan is illustrated.

Representative structural cartoons showing the linkage stereochemistry and monosaccharide composition of three related glycosaminoglycan (GAG) polysaccharides. Hyaluronan consists of repeating→4) β-D-GlcUA (1→3) β-D-GlcNAc (1→units, forming an alternating β-1,4/β-1,3 linkage pattern. Heparosan, the unsulfated precursor of heparan sulfate and heparin, is composed of repeating→4) β-D-GlcUA (1→4) α-D-GlcNAc (1→disaccharides, distinguished by the α-1,4 linkage to GlcNAc. Testosteronan, the polymer produced by CtTS and related bacterial synthases, contains repeating→4) α-D-GlcUA (1→4) α-D-GlcNAc (1→residues, featuring α-1,4 linkages for both sugar units. Differences in anomeric configuration and glycosidic linkages among these polymers define their distinct conformations, biological properties, and physicochemical behaviors.

The functional acceptor may further include a moiety selected from the group consisting of a fluorescent tag, a radioactive tag or therapeutic, an affinity tag, a detection probe, a medicant, a biologically active agent, a therapeutic agent, and combinations thereof. As a non-limiting example, an artificial para-nitrophenyl moiety was used in the acceptor glycoside; thus, the resulting Testosteronan chains have an attached non-sugar group. In addition, the UDP-sugar and/or UDP-sugar analog may be radioactive or nuclear magnetic resonance-active.

The method may further include the step of providing a divalent metal ion, such as but not limited to, manganese, magnesium, cobalt, nickel and combinations thereof. In addition, the method may be carried out in a buffer having a pH from about 4 to about 9.

A yet further embodiment of the presently disclosed and claimed inventive concept(s) is directed to an additional method of producing a polymer, wherein at least a portion of which has the repeat structure [-4-D-GlcUA-α1,4-D-GlcNAc-α1-]ₙ. In the method, any of the recombinant host cells described herein above is cultured under conditions that allow for the production of a polymer having the repeat structure [-4-D-GlcUA-α1,4-D-GlcNAc-α1-]ₙ. The resultant polymer may further be isolated and/or purified, either from the culture medium or the recombinant host cell. The recombinant host cell may include (either genomically or through the addition of a vector) nucleic acid segments encoding enzymes which produce UDP-GlcUA and UDP-GlcNAc. If the recombinant host cell does not produce the sugar precursors, UDP-GlcUA and UDP-GlcNAc may be supplied to the recombinant host cell.

Another embodiment of the presently disclosed and claimed inventive concept(s) is directed to another method of producing the Testosteronan polymers described herein. In this method, native host cells are cultured under conditions that allow for the production of a polymer having the repeat structure [-4-D-GlcUA-α1,4-D-GlcNAc-α1-]ₙ; the polymer so produced is then isolated from the native host cells. Non-limiting examples of native host cells that may be utilized in accordance with the presently disclosed and claimed inventive concept(s) include Coma monas species, such as but not limited to, Coma monas testosteroni, or Pseudomonas allies, or any other microbe that possesses a functional Testosteronan synthase or homolog or analog. The polymer so produced may further be purified by any methods described herein or otherwise known in the art.

The presently disclosed and claimed inventive concept(s) further includes isolated and/or purified testosteronan polymers (as well as testosteronan-like (testosteronan-based) polymers) produced by any of the methods described herein above. In certain embodiments, the isolated/purified testosteronan polymers may be recombinantly produced and/or substantially monodisperse in size (as described in detail herein above). The isolated/purified testosteronan polymers produced in accordance with the presently disclosed and claimed inventive concept(s) may be substantially insensitive to digestion by a degrading enzyme that acts upon at least one of heparosan, heparin, heparan sulfate, chondroitin and hyaluronan. The isolated/purified testosteronan polymers may also be sulfated. While heparin is a widely used anticoagulant derived from animal tissues, it poses challenges related to contamination risks, variability, and limited supply chains. Additionally, heparin's strong anticoagulant activity limits its broader use in oncology, inflammation control, and tissue engineering. Testosteronan provides a platform for creating microbial, non-animal-derived heparinoid polymers with tunable biological activity and broad applications.

In one embodiment, a Testosteronan polymer is useful as a biodegradable material such as a bioyarn, suture, implant, scaffold for medical use or as a drug delivery device (for example, Testosteronan nanoparticles and films are functionalized for controlled drug release) and as a substitute for heparin, for example, a sulfated Testosteronan polymer for use as a biocompatible anticoagulant. It is thought that Sulfated Testosteronan mimics heparin's interaction with antithrombin but with tunable anticoagulant activity. Sulfated Testosteronan is useful for selective isolation of heparin-binding proteins. Further, a sulfated Testosteronan is also useful as a film for wound healing or drug delivery, for example Sulfated Testosteronan accelerates wound repair by promoting angiogenesis and reducing inflammation. While a cross-linked Testosteronan is useful as a hydrogel for tissue engineering applications. Cross-linked or sulfated Testosteronan hydrogels support cell adhesion, proliferation, and differentiation. Anti-inflammatory Agents Sulfated derivatives bind cytokines to modulate immune responses. Oncology Heparinoid analogs inhibit angiogenesis and tumor metastasis by targeting VEGF and FGF. Therefore, sulfated derivatives of Testosteronan and derivatives thereof are useful for mimicking heparin for anticoagulant, anti-inflammatory, and anti-cancer therapies.

One aspect of one embodiment of the present invention provides for chromatography with Testosteronan and derivatives thereof used to functionalize chromatography substrate provide benefit in charge-based separation: Testosteronan's negatively charged carboxyl groups (from glucuronic acid) provide strong ionic interactions with positively charged analytes. Adjustable Functionalization is possible for example sulfation increases the density of negative charges, improving the resolution of cationic species.

Applications in which the functionalized Testosteronan and derivatives thereof are useful include: Protein Separation: for example Fractionation of proteins by isoelectric points (pI); Enrichment of basic proteins (e.g., histones, lysozymes; Nucleic Acid Purification for example purification of RNA and DNA, particularly in plasmid or viral vector production; polysaccharides and Oligosaccharides, for example separation of charged carbohydrates. When used as a stationary phase: Testosteronan-functionalized beads provide for strong anion exchange. Optional covalent binding for durability in industrial processes.

When Testosteronan and derivatives thereof are used in hydrophilic interaction chromatography (HILIC), Testosteronan's hydroxyl, amide, and carboxyl groups provide strong hydrophilic interactions for a highly polar stationary phase and are effective for analytes in high organic solvent conditions (e.g., acetonitrile). Applications of chromatography substrate functionalized with Testosteronan and derivatives thereof include: carbohydrate and sugar analysis: separation of mono- and oligosaccharides in complex mixtures; Metabolomics: analysis of polar metabolites such as amino acids, nucleotides, and organic acids; pharmaceutical excipients: characterization of excipients and hydrophilic drugs.

Functionalization: Acylated Testosteronan derivatives enhance the phase's polarity and selectivity. size-exclusion chromatography (SEC) tunable pore sizes: polymerization of Testosteronan allows customization of pore dimensions for optimal size-based separations. Biocompatibility: Non-reactive matrix suitable for biomolecular separations. Applications: Protein Aggregation Analysis: Separation of monomers, dimers, and higher-order protein aggregates. Synthetic Polymers: Determination of molecular weight distributions of synthetic or natural polymers. Virus Particle Isolation: Fractionation of viral particles for vaccine or gene therapy development. Configuration: Testosteronan-crosslinked hydrogels or porous beads form the stationary phase. Reverse-Phase Chromatography (RPC) Key Advantages: Hydrophobic Derivatives: Modified Testosteronan (e.g., alkylated or benzylated) interacts with non-polar analytes. Durable Covalent Bonding: Ensures stability under high-pressure and high-temperature conditions. Applications: Pharmaceuticals: Purification of hydrophobic drug intermediates and impurities. Lipids and Hydrophobic Biomolecules: Isolation of lipids, sterols, and hydrophobic peptides. Functionalization: Derivatization with hydrophobic groups improves selectivity for non-polar compounds. Membrane Chromatography Key Advantages: High-Throughput Capability: Suitable for rapid processing of large sample volumes; Scalability: Ideal for industrial-scale purification processes. Applications include: Therapeutic Protein Purification: Capture of monoclonal antibodies, fusion proteins, and vaccines. Virus and Particle Separation: Isolation of virus-like particles (VLPs) and extracellular vesicles. Bioprocessing: High-speed removal of contaminants (e.g., endotoxins, DNA).

Testosteronan and derivatives thereof-functionalized membranes may be integrated into filtration units as a stationary phase; used with supercritical Fluid Chromatography (SFC) that utilizes supercritical CO₂ as the mobile phase, offering a sustainable and efficient alternative to traditional liquid chromatography. Advantages: Green Chemistry: Reduces the use of organic solvents. Enhanced Efficiency: Faster separations due to lower viscosity of supercritical fluids. Applications: Pharmaceuticals: Separation of active pharmaceutical ingredients (APIs). Natural Products: Isolation of plant-derived alkaloids and flavonoids. Simulated Moving Bed Chromatography (SMB) Simulated moving bed chromatography is a continuous process that enables highly efficient separation of compounds. Advantages: High Yield: Minimal loss of target compound. Cost Efficiency: Reduced solvent and stationary phase usage. Applications: Pharmaceutical Intermediates: Large-scale separation of enantiomers. Biofuels: Purification of sugars and organic acids. Role of Testosteronan: Testosteronan-functionalized columns provide improved separation performance due to their stability and selectivity. Preparative Chromatography Preparative chromatography focuses on the large-scale isolation and purification of valuable compounds. Applications: Large-scale isolation of APIs and excipients. Purification of bioactive compounds from natural extracts. Role of Testosteronan: Functionalized Testosteronan provides scalability and reproducibility for industrial-scale separations.

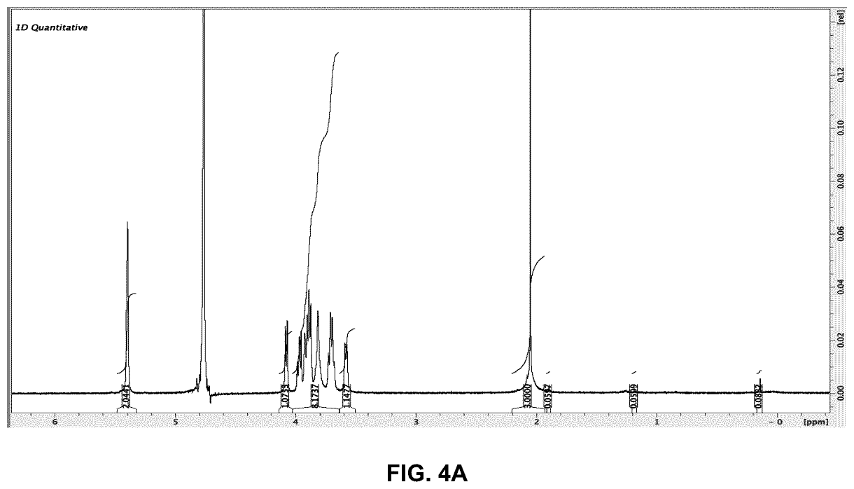

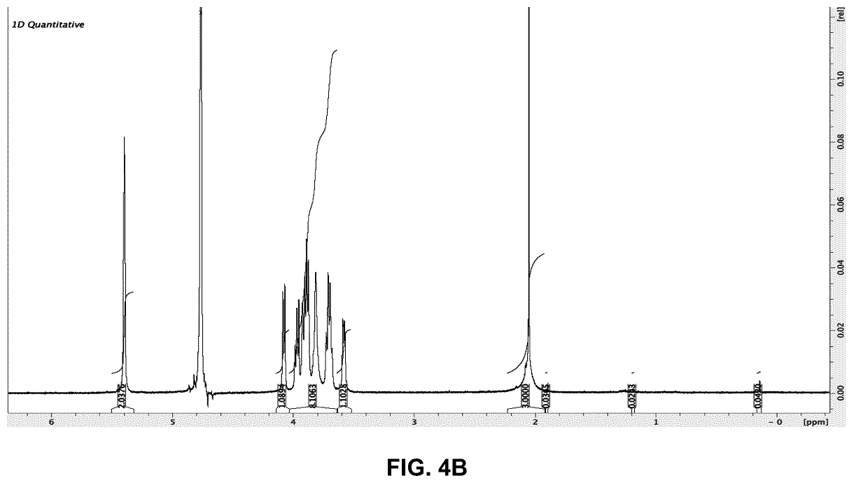

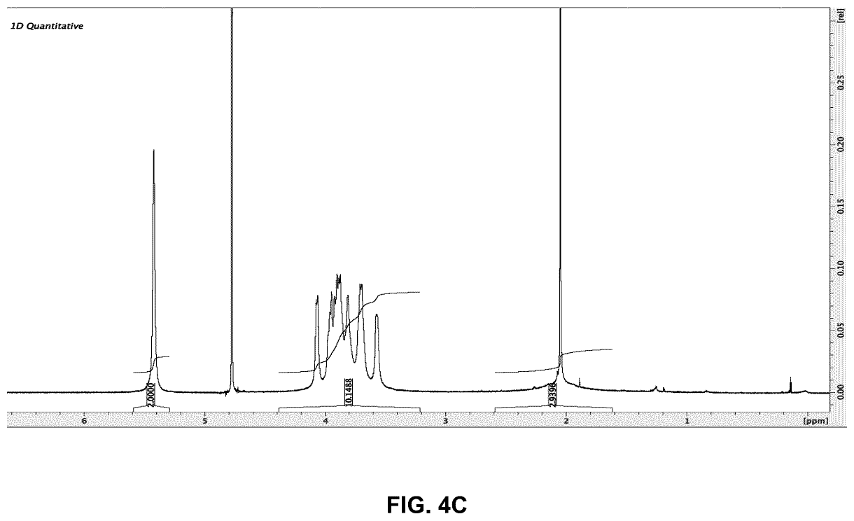

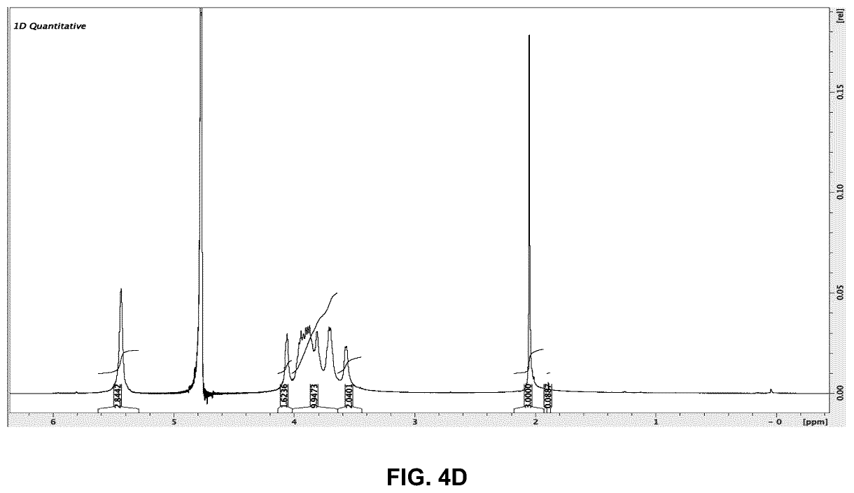

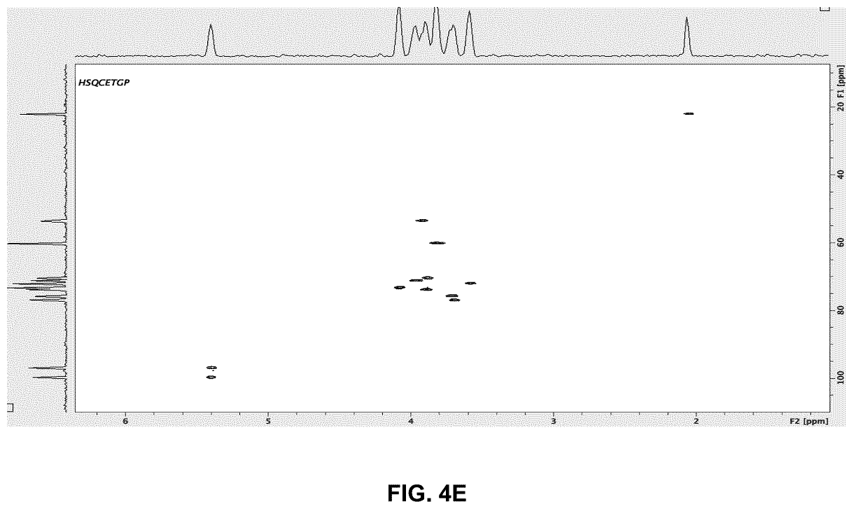

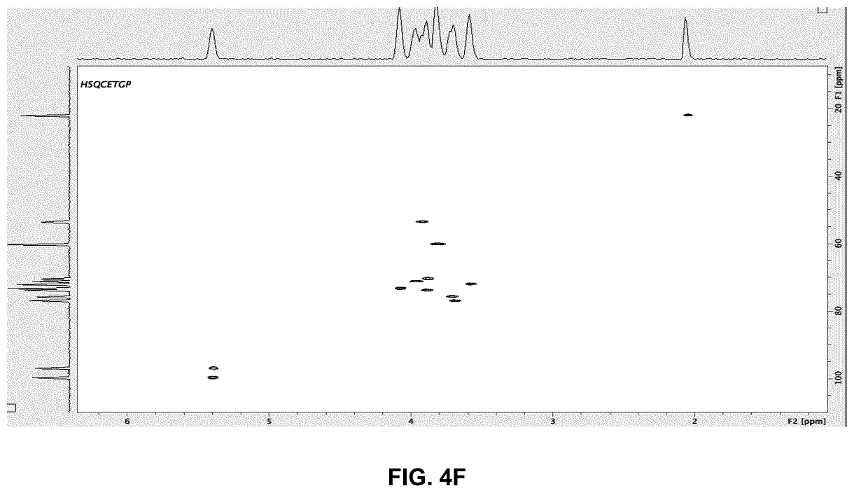

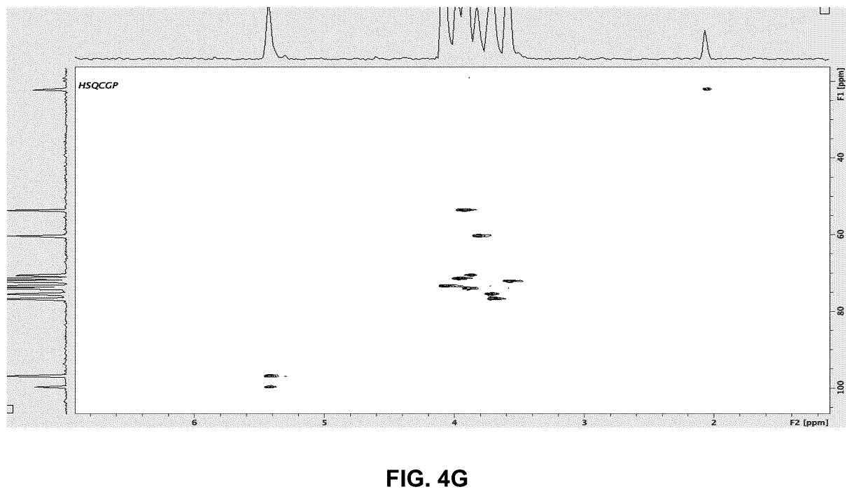

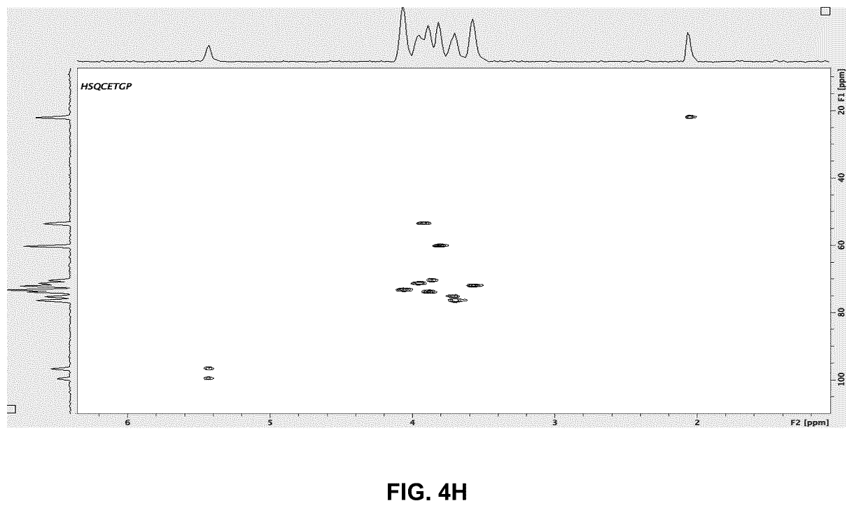

Referring now to FIG. 4A-H, NMR confirmation of structural identity of recombinant Testan glycosaminoglycans produced by CtTS, d64-CtTS, CIPTS and d65-CIPTS is illustrated. Recombinant Testan samples produced by E. coli expressing the full length CtTS enzyme (CtTS) or a d64-CtTS enzyme (shown as SEQ ID NO:5 lacking 64 amino acids on the N-terminal portion of full length CtTS shown as SEQ ID NO:1) (sometimes referenced herein as truncated d64-CtTS) were analyzed by quantitative ¹H and ²D HSQC NMR. Spectra demonstrate identical resonance patterns between CtTS (FIG. 4A and FIG. 4E) and, and d64-CtTS (FIG. 4B and FIG. 4F) samples and excellent agreement with the proposed repeating disaccharide of glucuronic acid (GlcUA) and n-acetylglucosamine (GlcNAc) ([-4-D-GlcUA-α1,4-D-GlcNAc-α1-]ₙ). Small (<4%) variation in aliphatic integrals arises from expected experimental error. Comparison with a reference in vitro synthesized Testan using full length CtTS (in vitro CtTS) (FIG. 4D and FIG. 4H) shows only minimal shifts, attributable to minor concentration or ionic differences rather than structural variation. These data also demonstrate that removal of the N-terminal 64 residues does not alter enzymatic function or product structure, confirming that this region is dispensable for glycosyltransferase activity.

Referring now to FIG. 4C and FIG. 4G, the corresponding recombinant glycosaminoglycan sample (d65-CIPTS enzyme variant) yields a ¹H NMR spectrum with integrals matching theoretical predictions and identical chemical shifts to the in vitro CtTS reference, confirming that the d65-CIPTS enzyme catalyzes formation of the same [-4-D-GlcUA-α1,4-D-GlcNAc-α1-]n polymer. These data establish structural equivalence between recombinant and in vitro polymers (FIG. 4D and FIG. 4H) and support the claimed compositions of matter comprising Testan polysaccharides.

### Example 1

The expression and activity of full-length CtTS and a truncated d64-CtTS testosteronan synthase enzyme in recombinant E. coli is provided according to one embodiment of the present invention.

### Methods

Genes encoding full-length CtTS and d64-CtTS were cloned into pET-28a(+) under T7 regulation. Host: BL21 (DE3) or T7 Express. Induction at OD₆₀₀=0.7 with 0.2 mM IPTG; temperature reduced to 20° C.; cultures grown 48 hours. The results indicate that both constructs produced high-MW Testan detectable in the culture supernatant. NMR Findings are illustrated in FIG. 4A for quantitative 1H Spectrum of CtTS-produced recombinant polymer and in FIG. 4B for quantitative 1H Spectrum of d64-CtTS-produced recombinant polymer. Full-length (CtTS) and d64-CtTS showed completely overlapping ¹H NMR spectra. 2D HSQC demonstrate coincident anomeric and aliphatic cross-peaks in FIG. 4E for CtTS produced recombinant polymer and FIG. 4F for d64-CtTS. Integral ratios matched theoretical expectations (3:10:2). This illustrates that the N-terminal 64 amino acids of CtTS are not required for Testan polymerization, enabling improved expression strategies using truncated enzymes.

### Example 2

Expression of d65-CIPTS (SEQ ID NO:7) Pseudomonas CIP10 Enzyme. To evaluate whether the d65-CIPTS homolog can produce Testan identical to CtTS-derived polymer. d65-CIPTS was codon-optimized for E. coli and cloned into pET-28a(+). Expression was performed as above in example 1. The results indicate that d65-CIPTS produced high-MW Testan (>800 kDa). The NMR Findings (FIG. 4A-H) indicate that d65-CIPTS-derived Testan is indistinguishable from CtTS Testan by both 1H NMR and HSQC. These results support d65-CIPTS as a functional Testan synthase, expanding the enzyme family capable of producing this polymer.

### Example 3

SAX Fractionation and MW/PDI Determination of recombinant Testan into defined MW populations is illustrated in Table 3. Crude Testan was applied to a strong-anion exchange (SAX) column and eluted using stepwise NaCl (0-1.0 M).

TABLE 3
Molecular Weight (MW) and Polydispersity Index (PDI) of Testan Fractions

| | MW 1 | MW 2 | MW 3 | Average | | | | Average |
|---|---|---|---|---|---|---|---|---|
| Sample | (kDa) | (kDa) | (kDa) | MW (kDa) | PDI 1 | PDI 2 | PDI 3 | PDI |
| Crude Unfractionated Testan | 791 | 834 | | 812.50 | 1.21 | 1.20 | | 1.21 |
| SAX Purified 0.5M NaCl | 495 | 452 | | 473.50 | 1.02 | 1.04 | | 1.03 |
| SAX Purified 1M NaCl | 832 | 970 | 931 | 911.00 | 1.02 | 1.02 | 1.02 | 1.02 |

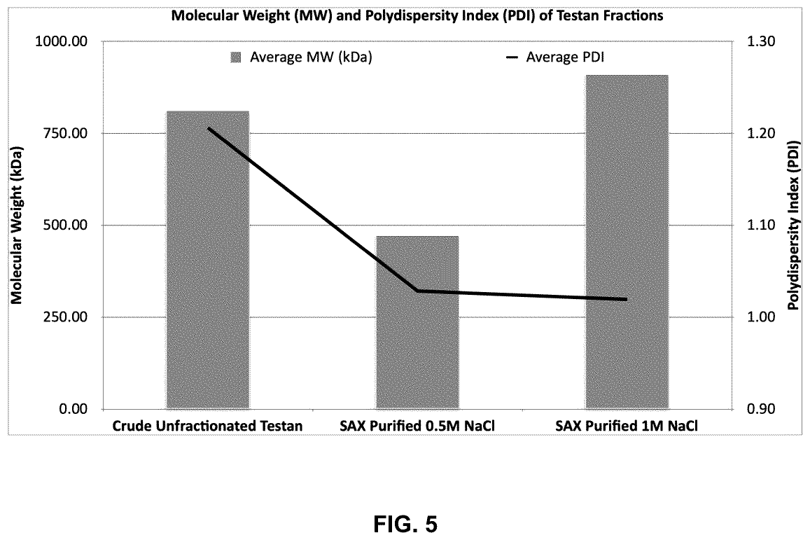

Referring now to FIG. 5, molecular weight and polydispersity analysis of recombinant Testan fractions is illustrated. Size-Exclusion Chromatography coupled with Multi-Angle Laser Light Scattering (SEC-MALLS) analysis of recombinant Testan samples before and after anion-exchange (SAX) purification. The Crude Unfractionated Testan sample exhibited an average molecular weight (MW) of ~812 kDa and a moderate polydispersity index (PDI) of 1.21, consistent with a heterogeneous mixture of polymer chain lengths produced directly from the fermentation supernatant. Fractionation by strong anion exchange (SAX) chromatography yielded two purified fractions distinguished by ionic strength:

The 0.5 M NaCl eluate displayed a lower average molecular weight (~474 kDa) and near-monomodal distribution (PDI≈1.03), corresponding to shorter chain-length polymers with minimal heterogeneity. The 1 M NaCl eluate retained a high molecular weight (~911 kDa) but showed an exceptionally narrow distribution (PDI≈1.03), indicating a homogeneous population of long-chain polymers. These results demonstrate that SAX fractionation effectively resolves Testan polysaccharides by size and charge, producing highly uniform preparations suitable for analytical and commercial applications. The low PDI values (<1.03) confirm consistent biosynthetic processing and precise control of polymer chain length in the recombinant system.

### Example 4

NMR Characterization of Recombinant Testan to confirm polymer identity and structural consistency across enzyme variants. Sammples were prepared in D₂O and analyzed by 1H NMR and HSQC at 600 MHz. Full-length CtTS (SEQ ID NO:1) Testan, d64-CtTS (SEQ ID NO:5) Testan, d65-CIPTS (SEQ ID NO:7) Testan, and reference Testan overlay perfectly. No additional peaks indicating heparosan, HA, or other contaminants. Anomeric cross-peaks at ~4.55/102.4 ppm (GlcA) and ~4.65/100.1 ppm (GlcNAc) are seen. Further there are no additional peaks indicating heparosan, HA, or other contaminants. Therefore, all recombinant enzymes produce the same Testan structure, validating the platform.

### Example 5

Rheological and Viscosity Properties of Recombinant Testan. To compare the rheological properties of recombinant Testan to heparosan and water using Cannon-Manning viscometers Two Cannon-Manning semi-micro viscometers were used: Type 150 (wide bore; working range 7-35 mm²/s) and Type 75 (narrow bore; working range 2-10 mm²/s). Polymers analyzed: Heparosan (169.7 kDa), at 10 mg/mL and 2.7 mg/mL and Recombinant Testan (813 kDa), at 2.7 mg/mL.

TABLE 4
Rheological Properties of Testan vs. Heparosan Polymers

| | MW | Concentration | Viscometer | Avg KV | |
|---|---|---|---|---|---|
| Polymer | (kDa) | (mg/ml) | Type | (mm²/s) | Notes |
| Heparosan | 170 | 10 | 150 | 21.1 | |
| Heparosan | 170 | 2.7 | 75 | 5.3 | |
| Testan | 813 | 2.7 | 150 | 2.7 | out or range for |
| | | | | | Type 150 (range 7-35) |
| Testan | 813 | 2.7 | 75 | 2.4 | |

TABLE. 4 illustrates Kinematic viscosity (KV) analysis of recombinant Testan (Mw≈813 kDa, crude 05/25 preparation) and reference heparosan (Mw≈170 kDa) in aqueous solution, measured by semi-micro Cannon-Manning viscometers (types 150 and 75) at room temperature. Viscosity was determined from replicate flow times according to the manufacturer's calibration equations (Cannon Instrument Co., Viscosity & Rheology Classroom and Semi-Micro Viscometer Manual).

For heparosan, the type 150 instrument at 10 mg/mL yielded an average KV≈21 mm²/s, while dilution to 2.7 mg/mL (type 75) reduced KV to ≈5.3 mm²/s, consistent with Newtonian scaling of viscosity with concentration. In contrast, Testan at the same 2.7 mg/ml concentration showed an apparent KV≈2.4 mm²/s using the type 75 viscometer-less than half the viscosity of heparosan despite a molecular weight nearly fivefold greater.

Measurements with the wider-bore type 150 viscometer returned an average KV≈2.7 mm²/s, below the reliable range (7-35 mm²/s) specified by the manufacturer, confirming extremely low flow resistance. For reference, pure water measured ≈0.86 mm²/s under identical conditions.

Interpretation: The crude recombinant Testan polymer is roughly six-times more viscous than water yet only ≈45% as viscous as the smaller heparosan polymer at equal concentration. This indicates that Testan adopts a compact, low-hydration hydrodynamic conformation, likely due to chain rigidity, acetyl shielding, or intramolecular hydrogen bonding. The observed behavior demonstrates non-ideal flow and reduced coil expansion relative to heparosan, distinguishing Testan as a structurally unique glycosaminoglycan with favorable solution and processing properties for formulation and chromatographic applications. Kinematic viscosity (KV) values were recorded for each flow time.

### Example 6

Preparation of Sulfated Testan (sTestan) derivatives were generated and their biochemical activity was assessed. Testan was sulfated using an aqueous sulfation method (e.g., sulfur trioxide-pyridine or chlorosulfonic acid adaptation) optimized to preserve polymer integrity. The results of migration on agarose gels illustrate that Sulfated Testan migrated more rapidly on agarose gels (FIG. 5), consistent with increased negative charge density. sTestan is stable, water-soluble, and retains polymer integrity after sulfation.

### Example 7

Stains-All Agarose Gel Analysis of Testan and sTestan. To characterize unsulfated and sulfated Testan on a Stains-All agarose gel and define their electrophoretic mobility relative to HA standards. A 1-2% agarose gel was cast and stained with Stains-All. Agarose gel electrophoresis showing Testan samples and sulfated Testan. Lane 1: HA HiLadder; Lane 3: unsulfated Testan; Lane 5: sulfated Testan; Lane 7: HA LoLadder. Differences in mobility reflect changes in charge and hydrodynamic properties. Unsulfated Testan migrates as a high-MW band between HA standards. Sulfated Testan migrates more rapidly, consistent with increased charge density. No additional contaminating bands observed. Stains-All electrophoresis further confirms polymer integrity and sulfation efficiency

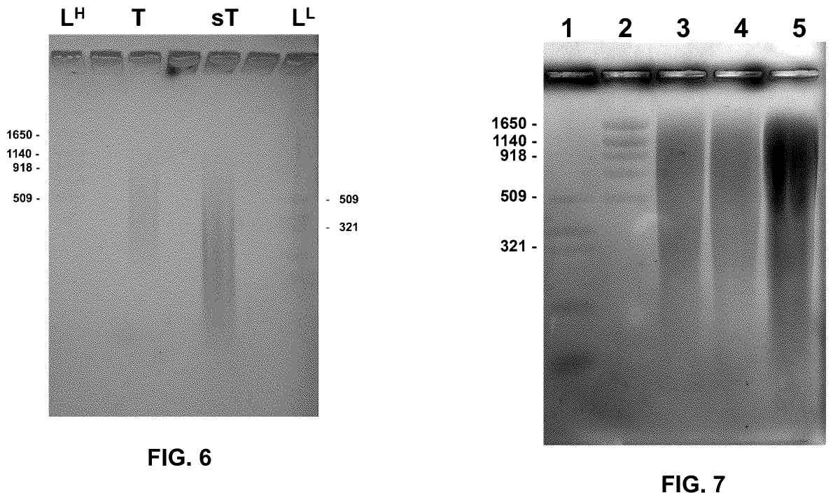

Referring now to FIG. 6, an agarose gel electrophoresis (Stains-All stain) comparing unsulfated Testan (T) and sulfated Testan (sT) samples to HA molecular-weight standards. Lane 1: HA HiLadder (Hyalose). Lane 3: unsulfated Testan. Lane 5: sulfated Testan produced by aqueous sulfation. Lane 7: HA LoLadder (Hyalose). The sulfated Testan stains a purple rather than blue color, and migrates more rapidly than the unsulfated polymer, consistent with increased negative charge density and altered hydrodynamic properties following sulfation of the Testan backbone.

Referring now to FIG. 7, an agarose gel electrophoresis (0.8% agarose, Stains-All stain) comparing recombinant Testan samples to commercial HA standards. Lane 1: HA LoLadder (Hyalose). Lane 2: HA HiLadder (Hyalose). Lane 3: recombinant Testan produced by full-length CtTS. Lane 4: recombinant Testan produced by the d64-CtTS truncation variant. Lane 5: recombinant Testan produced by the d65-CIPTS homolog. All recombinant Testan samples migrate as high-molecular-weight, Stains-All-positive polysaccharides distinct from the HA standards, demonstrating successful polymer production by each synthase.

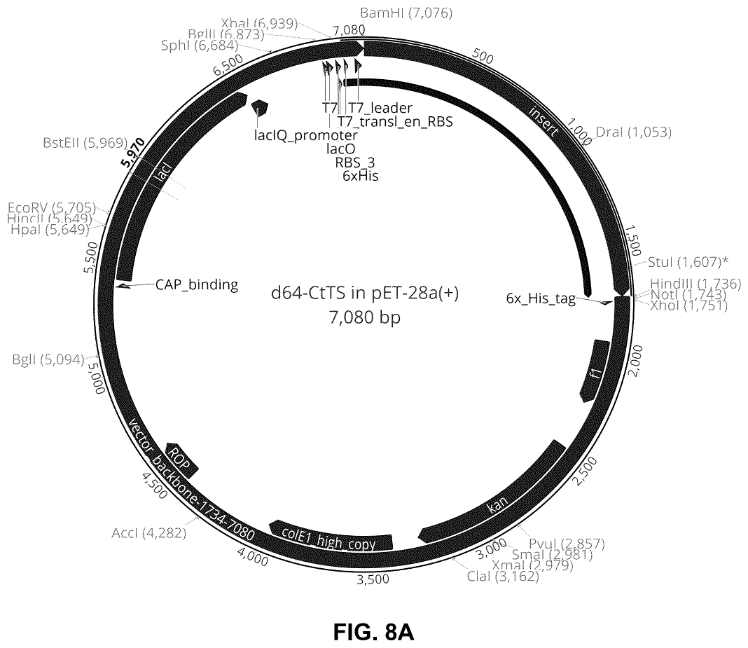

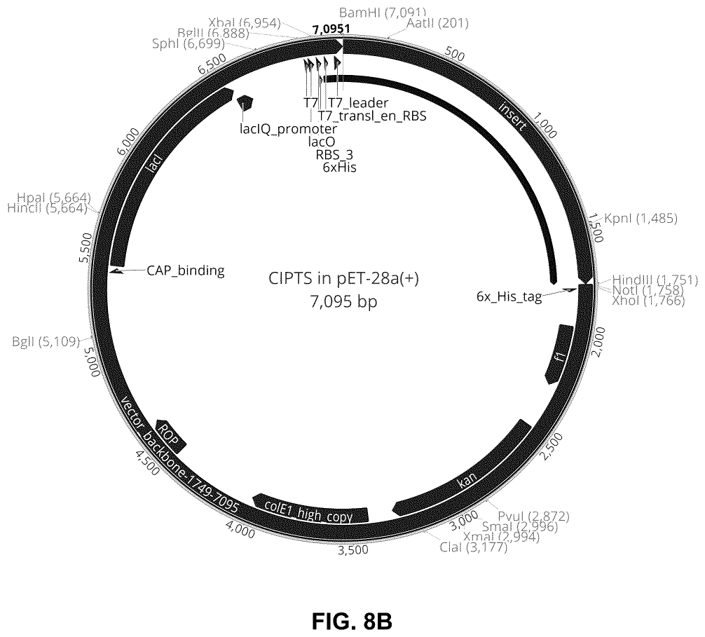

Referring now to FIG. 8A-B, illustrates maps of pET-28a(+) vectors expressing d64-CtTS (FIG. 8A) and d65-CIPTS (FIG. 8B).

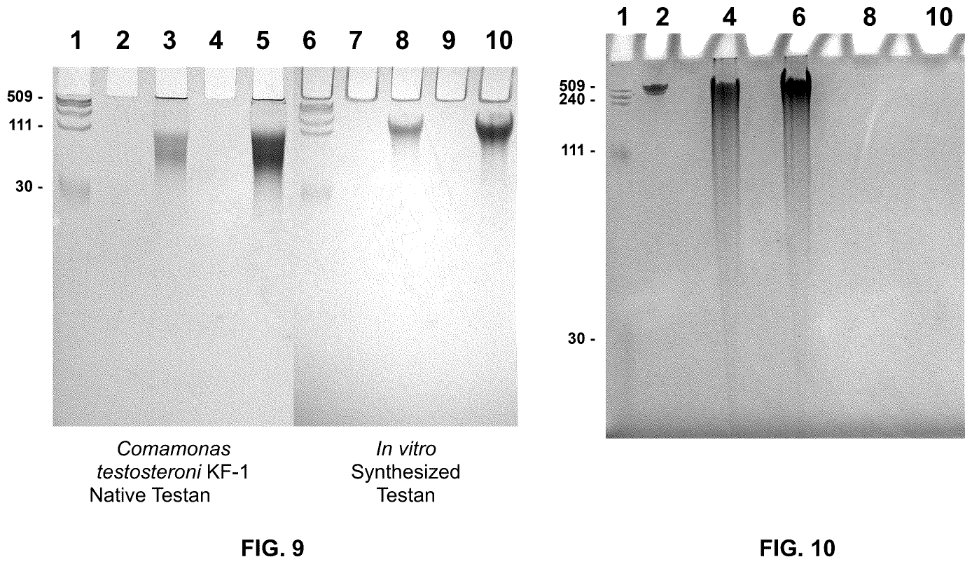

Referring now to FIG. 9, TBE-PAGE (6%) gel analysis of Testan samples stained with Alcian Blue is illustrated. Lane 1: HA LoLadder (Hyalose). Lane 3: Testan extracted from Comamonas testosteroni KF-1 (1 μL loaded; apparent molecular weight ~60 kDa). Lane 5: Testan extracted from C. testosteroni KF-1 (5 μL loaded). Lane 6: HA LoLadder. Lane 8: in vitro-synthesized Testan produced by purified CtTS (0.1 μL loaded; apparent molecular weight ~100 kDa). Lane 10: in vitro-synthesized Testan produced by purified CtTS (1 μL loaded). For comparison, recombinant Testan produced in the engineered microbial system described herein exhibits a molecular weight approximately ten-fold higher than the native polymer (~800-900 kDa by MALLS), consistent with the distinct high-molecular-weight Testan fractions obtained by recombinant expression. All Testan samples migrate as Alcian Blue-reactive polysaccharides distinct from HA standards.

Referring now to FIG. 10, Alcian Blue-stained 6% TBE-PAGE gel analysis comparing Testan production by multiple heterologous Testan synthase homologs expressed in E. coli is illustrated. Lane 1 contains the Hyalose HA LoLadder, and Lane 2 contains the HA HiLadder. Lane 4 shows the SEQ ID NO:5 (d64-CtTS) recombinant product, and Lane 6 shows the SEQ ID NO:7 (d65-CIPTS) recombinant product, both of which migrate as high-molecular-weight Alcian Blue-reactive polysaccharides consistent with Testan. Lanes 8 and 10 contain extracts from E. coli expressing SEQ ID NO:3 (SmTS) and SEQ ID NO:4 (ApTS), respectively, neither of which show Alcian Blue-positive high-molecular-weight bands. These results demonstrate that Testan is produced only by CtTS-derived and CIPTS-derived synthases and not by SmTS or ApTS under the same expression conditions.

The preceding examples can be repeated with similar success by substituting the generically or specifically described reactants and/or operating conditions of an embodiment of the present invention for those used in the preceding examples.

Table 6. Candidate Residue Substitutions To Enable Testan Synthesis by SEQ ID NO: 3. Table 5 lists amino-acid positions in SEQ ID NO:3 (a non-functional Testan synthase homolog from Candidatus Symbiobacter mobilis) that differ from the corresponding residues in the functional consensus Testan synthase sequence (SEQ ID NO:8). For each position, the residue present in SEQ ID NO:3 and the aligned residue in SEQ ID NO:8 are shown (See FIG. 12). One or more substitutions at these positions may be required to restore or enable Testan polymerase activity. Positions include residues impacting donor-sugar binding, acceptor-site positioning, catalytic loop geometry, metal-coordination motifs, and overall fold stability. The listed differences represent rational engineering targets for generating functional variants of SEQ ID NO: 3.

TABLE 6

| SEQ ID NO: 3 position | SEQ ID NO: 3 AA | SEQ ID NO: 8 AA |
|---|---|---|
| 83 | V | I |
| 87 | M | I |
| 92 | D | E |
| 100 | S | T |
| 123 | S | D |
| 135 | M | F |
| 140 | I | L |
| 155 | Y | I/L |
| 178 | R | K |
| 179 | H | E |
| 181 | H | N |
| 182 | G | Y |
| 192 | V | L |
| 207 | K | R |
| 210 | L | V |
| 212 | G | S |
| 213 | S | A |
| 220 | A | P |
| 236 | M | L |
| 238 | V | I |
| 239 | R | K |
| 241 | A | S |
| 245 | A | H |
| 254 | A | S |
| 255 | L | I |
| 256 | N\| | K |
| 259 | K | E |
| 265 | Y | I |
| 272 | G | N/D |
| 273 | A | E |
| 275 | R | T |
| 288 | D | H |
| 295 | T | S |
| 296 | R | K |
| 306 | L | F |
| 314 | L | V |
| 330 | N | E |
| 332 | F | Y |
| 333 | Q | S |
| 345 | Q | K |
| 357 | V | I |
| 362 | H | N |
| 388 | A | S |
| 392 | E | D |
| 423 | R | K |
| 446 | H | N |
| 465 | E | L |
| 467 | P | T |
| 468 | A | P |
| 470 | T | E |
| 481 | V | I |
| 494 | I | V |
| 500 | H | N |
| 507 | F | H |
| 508 | R | N |
| 509 | K | R |
| 510 | M | L |
| 512 | Y | H |
| 518 | F | Y |
| 524 | D | N |
| 528 | S | P |
| 529 | R | K |
| 531 | Q | K |
| 539 | E | N |
| 545 | G | A |
| 551 | L | I |
| 555 | I | V |
| 557 | T | L |
| 563 | S | A |
| 569 | C | Y |
| 588 | T | A |
| 604 | T | A |
| 614 | E | D/N |
| 615 | R | K |

TABLE 7
SEQUENCES for SEQ ID NO: 1-10 and 14-16

| SEQ | |
|---|---|
| ID | |
| NO: | Sequence |
| 1 | MSGMFKVAND FFSNGNFEKA IERYEEIIFK YPGLTEFASG NLALARRKLG ERQENKSKSL 60 |
| | VNASKISESI FVGIAAIPER AKALEKTIES LLPQVEKIGV YLNGWKEVPD YLKNEKILVE 120 |
| | GFGKEDLGDV GKFFWVDQHD GIYFSCDDDL IYPKDYVDRT VEKLKEKNYK AAIGWHGSLL 180 |
| | RDNFSTYYDK NSRRVFVFSA HRPWDTPVHI LGTGCSAFHT KFLKIKKSDF LHPNMADIFF 240 |
| | SIKGQEQKIP FIVLAHEKDE ITEFVGAKES SIYSHSQANV ESKKNTHDLQ NGFVMKNMPW 300 |
| | VMNDVESLSV LIVGRFENYS KGGIYKSCHL IKEHLSALGH DVDIHDTQNP FAKALEKKYD 360 |
| | LCWIYPGDPE RPDFSSVEDK IYELKSRGIP VIVNLSYLYS EDRTIWIRNK IRDLNAKGTT 420 |
| | PVLGAVFTET AANDPLLKDV RDYICVVPKT ILPTPCERYY EFGEREGICL GDATKLGNAK 480 |
| | VIGGNVNNWI DAIHNRLPHV NLYAYKQYQG NNPHPKIKYA PHMKENFGDW LAQRRIFICL 540 |
| | NVHLTFEMVA CEAQSYGTPV IYRHMPHSLS EYISATGFAT RSPDEMAEMV AWLYNNNKAW 600 |
| | NKMSQASLNN GKANNVNLLD SSLEGYLRLA ILRIKKMMVK 640 |
| 2 | atgagtggaa tgtttaaggt tgccaatgat tttttctcta acggtaattt tgaaaaagca 60 |
| | attgaacgtt atgaggaaat aatttttaag tatccaggct tgacagagtt tgcgtcaggg 120 |
| | aatttagcgc tggcaagaag aaaactagga gagcgccaag aaaataaatc taaatcattg 180 |
| | gtgaatgctt ccaagatatc tgagagtatt tttgtaggta tagcagctat accagaacgc 240 |
| | gcgaaagctt tagagaaaac tattgagtct ttattgcctc aggtagaaaa aataggggtt 300 |
| | tacttaaacg gttggaagga agtcccagat tacttgaaga atgagaaaat ccttgtagag 360 |
| | ggattcggca aagaagacct gggagacgtg ggtaagtttt tttgggtgga tcagcatgat 420 |
| | ggaatctatt tctcatgtga tgacgactta atctatccaa aggactatgt tgatagaaca 480 |
| | gttgaaaagt taaaagagaa aaattacaaa gctgcaattg gctggcatgg ttctctattg 540 |
| | agagataatt ttagtactta ttacgataag aattctcgac gtgtttttgt tttttctgca 600 |
| | catcgtccat gggatacgcc tgtacatatt ctaggtacag gatgctcagc gttccatact 660 |
| | aagttcttaa agataaagaa gtctgatttt ctgcatccaa atatggcgga tatatttttc 720 |
| | tccattaaag ggcaggaaca aaaaatacct tttattgtct tggcgcatga aaaagatgaa 780 |
| | ataacagagt ttgttggggc taaggaaagc tctatttact cgcattctca ggcgaatgtg 840 |
| | gaatcgaaaa aaaataccca tgatttgcaa aatggttttg taatgaaaaa catgccatgg 900 |
| | gtcatgaatg atgtcgagtc tctatctgtt ttgatagttg gtagatttga aaattatagt 960 |
| | aaaggtggga tctataagtc atgccatctt attaaagagc atctgtcggc actgggtcat 1020 |
| | gatgttgata ttcatgatac tcaaaatcca ttcgccaagg cgttagagaa aaaatatgat 1080 |
| | ctgtgttgga tatacccagg tgatcctgag cgaccagatt tttctagtgt tgaagataaa 1140 |
| | atttatgaac tgaagtcaag gggtattccg gtaatcgtta atctctctta tttgtattct 1200 |
| | gaagatcgaa ccatatggat togaaataaa atacgagatt tgaatgcgaa aggaactact 1260 |
| | cctgtacttg gagctgtgtt tacagaaact gccgcaaatg atccgctgct aaaagatgtg 1320 |
| | agagactata tttgcgtcgt toctaagacg atactaccta caccttgtga aaggtattat 1380 |
| | gagtttggtg agcgagaggg tatatgtttg ggggatgcaa cgaaactcgg gaatgctaaa 1440 |
| | gttataggcg gtaatgttaa taattggatt gatgcaattc acaatagact tcctcatgtc 1500 |
| | aatctttatg catacaagca gtatcaaggt aataacccac atccaaagat taaatatgct 1560 |
| | ccacatatga aagagaattt tggtgattgg ttggcgcaac gtaggatatt tatctgtttg 1620 |
| | aacgttcatt taacatttga gatggtggcg tgcgaggcgc aaagttacgg tacacctgtg 1680 |
| | atttatagac atatgccgca ctccttgagt gagtatatct ctgctacggg gtttgcaact 1740 |
| | cggtcacctg atgaaatggc tgaaatggtg gcatggttat ataataataa caaggcgtgg 1800 |
| | aataagatga gtcaagcttc tctaaataat gggaaggcta ataatgttaa tttattagac 1860 |
| | tcctctcttg aggggtattt gagattggct attctcagaa ttaaaaagat gatggtgaaa 1920 |
| | tga |
| 3 | MQSQNNHPPA QPGTQAQVRH AYALFKQGRY PEAMEAYERI LASHPEMERR LRVNIHIAKK 60 |
| | RIQNGIRTRQ TLARDDKELS NAVFVGMAAI PDRVAALEAS ICSLLPQAER IGVYLNGWEQ 120 |
| | IPSFLQHEKI IVAGMGEPDI GDVGKFHWVD DHDGYYFTCD DDLIYPPDYI ARTLAKLRHY 180 |
| | HGNAAVGWHG SVLMEPFARY YDTNSRKVFL FGSHRPHDTA VHILGTGCCA FHTQVMQVRK 240 |
| | ADFTAPNMAD IFFALNGQKQ KIPFYVIEHE KGAIREAPGT KESSIYADSH QNKSTRKNTH 300 |
| | ELQNQLVRSN APWLLHEFQP LSILIIGRFN SFQKGGIYKS CHLIQEHLSN LGHHVSVLDT 360 |
| | QHALVLPESA HFDLCWIYPG DPERPDFATV VEKIRSLQKR GIPVLVNLSY LYQEKRTTWI 420 |
| | RDRILECNAG QSTPVLCAVF TESAAHDPLL ADVRDYVCVV PKTIEPPACT KVRGFGEREG 480 |
| | VCLGDATKLS NPKIIGGPVH AWIDAIFRKM PYVNLYAFKQ YQGDNPHSRV QYVPHMKEEF 540 |
| | GDWLGQRRLF LCLNIHTTFE MVSCEAQYCG TPVLYRHMPH SLSEYISTTG MAVRNPEEMA 600 |
| | EMVTWLYNDP AAWERCSRSS LRNAESKHVD VLDASLEGYL RLAVQRARRL TKPATSTSDQ 660 |
| | RC 662 |
| 4 | MSLKEANSLV RQDKLLEAKK IYNKIYKSSS PTIREQIEFN LKLINKKLLN NGNHKSFSVH 60 |
| | LSFEEPTFVG IASIPSREYA LLETVKSLVK QVDKIGIFLD GYSEIPDFLK DNKKILIKRS 120 |
| | QDFDRKVGDA GKFFWVDEHK GFYFTCDDDL IYPNDYVARI KQKIISKREP VVVGWHGSLI 180 |
| | LCPFKNYYNK NSRRVFTFGS ARPEDTPVHI LGTGCVGFHT SSFNVNFCDF PTPNMADVYF 240 |
| | AKLGQEQKVP FIVIKHEKEE IQEVPDTREV SIYQHSHSNK DTEHNTKTTQ NEVVSSIDWN 300 |
| | VNYLKNSLDI LIVGRFLINE KGGIFKSSRL LVEGLTQLGH RVSTCCLSQI DEFDFSSKKF 360 |
| | DFSIIYAPDP ERPDFSNCID KVQLMIRLGV VCAVNFSFNL NENRTRWIER KLTELNSPFS 420 |
| | YPRCFFASFS NSTSALFSED ISSMIVPFPK TISLNLKGKE RLRYSEREGI FLGDLAKLLD 480 |
| | EKLTHGNLLP WIEELSKRLP HVNLYAIKHY HTDLDYPKNI KVIPYTKNIE NVLGNYRLCI 540 |
| | NLTPGATFEM VPLEALLSGT PVLHRRMPQS LSEYLSPVSI EINSPKELGE VCQRIYENEN 600 |
| | IWNRLSKAGS GSYEFFRIEN ITAALDLSIR KCLNRSGN 638 |
| 5 | SIFVGIAAIP ERAKALEKTI ESLLPQVEKI GVYLNGWKEV PDYLKNEKIL VEGFGKEDLG 60 |
| | DVGKFFWVDQ HDGIYFSCDD DLIYPKDYVD RTVEKLKEKN YKAAIGWHGS LLRDNFSTYY 120 |
| | DKNSRRVFVF SAHRPWDTPV HILGTGCSAF HTKFLKIKKS DFLHPNMADI FFSIKGQEQK 180 |
| | IPFIVLAHEK DEITEFVGAK ESSIYSHSQA NVESKKNTHD LONGFVMKNM PWVMNDVESL 240 |
| | SVLIVGRFEN YSKGGIYKSC HLIKEHLSAL GHDVDIHDTQ NPFAKALEKK YDLCWIYPGD 300 |
| | PERPDFSSVE DKIYELKSRG IPVIVNLSYL YSEDRTIWIR NKIRDLNAKG TTPVLGAVFT 360 |
| | ETAANDPLLK DVRDYICVVP KTILPTPCER YYEFGEREGI CLGDATKLGN AKVIGGNVNN 420 |
| | WIDAIHNRLP HVNLYAYKQY QGNNPHPKIK YAPHMKENFG DWLAQRRIFI CLNVHLTFEM 480 |
| | VACEAQSYGT PVIYRHMPHS LSEYISATGF ATRSPDEMAE MVAWLYNNNK AWNKMSQASL540 |
| | NNGKANNVNL LDSSLEGYLR LAILRIKKMM VK 572 |
| 6 | MDKLNTPSNL ATANQLFRAK KYSEAALFYK KSIQESPELA EAIRINLHLC AKRAPAGEIE 60 |
| | QITFQNNNED NIFVGIAAIP ERETALKQTI ASLINQAPRI GVYLNGWKRI PDFLRHEKIE 120 |
| | IAGFHDADLG DVGKFHWVDN HDGLYFTCDD DLVYPPDYIK RTAAKLKEHN YRAAVGWHGS 180 |
| | LIKEPFVNYY DGASRRVFVF SAHRPVDTPV HILGTGCCAF HTRELSIKKS DFVHPNMADI 240 |
| | FFSIKGQEQN IPFIVIKHEK NEITEVEGSK ESSIFAHSHT DQASKKNTKE IQNTFVSNNL 300 |
| | PWVLKSFQPF SILIIGRFES YSKGGIYKSC HLIKKHLTEL GHNVSILDTG NEMTAEHVSG 360 |
| | HDICWIYPGD PERPDFSTVD DKIHLLRKHG VPVLLNLSYL YESHRSQWIV EKLKEYNSQA 420 |
| | GTPVLAAVFT ESAANDPLLN SVRDYVCVVP KTLLPTPYEM VPSFDQREGI CLGDATKLGN 480 |
| | PIVIGGSINP WIDAIHNRLP HVNIYAYKQY QGNNPHPKVK YVSHMKENFG EWLAQRRLFI 540 |
| | CANVHLTFEM VACEAQQYGT PTLYRHMPHS LSEYISATGM AVRTPGEMGE MAAWLYNSRA 600 |
| | TWDKLSASSI HNANSKHVDL LDASLEGYIR LAIYRASLLK SKFLTKK 647 |
| 7 | SIFVGIAAIP ERETALKQTI ASLINQAPRI GVYLNGWKRI PDFLRHEKIE IAGFHDADLG 60 |
| | DVGKFHWVDN HDGLYFTCDD DLVYPPDYIK RTAAKLKEHN YRAAVGWHGS LIKEPFVNYY 120 |
| | DGASRRVFVF SAHRPVDTPV HILGTGCCAF HTRELSIKKS DFVHPNMADI FFSIKGQEQN 180 |
| | IPFIVIKHEK NEITEVEGSK ESSIFAHSHT DQASKKNTKE IQNTFVSNNL PWVLKSFQPF 240 |
| | SILIIGRFES YSKGGIYKSC HLIKKHLTEL GHNVSILDTG NEMTAEHVSG HDICWIYPGD 300 |
| | PERPDFSTVD DKIHLLRKHG VPVLLNLSYL YESHRSQWIV EKLKEYNSQA GTPVLAAVFT 360 |
| | ESAANDPLLN SVRDYVCVVP KTLLPTPYEM VPSFDQREGI CLGDATKLGN PIVIGGSINP 420 |
| | WIDAIHNRLP HVNIYAYKQY QGNNPHPKVK YVSHMKENFG EWLAQRRLFI CANVHLTFEM 480 |
| | VACEAQQYGT PTLYRHMPHS LSEYISATGM AVRTPGEMGE MAAWLYNSRA TWDKLSASSI 540 |
| | HNANSKHVDL LDASLEGYIR LAIYRASLLK SK 572 |
| 8 | MXXXXXXXXX XXANXXFXXX XXXXAXXXYX XXIXXXPXLX EXXXXNLXLX XXXXXXXXEX 60 |
| | XXXXJXXXXX XXXXIFVGIA AIPERXXALX XTIXSLJXQX XXIGVYLNGW KXXPDXLXXE 120 |
| | KIXXXGFXXX DLGDVGKFXW VDXHDGJYFX CDDDLXYPXD YXXRTXXKLK EXNYXAAXGW 180 |
| | HGSLJXXXFX XYYDXXSRRV FVFSAHRPXD TPVHILGTGC XAFHTXXLXI KKSDFXHPNM 240 |
| | ADIFFSIKGQ EQXIPFIVJX HEKBEITEXX GXKESSIXXH SXXBXXSKKN TXXJQNXFVX 300 |
| | XNXPWVXXXX ZXXSXLIXGR FEXYSKGGIY KSCHLIKXHL XXLGHBVXIX DTXNXXXXXX 360 |
| | XXXXDJCWIY PGDPERPDFS XVXDKIXXLX XXGXPVJXNL SYLYXXXRXX WIXXKJXXXN 420 |
| | XXXXTPVLXA VFTEXAANDP LLXXVRDYXC VVPKTJLPTP XEXXXXFXZR EGICLGDATK 480 |
| | LGNXXVIGGX XNXWIDAIHN RLPHVNJYAY KQYQGNNPHP KXKYXXHMKE NFGXWLAQRR 540 |
| | JFICXNVHLT FEMVACEAQX YGTPXJYRHM PHSLSEYISA TGXAXRXPXE MXEMXAWLYN 600 |
| | XXXXWBKXSX XSJXNXXXXX VBLLDXSLEG YJRLAIXRXX XXXXKFLTKK 650 |
| 9 | TCCAAAATTTCAGAGTCAATTTTCGTAGGTATTGCCGCCATACCGGAGCGCGCCAAAGCA |
| | CTGGAGAAAACAATCGAGTCGCTGTTACCGCAGGTTGAAAAGATCGGTGTGTACTTGAAC |
| | GGGTGGAAAGAGGTACCAGATTATCTTAAGAATGAGAAAATTCTCGTCGAAGGATTTGGA |
| | AAAGAGGACCTGGGCGACGTAGGCAAATTCTTCTGGGTTGATCAGCATGACGGGATTTAT |
| | TTTAGCTGCGACGATGATTTAATCTATCCGAAAGATTATGTGGATCGTACCGTTGAGAAA |
| | CTTAAAGAGAAGAACTACAAAGCGGCTATCGGCTGGCATGGCTCGTTGTTACGTGATAAT |
| | TTCTCAACGTATTATGATAAGAATAGTCGTCGCGTATTTGTATTTAGTGCTCATCGTCCA |
| | TGGGATACTCCAGTGCATATCTTAGGAACAGGGTGTTCTGCCTTTCATACAAAATTCTTG |
| | AAAATCAAGAAGTCTGACTTCCTGCACCCCAATATGGCGGACATTTTCTTTTCTATTAAG |
| | GGGCAAGAGCAGAAAATTCCTTTTATCGTTTTGGCACATGAAAAGGATGAAATTACGGAA |
| | TTTGTTGGCGCAAAAGAGTCATCAATCTATAGTCACAGTCAAGCCAATGTGGAGAGTAAG |
| | AAGAACACTCATGATCTCCAGAACGGCTTCGTGATGAAGAATATGCCTTGGGTCATGAAT |
| | GACGTTGAATCTCTGTCAGTCTTGATAGTCGGGCGCTTTGAAAACTATTCAAAAGGTGGG |
| | ATATACAAGTCGTGTCACCTCATCAAGGAACATCTTTCCGCTCTGGGCCACGATGTGGAT |
| | ATTCATGATACGCAGAACCCTTTCGCAAAAGCGCTTGAGAAGAAGTATGATCTTTGCTGG |
| | ATATACCCCGGAGATCCAGAACGACCAGATTTTAGTTCGGTGGAAGATAAAATCTACGAG |
| | TTAAAGAGTCGAGGAATCCCAGTTATAGTGAATCTGTCATACCTGTATTCAGAGGATCGG |
| | ACAATCTGGATTCGTAACAAAATTCGCGATTTAAACGCCAAAGGTACCACCCCCGTTCTG |
| | GGCGCGGTGTTTACAGAAACGGCTGCAAACGATCCGCTTCTTAAAGACGTTCGTGACTAT |
| | ATTTGTGTCGTTCCAAAGACAATTCTCCCGACTCCTTGTGAACGCTACTACGAGTTCGGC |
| | GAGAGAGAGGGTATTTGCTTGGGAGATGCTACCAAATTAGGTAACGCAAAGGTCATTGGC |
| | GGAAATGTGAATAACTGGATTGATGCCATCCACAATCGCTTACCTCATGTCAATCTGTAC |
| | GCCTATAAACAGTATCAAGGTAACAATCCTCACCCTAAAATTAAGTACGCCCCGCATATG |
| | AAGGAAAACTTCGGCGACTGGTTGGCGCAACGTCGAATCTTTATCTGTCTCAATGTGCAC |
| | TTAACCTTTGAGATGGTTGCGTGTGAGGCGCAATCGTACGGAACACCAGTGATTTATCGT |
| | CATATGCCACACTCACTTTCCGAATATATATCCGCCACGGGTTTCGCGACTAGATCCCCC |
| | GATGAAATGGCTGAAATGGTAGCGTGGCTGTATAACAACAATAAGGCCTGGAACAAAATG |
| | TCCCAAGCGAGTCTCAACAATGGGAAAGCCAACAATGTCAATCTGCTGGACAGCTCTTTG |
| | GAAGGGTATCTTCGTCTCGCCATATTGCGTATTAAGAAGATGATGGTCAAATGAAAGCTT |
| | GCGGCCGCACTCGAGCACCACCACCACCACCACTGAGATCCGGCTGCTAACAAAGCCCGA |
| | AAGGAAGCTGAGTTGGCTGCTGCCACCGCTGAGCAATAACTAGCATAACCCCTTGGGGCC |
| | TCTAAACGGGTCTTGAGGGGTTTTTTGCTGAAAGGAGGAACTATATCCGGATTGGCGAAT |
| | GGGACGCGCCCTGTAGCGGCGCATTAAGCGCGGCGGGTGTGGTGGTTACGCGCAGCGTGA |
| | CCGCTACACTTGCCAGCGCCCTAGCGCCCGCTCCTTTCGCTTTCTTCCCTTCCTTTCTCG |
| | CCACGTTCGCCGGCTTTCCCCGTCAAGCTCTAAATCGGGGGCTCCCTTTAGGGTTCCGAT |
| | TTAGTGCTTTACGGCACCTCGACCCCAAAAAACTTGATTAGGGTGATGGTTCACGTAGTG |
| | GGCCATCGCCCTGATAGACGGTTTTTCGCCCTTTGACGTTGGAGTCCACGTTCTTTAATA |
| | GTGGACTCTTGTTCCAAACTGGAACAACACTCAACCCTATCTCGGTCTATTCTTTTGATT |
| | TATAAGGGATTTTGCCGATTTCGGCCTATTGGTTAAAAAATGAGCTGATTTAACAAAAAT |
| | TTAACGCGAATTTTAACAAAATATTAACGCTTACAATTTAGGTGGCACTTTTCGGGGAAA |
| | TGTGCGCGGAACCCCTATTTGTTTATTTTTCTAAATACATTCAAATATGTATCCGCTCAT |
| | GAATTAATTCTTAGAAAAACTCATCGAGCATCAAATGAAACTGCAATTTATTCATATCAG |
| | GATTATCAATACCATATTTTTGAAAAAGCCGTTTCTGTAATGAAGGAGAAAACTCACCGA |
| | GGCAGTTCCATAGGATGGCAAGATCCTGGTATCGGTCTGCGATTCCGACTCGTCCAACAT |
| | CAATACAACCTATTAATTTCCCCTCGTCAAAAATAAGGTTATCAAGTGAGAAATCACCAT |
| | GAGTGACGACTGAATCCGGTGAGAATGGCAAAAGTTTATGCATTTCTTTCCAGACTTGTT |
| | CAACAGGCCAGCCATTACGCTCGTCATCAAAATCACTCGCATCAACCAAACCGTTATTCA |
| | TTCGTGATTGCGCCTGAGCGAGACGAAATACGCGATCGCTGTTAAAAGGACAATTACAAA |
| | CAGGAATCGAATGCAACCGGCGCAGGAACACTGCCAGCGCATCAACAATATTTTCACCTG |
| | AATCAGGATATTCTTCTAATACCTGGAATGCTGTTTTCCCGGGGATCGCAGTGGTGAGTA |
| | ACCATGCATCATCAGGAGTACGGATAAAATGCTTGATGGTCGGAAGAGGCATAAATTCCG |
| | TCAGCCAGTTTAGTCTGACCATCTCATCTGTAACATCATTGGCAACGCTACCTTTGCCAT |
| | GTTTCAGAAACAACTCTGGCGCATCGGGCTTCCCATACAATCGATAGATTGTCGCACCTG |
| | ATTGCCCGACATTATCGCGAGCCCATTTATACCCATATAAATCAGCATCCATGTTGGAAT |
| | TTAATCGCGGCCTAGAGCAAGACGTTTCCCGTTGAATATGGCTCATAACACCCCTTGTAT |
| | TACTGTTTATGTAAGCAGACAGTTTTATTGTTCATGACCAAAATCCCTTAACGTGAGTTT |
| | TCGTTCCACTGAGCGTCAGACCCCGTAGAAAAGATCAAAGGATCTTCTTGAGATCCTTTT |
| | TTTCTGCGCGTAATCTGCTGCTTGCAAACAAAAAAACCACCGCTACCAGCGGTGGTTTGT |
| | TTGCCGGATCAAGAGCTACCAACTCTTTTTCCGAAGGTAACTGGCTTCAGCAGAGCGCAG |
| | ATACCAAATACTGTCCTTCTAGTGTAGCCGTAGTTAGGCCACCACTTCAAGAACTCTGTA |
| | GCACCGCCTACATACCTCGCTCTGCTAATCCTGTTACCAGTGGCTGCTGCCAGTGGCGAT |
| | AAGTCGTGTCTTACCGGGTTGGACTCAAGACGATAGTTACCGGATAAGGCGCAGCGGTCG |
| | GGCTGAACGGGGGGTTCGTGCACACAGCCCAGCTTGGAGCGAACGACCTACACCGAACTG |
| | AGATACCTACAGCGTGAGCTATGAGAAAGCGCCACGCTTCCCGAAGGGAGAAAGGCGGAC |
| | AGGTATCCGGTAAGCGGCAGGGTCGGAACAGGAGAGCGCACGAGGGAGCTTCCAGGGGGA |
| | AACGCCTGGTATCTTTATAGTCCTGTCGGGTTTCGCCACCTCTGACTTGAGCGTCGATTT |
| | TTGTGATGCTCGTCAGGGGGGCGGAGCCTATGGAAAAACGCCAGCAACGCGGCCTTTTTA |
| | CGGTTCCTGGCCTTTTGCTGGCCTTTTGCTCACATGTTCTTTCCTGCGTTATCCCCTGAT |
| | TCTGTGGATAACCGTATTACCGCCTTTGAGTGAGCTGATACCGCTCGCCGCAGCCGAACG |
| | ACCGAGCGCAGCGAGTCAGTGAGCGAGGAAGCGGAAGAGCGCCTGATGCGGTATTTTCTC |
| | CTTACGCATCTGTGCGGTATTTCACACCGCAATGGTGCACTCTCAGTACAATCTGCTCTG |
| | ATGCCGCATAGTTAAGCCAGTATACACTCCGCTATCGCTACGTGACTGGGTCATGGCTGC |
| | GCCCCGACACCCGCCAACACCCGCTGACGCGCCCTGACGGGCTTGTCTGCTCCCGGCATC |
| | CGCTTACAGACAAGCTGTGACCGTCTCCGGGAGCTGCATGTGTCAGAGGTTTTCACCGTC |
| | ATCACCGAAACGCGCGAGGCAGCTGCGGTAAAGCTCATCAGCGTGGTCGTGAAGCGATTC |
| | ACAGATGTCTGCCTGTTCATCCGCGTCCAGCTCGTTGAGTTTCTCCAGAAGCGTTAATGT |
| | CTGGCTTCTGATAAAGCGGGCCATGTTAAGGGCGGTTTTTTCCTGTTTGGTCACTGATGC |
| | CTCCGTGTAAGGGGGATTTCTGTTCATGGGGGTAATGATACCGATGAAACGAGAGAGGAT |
| | GCTCACGATACGGGTTACTGATGATGAACATGCCCGGTTACTGGAACGTTGTGAGGGTAA |
| | ACAACTGGCGGTATGGATGCGGCGGGACCAGAGAAAAATCACTCAGGGTCAATGCCAGCG |
| | CTTCGTTAATACAGATGTAGGTGTTCCACAGGGTAGCCAGCAGCATCCTGCGATGCAGAT |
| | CCGGAACATAATGGTGCAGGGCGCTGACTTCCGCGTTTCCAGACTTTACGAAACACGGAA |
| | ACCGAAGACCATTCATGTTGTTGCTCAGGTCGCAGACGTTTTGCAGCAGCAGTCGCTTCA |
| | CGTTCGCTCGCGTATCGGTGATTCATTCTGCTAACCAGTAAGGCAACCCCGCCAGCCTAG |
| | CCGGGTCCTCAACGACAGGAGCACGATCATGCGCACCCGTGGGGCCGCCATGCCGGCGAT |
| | AATGGCCTGCTTCTCGCCGAAACGTTTGGTGGCGGGACCAGTGACGAAGGCTTGAGCGAG |
| | GGCGTGCAAGATTCCGAATACCGCAAGCGACAGGCCGATCATCGTCGCGCTCCAGCGAAA |
| | GCGGTCCTCGCCGAAAATGACCCAGAGCGCTGCCGGCACCTGTCCTACGAGTTGCATGAT |
| | AAAGAAGACAGTCATAAGTGCGGCGACGATAGTCATGCCCCGCGCCCACCGGAAGGAGCT |
| | GACTGGGTTGAAGGCTCTCAAGGGCATCGGTCGAGATCCCGGTGCCTAATGAGTGAGCTA |
| | ACTTACATTAATTGCGTTGCGCTCACTGCCCGCTTTCCAGTCGGGAAACCTGTCGTGCCA |
| | GCTGCATTAATGAATCGGCCAACGCGCGGGGAGAGGCGGTTTGCGTATTGGGCGCCAGGG |
| | TGGTTTTTCTTTTCACCAGTGAGACGGGCAACAGCTGATTGCCCTTCACCGCCTGGCCCT |
| | GAGAGAGTTGCAGCAAGCGGTCCACGCTGGTTTGCCCCAGCAGGCGAAAATCCTGTTTGA |
| | TGGTGGTTAACGGCGGGATATAACATGAGCTGTCTTCGGTATCGTCGTATCCCACTACCG |
| | AGATATCCGCACCAACGCGCAGCCCGGACTCGGTAATGGCGCGCATTGCGCCCAGCGCCA |
| | TCTGATCGTTGGCAACCAGCATCGCAGTGGGAACGATGCCCTCATTCAGCATTTGCATGG |
| | TTTGTTGAAAACCGGACATGGCACTCCAGTCGCCTTCCCGTTCCGCTATCGGCTGAATTT |
| | GATTGCGAGTGAGATATTTATGCCAGCCAGCCAGACGCAGACGCGCCGAGACAGAACTTA |
| | ATGGGCCCGCTAACAGCGCGATTTGCTGGTGACCCAATGCGACCAGATGCTCCACGCCCA |
| | GTCGCGTACCGTCTTCATGGGAGAAAATAATACTGTTGATGGGTGTCTGGTCAGAGACAT |
| | CAAGAAATAACGCCGGAACATTAGTGCAGGCAGCTTCCACAGCAATGGCATCCTGGTCAT |
| | CCAGCGGATAGTTAATGATCAGCCCACTGACGCGTTGCGCGAGAAGATTGTGCACCGCCG |
| | CTTTACAGGCTTCGACGCCGCTTCGTTCTACCATCGACACCACCACGCTGGCACCCAGTT |
| | GATCGGCGCGAGATTTAATCGCCGCGACAATTTGCGACGGCGCGTGCAGGGCCAGACTGG |
| | AGGTGGCAACGCCAATCAGCAACGACTGTTTGCCCGCCAGTTGTTGTGCCACGCGGTTGG |
| | GAATGTAATTCAGCTCCGCCATCGCCGCTTCCACTTTTTCCCGCGTTTTCGCAGAAACGT |
| | GGCTGGCCTGGTTCACCACGCGGGAAACGGTCTGATAAGAGACACCGGCATACTCTGCGA |
| | CATCGTATAACGTTACTGGTTTCACATTCACCACCCTGAATTGACTCTCTTCCGGGCGCT |
| | ATCATGCCATACCGCGAAAGGTTTTGCGCCATTCGATGGTGTCCGGGATCTCGACGCTCT |
| | CCCTTATGCGACTCCTGCATTAGGAAGCAGCCCAGTAGTAGGTTGAGGCCGTTGAGCACC |
| | GCCGCCGCAAGGAATGGTGCATGCAAGGAGATGGCGCCCAACAGTCCCCCGGCCACGGGG |
| | CCTGCCACCATACCCACGCCGAAACAAGCGCTCATGAGCCCGAAGTGGCGAGCCCGATCT |
| | TCCCCATCGGTGATGTCGGCGATATAGGCGCCAGCAACCGCACCTGTGGCGCCGGTGATG |
| | CCGGCCACGATGCGTCCGGCGTAGAGGATCGAGATCTCGATCCCGCGAAATTAATACGAC |
| | TCACTATAGGGGAATTGTGAGCGGATAACAATTCCCCTCTAGAAATAATTTTGTTTAACT |
| | TTAAGAAGGAGATATACCATGGGCAGCAGCCATCATCATCATCATCACAGCAGCGGCCTG |
| | GTGCCGCGCGGCAGCCATATGGCTAGCATGACTGGTGGACAGCAAATGGGTCGCGGATCC 7080 |
| 10 | TCCAAGATTTCCGAATCTATCTTCGTTGGTATCGCGGCCATCCCAGAGCGAGAAACGGCT |
| | CTTAAGCAAACGATTGCCTCACTTATAAACCAAGCTCCACGCATTGGTGTATATTTGAAC |
| | GGTTGGAAGCGTATTCCTGACTTCCTGCGCCACGAAAAGATCGAGATAGCGGGATTCCAC |
| | GACGCCGATCTTGGAGACGTCGGCAAGTTCCACTGGGTGGACAATCATGACGGCCTGTAC |
| | TTTACTTGCGACGACGACCTGGTATATCCCCCTGACTATATTAAGCGCACAGCAGCTAAA |
| | CTGAAGGAACACAACTACAGAGCCGCCGTCGGGTGGCACGGATCTTTAATCAAAGAGCCC |
| | TTTGTGAATTATTACGATGGAGCGTCCAGACGTGTGTTCGTGTTCAGCGCGCATCGGCCA |
| | GTGGACACTCCGGTGCACATACTGGGAACGGGTTGTTGTGCGTTCCACACGAGAGAATTA |
| | AGCATAAAGAAAAGTGATTTTGTCCATCCGAATATGGCTGACATATTCTTCTCAATCAAG |
| | GGTCAAGAGCAGAACATTCCTTTTATCGTGATCAAGCACGAGAAGAACGAGATCACAGAG |
| | GTAGAAGGCTCGAAGGAGTCAAGTATATTTGCACACAGCCATACAGATCAAGCCTCTAAG |
| | AAGAACACCAAGGAGATACAAAATACTTTTGTCTCAAATAACTTACCATGGGTACTGAAG |
| | TCCTTTCAACCCTTCAGCATATTAATTATCGGTCGCTTTGAGAGCTACAGCAAGGGTGGA |
| | ATATACAAGTCCTGTCATTTAATTAAGAAACACCTCACGGAGCTGGGACATAATGTTTCC |
| | ATCTTAGATACTGGGAACGAGATGACGGCTGAGCATGTGAGCGGGCACGATATCTGTTGG |
| | ATATACCCCGGTGACCCTGAACGCCCGGACTTCAGCACTGTAGACGACAAGATTCACTTG |
| | CTTCGCAAGCACGGTGTGCCTGTATTGCTTAACTTATCCTACTTATATGAATCACACAGA |
| | TCCCAGTGGATAGTAGAAAAGTTGAAGGAGTACAACAGCCAAGCGGGTACACCAGTATTA |
| | GCAGCTGTGTTCACCGAAAGCGCAGCAAATGACCCGTTGCTTAATAGTGTTCGCGACTAC |
| | GTATGCGTTGTTCCTAAGACCTTGCTCCCAACTCCATACGAAATGGTTCCCTCTTTCGAC |
| | CAACGTGAAGGAATCTGTTTAGGAGATGCCACTAAGTTAGGCAATCCTATAGTAATCGGT |
| | GGGAGTATAAACCCGTGGATCGACGCTATTCACAACCGTCTGCCACACGTAAACATCTAC |
| | GCCTACAAGCAATATCAAGGCAACAACCCGCATCCAAAGGTCAAGTACGTGAGTCATATG |
| | AAGGAGAATTTTGGCGAGTGGCTGGCCCAACGGCGTCTGTTCATCTGCGCCAATGTCCAC |
| | CTCACATTCGAGATGGTTGCATGTGAGGCACAGCAGTACGGTACCCCAACACTGTACCGC |
| | CATATGCCCCACTCATTATCCGAGTATATCTCAGCAACTGGCATGGCCGTACGTACCCCC |
| | GGTGAAATGGGCGAAATGGCGGCGTGGCTGTACAACTCACGGGCCACCTGGGACAAGTTA |
| | TCAGCATCATCTATACACAACGCCAATAGTAAACACGTGGATCTTTTGGACGCTAGCCTT |
| | GAAGGATACATAAGACTCGCTATTTACCGTGCATCTCTCCTGAAGAGTAAATTCTTGACT |
| | AAGAAGTAAAAGCTTGCGGCCGCACTCGAGCACCACCACCACCACCACTGAGATCCGGCT |
| | GCTAACAAAGCCCGAAAGGAAGCTGAGTTGGCTGCTGCCACCGCTGAGCAATAACTAGCA |
| | TAACCCCTTGGGGCCTCTAAACGGGTCTTGAGGGGTTTTTTGCTGAAAGGAGGAACTATA |
| | TCCGGATTGGCGAATGGGACGCGCCCTGTAGCGGCGCATTAAGCGCGGCGGGTGTGGTGG |
| | TTACGCGCAGCGTGACCGCTACACTTGCCAGCGCCCTAGCGCCCGCTCCTTTCGCTTTCT |
| | TCCCTTCCTTTCTCGCCACGTTCGCCGGCTTTCCCCGTCAAGCTCTAAATCGGGGGCTCC |
| | CTTTAGGGTTCCGATTTAGTGCTTTACGGCACCTCGACCCCAAAAAACTTGATTAGGGTG |
| | ATGGTTCACGTAGTGGGCCATCGCCCTGATAGACGGTTTTTCGCCCTTTGACGTTGGAGT |
| | CCACGTTCTTTAATAGTGGACTCTTGTTCCAAACTGGAACAACACTCAACCCTATCTCGG |
| | TCTATTCTTTTGATTTATAAGGGATTTTGCCGATTTCGGCCTATTGGTTAAAAAATGAGC |
| | TGATTTAACAAAAATTTAACGCGAATTTTAACAAAATATTAACGCTTACAATTTAGGTGG |
| | CACTTTTCGGGGAAATGTGCGCGGAACCCCTATTTGTTTATTTTTCTAAATACATTCAAA |
| | TATGTATCCGCTCATGAATTAATTCTTAGAAAAACTCATCGAGCATCAAATGAAACTGCA |
| | ATTTATTCATATCAGGATTATCAATACCATATTTTTGAAAAAGCCGTTTCTGTAATGAAG |
| | GAGAAAACTCACCGAGGCAGTTCCATAGGATGGCAAGATCCTGGTATCGGTCTGCGATTC |
| | CGACTCGTCCAACATCAATACAACCTATTAATTTCCCCTCGTCAAAAATAAGGTTATCAA |
| | GTGAGAAATCACCATGAGTGACGACTGAATCCGGTGAGAATGGCAAAAGTTTATGCATTT |
| | CTTTCCAGACTTGTTCAACAGGCCAGCCATTACGCTCGTCATCAAAATCACTCGCATCAA |
| | CCAAACCGTTATTCATTCGTGATTGCGCCTGAGCGAGACGAAATACGCGATCGCTGTTAA |
| | AAGGACAATTACAAACAGGAATCGAATGCAACCGGCGCAGGAACACTGCCAGCGCATCAA |
| | CAATATTTTCACCTGAATCAGGATATTCTTCTAATACCTGGAATGCTGTTTTCCCGGGGA |
| | TCGCAGTGGTGAGTAACCATGCATCATCAGGAGTACGGATAAAATGCTTGATGGTCGGAA |
| | GAGGCATAAATTCCGTCAGCCAGTTTAGTCTGACCATCTCATCTGTAACATCATTGGCAA |
| | CGCTACCTTTGCCATGTTTCAGAAACAACTCTGGCGCATCGGGCTTCCCATACAATCGAT |
| | AGATTGTCGCACCTGATTGCCCGACATTATCGCGAGCCCATTTATACCCATATAAATCAG |
| | CATCCATGTTGGAATTTAATCGCGGCCTAGAGCAAGACGTTTCCCGTTGAATATGGCTCA |
| | TAACACCCCTTGTATTACTGTTTATGTAAGCAGACAGTTTTATTGTTCATGACCAAAATC |
| | CCTTAACGTGAGTTTTCGTTCCACTGAGCGTCAGACCCCGTAGAAAAGATCAAAGGATCT |
| | TCTTGAGATCCTTTTTTTCTGCGCGTAATCTGCTGCTTGCAAACAAAAAAACCACCGCTA |
| | CCAGCGGTGGTTTGTTTGCCGGATCAAGAGCTACCAACTCTTTTTCCGAAGGTAACTGGC |
| | TTCAGCAGAGCGCAGATACCAAATACTGTCCTTCTAGTGTAGCCGTAGTTAGGCCACCAC |
| | TTCAAGAACTCTGTAGCACCGCCTACATACCTCGCTCTGCTAATCCTGTTACCAGTGGCT |
| | GCTGCCAGTGGCGATAAGTCGTGTCTTACCGGGTTGGACTCAAGACGATAGTTACCGGAT |
| | AAGGCGCAGCGGTCGGGCTGAACGGGGGGTTCGTGCACACAGCCCAGCTTGGAGCGAACG |
| | ACCTACACCGAACTGAGATACCTACAGCGTGAGCTATGAGAAAGCGCCACGCTTCCCGAA |
| | GGGAGAAAGGCGGACAGGTATCCGGTAAGCGGCAGGGTCGGAACAGGAGAGCGCACGAGG |
| | GAGCTTCCAGGGGGAAACGCCTGGTATCTTTATAGTCCTGTCGGGTTTCGCCACCTCTGA |
| | CTTGAGCGTCGATTTTTGTGATGCTCGTCAGGGGGGCGGAGCCTATGGAAAAACGCCAGC |
| | AACGCGGCCTTTTTACGGTTCCTGGCCTTTTGCTGGCCTTTTGCTCACATGTTCTTTCCT |
| | GCGTTATCCCCTGATTCTGTGGATAACCGTATTACCGCCTTTGAGTGAGCTGATACCGCT |
| | CGCCGCAGCCGAACGACCGAGCGCAGCGAGTCAGTGAGCGAGGAAGCGGAAGAGCGCCTG |
| | ATGCGGTATTTTCTCCTTACGCATCTGTGCGGTATTTCACACCGCAATGGTGCACTCTCA |
| | GTACAATCTGCTCTGATGCCGCATAGTTAAGCCAGTATACACTCCGCTATCGCTACGTGA |
| | CTGGGTCATGGCTGCGCCCCGACACCCGCCAACACCCGCTGACGCGCCCTGACGGGCTTG |
| | TCTGCTCCCGGCATCCGCTTACAGACAAGCTGTGACCGTCTCCGGGAGCTGCATGTGTCA |
| | GAGGTTTTCACCGTCATCACCGAAACGCGCGAGGCAGCTGCGGTAAAGCTCATCAGCGTG |
| | GTCGTGAAGCGATTCACAGATGTCTGCCTGTTCATCCGCGTCCAGCTCGTTGAGTTTCTC |
| | CAGAAGCGTTAATGTCTGGCTTCTGATAAAGCGGGCCATGTTAAGGGCGGTTTTTTCCTG |
| | TTTGGTCACTGATGCCTCCGTGTAAGGGGGATTTCTGTTCATGGGGGTAATGATACCGAT |
| | GAAACGAGAGAGGATGCTCACGATACGGGTTACTGATGATGAACATGCCCGGTTACTGGA |
| | ACGTTGTGAGGGTAAACAACTGGCGGTATGGATGCGGCGGGACCAGAGAAAAATCACTCA |
| | GGGTCAATGCCAGCGCTTCGTTAATACAGATGTAGGTGTTCCACAGGGTAGCCAGCAGCA |
| | TCCTGCGATGCAGATCCGGAACATAATGGTGCAGGGCGCTGACTTCCGCGTTTCCAGACT |
| | TTACGAAACACGGAAACCGAAGACCATTCATGTTGTTGCTCAGGTCGCAGACGTTTTGCA |
| | GCAGCAGTCGCTTCACGTTCGCTCGCGTATCGGTGATTCATTCTGCTAACCAGTAAGGCA |
| | ACCCCGCCAGCCTAGCCGGGTCCTCAACGACAGGAGCACGATCATGCGCACCCGTGGGGC |
| | CGCCATGCCGGCGATAATGGCCTGCTTCTCGCCGAAACGTTTGGTGGCGGGACCAGTGAC |
| | GAAGGCTTGAGCGAGGGCGTGCAAGATTCCGAATACCGCAAGCGACAGGCCGATCATCGT |
| | CGCGCTCCAGCGAAAGCGGTCCTCGCCGAAAATGACCCAGAGCGCTGCCGGCACCTGTCC |
| | TACGAGTTGCATGATAAAGAAGACAGTCATAAGTGCGGCGACGATAGTCATGCCCCGCGC |
| | CCACCGGAAGGAGCTGACTGGGTTGAAGGCTCTCAAGGGCATCGGTCGAGATCCCGGTGC |
| | CTAATGAGTGAGCTAACTTACATTAATTGCGTTGCGCTCACTGCCCGCTTTCCAGTCGGG |
| | AAACCTGTCGTGCCAGCTGCATTAATGAATCGGCCAACGCGCGGGGAGAGGCGGTTTGCG |
| | TATTGGGCGCCAGGGTGGTTTTTCTTTTCACCAGTGAGACGGGCAACAGCTGATTGCCCT |
| | TCACCGCCTGGCCCTGAGAGAGTTGCAGCAAGCGGTCCACGCTGGTTTGCCCCAGCAGGC |
| | GAAAATCCTGTTTGATGGTGGTTAACGGGGGGATATAACATGAGCTGTCTTCGGTATCGT |
| | CGTATCCCACTACCGAGATATCCGCACCAACGCGCAGCCCGGACTCGGTAATGGCGCGCA |
| | TTGCGCCCAGCGCCATCTGATCGTTGGCAACCAGCATCGCAGTGGGAACGATGCCCTCAT |
| | TCAGCATTTGCATGGTTTGTTGAAAACCGGACATGGCACTCCAGTCGCCTTCCCGTTCCG |
| | CTATCGGCTGAATTTGATTGCGAGTGAGATATTTATGCCAGCCAGCCAGACGCAGACGCG |
| | CCGAGACAGAACTTAATGGGCCCGCTAACAGCGCGATTTGCTGGTGACCCAATGCGACCA |
| | GATGCTCCACGCCCAGTCGCGTACCGTCTTCATGGGAGAAAATAATACTGTTGATGGGTG |
| | TCTGGTCAGAGACATCAAGAAATAACGCCGGAACATTAGTGCAGGCAGCTTCCACAGCAA |
| | TGGCATCCTGGTCATCCAGCGGATAGTTAATGATCAGCCCACTGACGCGTTGCGCGAGAA |
| | GATTGTGCACCGCCGCTTTACAGGCTTCGACGCCGCTTCGTTCTACCATCGACACCACCA |
| | CGCTGGCACCCAGTTGATCGGCGCGAGATTTAATCGCCGCGACAATTTGCGACGGCGCGT |
| | GCAGGGCCAGACTGGAGGTGGCAACGCCAATCAGCAACGACTGTTTGCCCGCCAGTTGTT |
| | GTGCCACGCGGTTGGGAATGTAATTCAGCTCCGCCATCGCCGCTTCCACTTTTTCCCGCG |
| | TTTTCGCAGAAACGTGGCTGGCCTGGTTCACCACGCGGGAAACGGTCTGATAAGAGACAC |
| | CGGCATACTCTGCGACATCGTATAACGTTACTGGTTTCACATTCACCACCCTGAATTGAC |
| | TCTCTTCCGGGCGCTATCATGCCATACCGCGAAAGGTTTTGCGCCATTCGATGGTGTCCG |
| | GGATCTCGACGCTCTCCCTTATGCGACTCCTGCATTAGGAAGCAGCCCAGTAGTAGGTTG |
| | AGGCCGTTGAGCACCGCCGCCGCAAGGAATGGTGCATGCAAGGAGATGGCGCCCAACAGT |
| | CCCCCGGCCACGGGGCCTGCCACCATACCCACGCCGAAACAAGCGCTCATGAGCCCGAAG |
| | TGGCGAGCCCGATCTTCCCCATCGGTGATGTCGGCGATATAGGCGCCAGCAACCGCACCT |
| | GTGGCGCCGGTGATGCCGGCCACGATGCGTCCGGCGTAGAGGATCGAGATCTCGATCCCG |
| | CGAAATTAATACGACTCACTATAGGGGAATTGTGAGCGGATAACAATTCCCCTCTAGAAA |
| | TAATTTTGTTTAACTTTAAGAAGGAGATATACCATGGGCAGCAGCCATCATCATCATCAT |
| | CACAGCAGCGGCCTGGTGCCGCGCGGCAGCCATATGGCTAGCATGACTGGTGGACAGCAA |
| | ATGGGTCGCGGATCC 7095 |
| 14 | MXXXXXXXXX XXXXXXXXXX AXXXFXXXXX XXAXXXYXXX JXXXPXXXXX XXXNJXJXXX 60 |
| | XXXXXXXXXX XXXXXXXXXX XXXFVGXAAI PXRXXALXXX IXSLLXQXXX IGVYLNGWXX 120 |
| | XPXXLXXEKI XXXGXXXXDJ GDVGKFXWVD XHDGXYFXCD DDLXYPXDYX XRTXXKLXXX 180 |
| | XXXAAXGWHG SXLXXXFXXY YDXXSRXVFX FXXHRPXDTX VHILGTGCXA FHTXXXXXXK 240 |
| | XDFXXPNMAD IFFXJXGQXQ XIPFXVIXHE KXXIXEXXGX KESSIXXXSX XNXXXXKNTX 300 |
| | XLQNXXVXXN XPWXXXXXQX XSXLIXGRFX XXXKGGIYKS CHLIXXHLXX LGHXVXXXDT 360 |
| | XXXXXXXXXX XXDLCWIYPG DPERPDFXXV XXKIXXLXXX GXPVLXNLSY LYXXXRXXWI 420 |
| | XXXIXXXNXX XXTPVLXAVF TEXAAXDPLL XXVRDYXCVV PKTIXPXXXX XXXXFXEREG 480 |
| | XCLGDATKLX NXXXIGGXXX XWIDAIXXXX PXVNLYAXKQ YQGBNPHXXX XYXXHMKEXF 540 |
| | GXWLXQRRLF JCXNXHXTFE MVXCEAQXXG TPXLYRHMPH SLSEYISXTG XAXRXPXEMX 600 |
| | EMXXWLYNXX XXWXXXSXXS LXNXXXXXVD XLDXSLEGYL RLAXXRXXXX XXXXXXXXXX 660 |
| | XX 662 |
| 15 | SIFVGIAAIP ERXXALXXTI XSLJXQXXXI GVYLNGWKXX PDXLXXEKIX XXGFXXXDLG 60 |
| | DVGKFXWVDX HDGJYFXCDD DLXYPXDYXX RTXXKLKEXN YXAAXGWHGS LJXXXFXXYY 120 |
| | DXXSRRVFVF SAHRPXDTPV HILGTGCXAF HTXXLXIKKS DFXHPNMADI FFSIKGQEQX 180 |
| | IPFIVJXHEK BEITEXXGXK ESSIXXHSXX BXXSKKNTXX JQNXFVXXNX PWVXXXXXXX 240 |
| | SXLIXGRFEX YSKGGIYKSC HLIKXHLXXL GHBVXIXDTX NXXXXXXXXX XDJCWIYPGD 300 |
| | PERPDFSXVX DKIXXLXXXG XPVJXNLSYL YXXXRXXWIX XKJXXXNXXX XTPVLXAVFT 360 |
| | EXAANDPLLX XVRDYXCVVP KTJLPTPXEX XXXFXZREGI CLGDATKLGN XXVIGGXXNX 420 |
| | WIDAIHNRLP HVNJYAYKQY QGNNPHPKXK YXXHMKENFG XWLAQRRJFI CXNVHLTFEM 480 |
| | VACEAQXYGT PXJYRHMPHS LSEYISATGX AXRXPXEMXE MXAWLYNXXX XWBKXSXXSJ 540 |
| | XNXXXXXVBL LDXSLEGYJR LAIXRXXXXX XK 572 |
| 16 | MXXXXXXXXX XXXXXXXXXX AXXXFXXXXX XXAXXXYXXX JXXXPXXXXX XXXNJXJXXX 60 |
| | XXXXXXXXXX XXXXXXXXXX XXXFVGXAAI PXRXXALXXX IXSLLXQXXX IGVYLNGWXX 120 |
| | XPXXLXXEKI XXXGXXXXDJ GDVGKFXWVD XHDGXYFXCD DDLXYPXDYX XRTXXKLXXX 180 |
| | XXXAAXGWHG SXLXXXFXXY YDXXSRXVFX FXXHRPXDTX VHILGTGCXA FHTXXXXXXK 240 |
| | XDFXXPNMAD IFFXJXGQXQ XIPFXVIXHE KXXIXEXXGX KESSIXXXSX XNXXXXKNTX 300 |
| | XLQNXXVXXN XPWXXXXXQX XSXLIXGRFX XXXKGGIYKS CHLIXXHLXX LGHXVXXXDT 360 |
| | XXXXXXXXXX XXDLCWIYPG DPERPDFXXV XXKIXXLXXX GXPVLXNLSY LYXXXRXXWI 420 |
| | XXXIXXXNXX XXTPVLXAVF TEXAAXDPLL XXVRDYXCVV PKTIXPXXXX XXXXFXERE 480 |
| | XCLGDATKLX NXXXIGGXXX XWIDAIXXXX PXVNLYAXKQ YQGBNPHXXX XYXXHMKEXF 540 |
| | GXWLXQRRLF JCXNXHXTFE MVXCEAQXXG TPXLYRHMPH SLSEYISXTG XAXRXPXEMX 600 |
| | EMXXWLYNXX XXWXXXSXXS LXNXXXXXVD XLDXSLEGYL RLAXXRXXXX XXXXXXXXXX 660 |
| | XX 662 |

Note that in the specification and claims, “about” or “approximately” means within twenty percent (20%) of the numerical amount cited. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

Although embodiments of the invention have been described in detail with particular reference to these embodiments, other embodiments can achieve the same results. Variations and modifications of embodiments of the present invention will be obvious to those skilled in the art and it is intended to cover in the appended claims all such modifications and equivalents. The entire disclosures of all references, applications, patents, and publications cited above are hereby incorporated by reference.

1. A recombinant microbial system for producing Testosteronan (“Testan”), comprising:
(a) a microbial host cell;
(b) a nucleic acid sequence encoding an enzymatically active Testan synthase wherein the nucleotide sequence is defined as:
(iii) a nucleotide sequence encoding the amino acid sequence of SEQ ID NO:6 and a truncated SEQ ID NO. 6 wherein the nucleotide sequence encoding the N-terminal amino acids selected from 1-50, 1-60, 1-61, 1-62, 1-63, 1-64, 1-66, 1-67,1-68, 1-69 or 1-70 of SEQ ID NO:6 are deleted;
(v) a nucleotide sequence encoding the amino acid sequence of SEQ ID NO:7 or an amino acid sequence having at least about 85% sequence identify to SEQ ID NO: 7;
(c) an expression construct comprising a heterologous promoter operably linked to one of the nucleic acid sequences of (b);
wherein the system produces a polymer at least a portion of which has the repeat structure [→4)-α-D-GlcA-(1→4)-α-D-GlcNAc-(1→]ₙ, wherein n is a positive integer greater than 0, and the polymer having a number-average molecular weight of between about 100 kDa to about 2 mDa.

2. (canceled)

3. The system of claim 1, wherein enzymatically active Testan synthase has a Testan synthase polymerizing activity about 50% or greater as compared to the Testan synthase polymerizing activity of SEQ ID NO:7 under similar polymerizing conditions.

4. The system of claim 1, wherein the Testan synthase has a deletion of amino acids at the N-terminal portion of SEQ ID NO:6 and retains at least 90% polymerizing activity.

5. The system of claim 1, wherein the microbial host cell is selected from Escherichia coli, Bacillus subtilis, Bacillus megaterium, or Pseudomonas species.

6. The system of claim 1, wherein the nucleic acid encoding the Testan synthase is codon-optimized for expression in the host cell.

7. The system of claim 1, wherein the promoter is selected from T7, lac, arabinose, CMV, BioBrick, or constitutive promoters.

8. The system of claim 1, wherein the nucleic acid is present on a multi-copy plasmid or integrated into the host genome.

9. The system of claim 1, wherein the host cell further expresses one or more enzymes selected from UDP-glucose dehydrogenase, UDP-GlcNAc pyrophosphorylase, GlmU, GlmM, GlmS, Pgi, GalU, or Ugd.

10. The system of claim 1, wherein the host is a GRAS organism and the Testan polymer recovered is substantially endotoxin-free.

11. The system of claim 1, wherein the Testan polymer exhibits a polydispersity index less than 1.5.

12. The system of claim 1, wherein the Testan polymer exhibits a molecular weight between about 800 kDa and 1200 kDa.

13. A method for producing high-molecular-weight Testosteronan (“Testan”), comprising:
(a) culturing a recombinant host cell expressing a Testan synthase polypeptide of SEQ ID NO:6, SEQ ID NO:7 or a polypeptide having at least 85% sequence identity to SEQ ID NO:6 or SEQ ID NO:7;
(b) inducing expression of the Testan synthase under a heterologous promoter to produce a Testan polymer;
(c) allowing the Testan polymer to accumulate in a culture medium; and
(d) isolating the Testan polymer from the culture medium,
wherein the isolated Testan polymer has a number-average molecular weight of between about 100 kDa to about 2 mDa.

14. The method of claim 13, wherein the culturing comprises fed-batch, batch-fed, or continuous fermentation.

15. The method of claim 13, wherein the host cell co-expresses UDP-sugar biosynthetic enzymes to enhance precursor flux.

16. The method of claim 13, wherein the culture medium comprises defined minimal media supplemented with glycerol and ammonium chloride.

17. The method of claim 13, wherein the method is performed under cGMP-compatible fermentation conditions.

18. The method of claim 13, further comprising purifying the Testan polymer using ultrafiltration, diafiltration, anion-exchange chromatography, or combinations thereof.

19. The method of claim 13, wherein the purified Testan polymer comprises at least 95% Testan by weight.

20. The method of claim 13, wherein the purified Testan polymer is between about 800 kDa and about 1200 kDa.

21-80. (canceled)

81. A method for separating a quantity of a compound using chromatography, comprising:
providing a compound mixture to a chromatography environment comprising a chromatography substrate functionalized with a Testosteronan polymer; and
passing the compound mixture past the chromatography substrate to elute a selected eluate from the chromatography environment.

82. The method of claim 81 wherein the Testosteronan polymer is coated to a surface of the substrate as a stationary phase (SP), where the polysaccharide is physically adsorbed onto the surface of the substrate or covalently bonded as a SP wherein the polysaccharide is grafted onto the surface of the substrate.

83. The method of claim 81 wherein the substrate is selected from silica, silicon, semiconductors, glass, polymers, metals, gold, copper, stainless steel, nickel, aluminum, titanium, thermosensitive alloys and combinations thereof.

84. The method of claim 81 wherein the chromatography environment is selected from thin layer chromatography, liquid chromatography, and membrane chromatography.


SEQUENCE LISTING (16 sequences)

SEQ ID NO: 1 - protein, 640 aa, Comamonas testosteroni
  source 1..640: note = strain is KF-1
MSGMFKVANDFFSNGNFEKAIERYEEIIFKYPGLTEFASGNLALARRKLGERQENKSKSLVNASKISESIFVGIAAIPERAKALEKTIESLLPQVEKIGVYLNGWKEVPDYLKNEKILVEGFGKEDLGDVGKFFWVDQHDGIYFSCDDDLIYPKDYVDRTVEKLKEKNYKAAIGWHGSLLRDNFSTYYDKNSRRVFVFSAHRPWDTPVHILGTGCSAFHTKFLKIKKSDFLHPNMADIFFSIKGQEQKIPFIVLAHEKDEITEFVGAKESSIYSHSQANVESKKNTHDLQNGFVMKNMPWVMNDVESLSVLIVGRFENYSKGGIYKSCHLIKEHLSALGHDVDIHDTQNPFAKALEKKYDLCWIYPGDPERPDFSSVEDKIYELKSRGIPVIVNLSYLYSEDRTIWIRNKIRDLNAKGTTPVLGAVFTETAANDPLLKDVRDYICVVPKTILPTPCERYYEFGEREGICLGDATKLGNAKVIGGNVNNWIDAIHNRLPHVNLYAYKQYQGNNPHPKIKYAPHMKENFGDWLAQRRIFICLNVHLTFEMVACEAQSYGTPVIYRHMPHSLSEYISATGFATRSPDEMAEMVAWLYNNNKAWNKMSQASLNNGKANNVNLLDSSLEGYLRLAILRIKKMMVK

SEQ ID NO: 2 - DNA, 1923 bp, Comamonas testosteroni
  source 1..1923: mol_type = genomic DNA; strain = KF-1
atgagtggaatgtttaaggttgccaatgattttttctctaacggtaattttgaaaaagcaattgaacgttatgaggaaataatttttaagtatccaggcttgacagagtttgcgtcagggaatttagcgctggcaagaagaaaactaggagagcgccaagaaaataaatctaaatcattggtgaatgcttccaagatatctgagagtatttttgtaggtatagcagctataccagaacgcgcgaaagctttagagaaaactattgagtctttattgcctcaggtagaaaaaataggggtttacttaaacggttggaaggaagtcccagattacttgaagaatgagaaaatccttgtagagggattcggcaaagaagacctgggagacgtgggtaagtttttttgggtggatcagcatgatggaatctatttctcatgtgatgacgacttaatctatccaaaggactatgttgatagaacagttgaaaagttaaaagagaaaaattacaaagctgcaattggctggcatggttctctattgagagataattttagtacttattacgataagaattctcgacgtgtttttgttttttctgcacatcgtccatgggatacgcctgtacatattctaggtacaggatgctcagcgttccatactaagttcttaaagataaagaagtctgattttctgcatccaaatatggcggatatatttttctccattaaagggcaggaacaaaaaataccttttattgtcttggcgcatgaaaaagatgaaataacagagtttgttggggctaaggaaagctctatttactcgcattctcaggcgaatgtggaatcgaaaaaaaatacccatgatttgcaaaatggttttgtaatgaaaaacatgccatgggtcatgaatgatgtcgagtctctatctgttttgatagttggtagatttgaaaattatagtaaaggtgggatctataagtcatgccatcttattaaagagcatctgtcggcactgggtcatgatgttgatattcatgatactcaaaatccattcgccaaggcgttagagaaaaaatatgatctgtgttggatatacccaggtgatcctgagcgaccagatttttctagtgttgaagataaaatttatgaactgaagtcaaggggtattccggtaatcgttaatctctcttatttgtattctgaagatcgaaccatatggattcgaaataaaatacgagatttgaatgcgaaaggaactactcctgtacttggagctgtgtttacagaaactgccgcaaatgatccgctgctaaaagatgtgagagactatatttgcgtcgttcctaagacgatactacctacaccttgtgaaaggtattatgagtttggtgagcgagagggtatatgtttgggggatgcaacgaaactcgggaatgctaaagttataggcggtaatgttaataattggattgatgcaattcacaatagacttcctcatgtcaatctttatgcatacaagcagtatcaaggtaataacccacatccaaagattaaatatgctccacatatgaaagagaattttggtgattggttggcgcaacgtaggatatttatctgtttgaacgttcatttaacatttgagatggtggcgtgcgaggcgcaaagttacggtacacctgtgatttatagacatatgccgcactccttgagtgagtatatctctgctacggggtttgcaactcggtcacctgatgaaatggctgaaatggtggcatggttatataataataacaaggcgtggaataagatgagtcaagcttctctaaataatgggaaggctaataatgttaatttattagactcctctcttgaggggtatttgagattggctattctcagaattaaaaagatgatggtgaaatga

SEQ ID NO: 3 - protein, 662 aa, Candidatus Symbiobacter mobilis
MQSQNNHPPAQPGTQAQVRHAYALFKQGRYPEAMEAYERILASHPEMERRLRVNIHIAKKRIQNGIRTRQTLARDDKELSNAVFVGMAAIPDRVAALEASICSLLPQAERIGVYLNGWEQIPSFLQHEKIIVAGMGEPDIGDVGKFHWVDDHDGYYFTCDDDLIYPPDYIARTLAKLRHYHGNAAVGWHGSVLMEPFARYYDTNSRKVFLFGSHRPHDTAVHILGTGCCAFHTQVMQVRKADFTAPNMADIFFALNGQKQKIPFYVIEHEKGAIREAPGTKESSIYADSHQNKSTRKNTHELQNQLVRSNAPWLLHEFQPLSILIIGRFNSFQKGGIYKSCHLIQEHLSNLGHHVSVLDTQHALVLPESAHFDLCWIYPGDPERPDFATVVEKIRSLQKRGIPVLVNLSYLYQEKRTTWIRDRILECNAGQSTPVLCAVFTESAAHDPLLADVRDYVCVVPKTIEPPACTKVRGFGEREGVCLGDATKLSNPKIIGGPVHAWIDAIFRKMPYVNLYAFKQYQGDNPHSRVQYVPHMKEEFGDWLGQRRLFLCLNIHTTFEMVSCEAQYCGTPVLYRHMPHSLSEYISTTGMAVRNPEEMAEMVTWLYNDPAAWERCSRSSLRNAESKHVDVLDASLEGYLRLAVQRARRLTKPATSTSDQRC

SEQ ID NO: 4 - protein, 638 aa, Alteromonas pelagimontana
MSLKEANSLVRQDKLLEAKKIYNKIYKSSSPTIREQIEFNLKLINKKLLNNGNHKSFSVHLSFEEPTFVGIASIPSREYALLETVKSLVKQVDKIGIFLDGYSEIPDFLKDNKKILIKRSQDFDRKVGDAGKFFWVDEHKGFYFTCDDDLIYPNDYVARIKQKIISKREPVVVGWHGSLILCPFKNYYNKNSRRVFTFGSARPEDTPVHILGTGCVGFHTSSFNVNFCDFPTPNMADVYFAKLGQEQKVPFIVIKHEKEEIQEVPDTREVSIYQHSHSNKDTEHNTKTTQNEVVSSIDWNVNYLKNSLDILIVGRFLINEKGGIFKSSRLLVEGLTQLGHRVSTCCLSQIDEFDFSSKKFDFSIIYAPDPERPDFSNCIDKVQLMIRLGVVCAVNFSFNLNENRTRWIERKLTELNSPFSYPRCFFASFSNSTSALFSEDISSMIVPFPKTISLNLKGKERLRYSEREGIFLGDLAKLLDEKLTHGNLLPWIEELSKRLPHVNLYAIKHYHTDLDYPKNIKVIPYTKNIENVLGNYRLCINLTPGATFEMVPLEALLSGTPVLHRRMPQSLSEYLSPVSIEINSPKELGEVCQRIYENENIWNRLSKAGSGSYEFFRIENITAALDLSIRKCLNRSGN

SEQ ID NO: 5 - protein, 572 aa, synthetic construct
  source 1..572: note = truncated Comamonas testosteroni KF1
SIFVGIAAIPERAKALEKTIESLLPQVEKIGVYLNGWKEVPDYLKNEKILVEGFGKEDLGDVGKFFWVDQHDGIYFSCDDDLIYPKDYVDRTVEKLKEKNYKAAIGWHGSLLRDNFSTYYDKNSRRVFVFSAHRPWDTPVHILGTGCSAFHTKFLKIKKSDFLHPNMADIFFSIKGQEQKIPFIVLAHEKDEITEFVGAKESSIYSHSQANVESKKNTHDLQNGFVMKNMPWVMNDVESLSVLIVGRFENYSKGGIYKSCHLIKEHLSALGHDVDIHDTQNPFAKALEKKYDLCWIYPGDPERPDFSSVEDKIYELKSRGIPVIVNLSYLYSEDRTIWIRNKIRDLNAKGTTPVLGAVFTETAANDPLLKDVRDYICVVPKTILPTPCERYYEFGEREGICLGDATKLGNAKVIGGNVNNWIDAIHNRLPHVNLYAYKQYQGNNPHPKIKYAPHMKENFGDWLAQRRIFICLNVHLTFEMVACEAQSYGTPVIYRHMPHSLSEYISATGFATRSPDEMAEMVAWLYNNNKAWNKMSQASLNNGKANNVNLLDSSLEGYLRLAILRIKKMMVK

SEQ ID NO: 6 - protein, 647 aa, Pseudomonas sp.
  source 1..647: note = strain: CIP-10
MDKLNTPSNLATANQLFRAKKYSEAALFYKKSIQESPELAEAIRINLHLCAKRAPAGEIEQITFQNNNEDNIFVGIAAIPERETALKQTIASLINQAPRIGVYLNGWKRIPDFLRHEKIEIAGFHDADLGDVGKFHWVDNHDGLYFTCDDDLVYPPDYIKRTAAKLKEHNYRAAVGWHGSLIKEPFVNYYDGASRRVFVFSAHRPVDTPVHILGTGCCAFHTRELSIKKSDFVHPNMADIFFSIKGQEQNIPFIVIKHEKNEITEVEGSKESSIFAHSHTDQASKKNTKEIQNTFVSNNLPWVLKSFQPFSILIIGRFESYSKGGIYKSCHLIKKHLTELGHNVSILDTGNEMTAEHVSGHDICWIYPGDPERPDFSTVDDKIHLLRKHGVPVLLNLSYLYESHRSQWIVEKLKEYNSQAGTPVLAAVFTESAANDPLLNSVRDYVCVVPKTLLPTPYEMVPSFDQREGICLGDATKLGNPIVIGGSINPWIDAIHNRLPHVNIYAYKQYQGNNPHPKVKYVSHMKENFGEWLAQRRLFICANVHLTFEMVACEAQQYGTPTLYRHMPHSLSEYISATGMAVRTPGEMGEMAAWLYNSRATWDKLSASSIHNANSKHVDLLDASLEGYIRLAIYRASLLKSKFLTKK

SEQ ID NO: 7 - protein, 572 aa, Synthetic construct
  source 1..572: note = Pseudomonas sp. CIP10
SIFVGIAAIPERETALKQTIASLINQAPRIGVYLNGWKRIPDFLRHEKIEIAGFHDADLGDVGKFHWVDNHDGLYFTCDDDLVYPPDYIKRTAAKLKEHNYRAAVGWHGSLIKEPFVNYYDGASRRVFVFSAHRPVDTPVHILGTGCCAFHTRELSIKKSDFVHPNMADIFFSIKGQEQNIPFIVIKHEKNEITEVEGSKESSIFAHSHTDQASKKNTKEIQNTFVSNNLPWVLKSFQPFSILIIGRFESYSKGGIYKSCHLIKKHLTELGHNVSILDTGNEMTAEHVSGHDICWIYPGDPERPDFSTVDDKIHLLRKHGVPVLLNLSYLYESHRSQWIVEKLKEYNSQAGTPVLAAVFTESAANDPLLNSVRDYVCVVPKTLLPTPYEMVPSFDQREGICLGDATKLGNPIVIGGSINPWIDAIHNRLPHVNIYAYKQYQGNNPHPKVKYVSHMKENFGEWLAQRRLFICANVHLTFEMVACEAQQYGTPTLYRHMPHSLSEYISATGMAVRTPGEMGEMAAWLYNSRATWDKLSASSIHNANSKHVDLLDASLEGYIRLAIYRASLLKSK

SEQ ID NO: 8 - protein, 650 aa, synthetic construct
MXXXXXXXXXXXANXXFXXXXXXXAXXXYXXXIXXXPXLXEXXXXNLXLXXXXXXXXXEXXXXXJXXXXXXXXXIFVGIAAIPERXXALXXTIXSLJXQXXXIGVYLNGWKXXPDXLXXEKIXXXGFXXXDLGDVGKFXWVDXHDGJYFXCDDDLXYPXDYXXRTXXKLKEXNYXAAXGWHGSLJXXXFXXYYDXXSRRVFVFSAHRPXDTPVHILGTGCXAFHTXXLXIKKSDFXHPNMADIFFSIKGQEQXIPFIVJXHEKBEITEXXGXKESSIXXHSXXBXXSKKNTXXJQNXFVXXNXPWVXXXXZXXSXLIXGRFEXYSKGGIYKSCHLIKXHLXXLGHBVXIXDTXNXXXXXXXXXXDJCWIYPGDPERPDFSXVXDKIXXLXXXGXPVJXNLSYLYXXXRXXWIXXKJXXXNXXXXTPVLXAVFTEXAANDPLLXXVRDYXCVVPKTJLPTPXEXXXXFXZREGICLGDATKLGNXXVIGGXXNXWIDAIHNRLPHVNJYAYKQYQGNNPHPKXKYXXHMKENFGXWLAQRRJFICXNVHLTFEMVACEAQXYGTPXJYRHMPHSLSEYISATGXAXRXPXEMXEMXAWLYNXXXXWBKXSXXSJXNXXXXXVBLLDXSLEGYJRLAIXRXXXXXXKFLTKK

SEQ ID NO: 9 - DNA, 7080 bp, synthetic construct
  source 1..7080: mol_type = other DNA
tccaaaatttcagagtcaattttcgtaggtattgccgccataccggagcgcgccaaagcactggagaaaacaatcgagtcgctgttaccgcaggttgaaaagatcggtgtgtacttgaacgggtggaaagaggtaccagattatcttaagaatgagaaaattctcgtcgaaggatttggaaaagaggacctgggcgacgtaggcaaattcttctgggttgatcagcatgacgggatttattttagctgcgacgatgatttaatctatccgaaagattatgtggatcgtaccgttgagaaacttaaagagaagaactacaaagcggctatcggctggcatggctcgttgttacgtgataatttctcaacgtattatgataagaatagtcgtcgcgtatttgtatttagtgctcatcgtccatgggatactccagtgcatatcttaggaacagggtgttctgcctttcatacaaaattcttgaaaatcaagaagtctgacttcctgcaccccaatatggcggacattttcttttctattaaggggcaagagcagaaaattccttttatcgttttggcacatgaaaaggatgaaattacggaatttgttggcgcaaaagagtcatcaatctatagtcacagtcaagccaatgtggagagtaagaagaacactcatgatctccagaacggcttcgtgatgaagaatatgccttgggtcatgaatgacgttgaatctctgtcagtcttgatagtcgggcgctttgaaaactattcaaaaggtgggatatacaagtcgtgtcacctcatcaaggaacatctttccgctctgggccacgatgtggatattcatgatacgcagaaccctttcgcaaaagcgcttgagaagaagtatgatctttgctggatataccccggagatccagaacgaccagattttagttcggtggaagataaaatctacgagttaaagagtcgaggaatcccagttatagtgaatctgtcatacctgtattcagaggatcggacaatctggattcgtaacaaaattcgcgatttaaacgccaaaggtaccacccccgttctgggcgcggtgtttacagaaacggctgcaaacgatccgcttcttaaagacgttcgtgactatatttgtgtcgttccaaagacaattctcccgactccttgtgaacgctactacgagttcggcgagagagagggtatttgcttgggagatgctaccaaattaggtaacgcaaaggtcattggcggaaatgtgaataactggattgatgccatccacaatcgcttacctcatgtcaatctgtacgcctataaacagtatcaaggtaacaatcctcaccctaaaattaagtacgccccgcatatgaaggaaaacttcggcgactggttggcgcaacgtcgaatctttatctgtctcaatgtgcacttaacctttgagatggttgcgtgtgaggcgcaatcgtacggaacaccagtgatttatcgtcatatgccacactcactttccgaatatatatccgccacgggtttcgcgactagatcccccgatgaaatggctgaaatggtagcgtggctgtataacaacaataaggcctggaacaaaatgtcccaagcgagtctcaacaatgggaaagccaacaatgtcaatctgctggacagctctttggaagggtatcttcgtctcgccatattgcgtattaagaagatgatggtcaaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatcc

SEQ ID NO: 10 - DNA, 7095 bp, synthetic construct
  source 1..7095: mol_type = other DNA
tccaagatttccgaatctatcttcgttggtatcgcggccatcccagagcgagaaacggctcttaagcaaacgattgcctcacttataaaccaagctccacgcattggtgtatatttgaacggttggaagcgtattcctgacttcctgcgccacgaaaagatcgagatagcgggattccacgacgccgatcttggagacgtcggcaagttccactgggtggacaatcatgacggcctgtactttacttgcgacgacgacctggtatatccccctgactatattaagcgcacagcagctaaactgaaggaacacaactacagagccgccgtcgggtggcacggatctttaatcaaagagccctttgtgaattattacgatggagcgtccagacgtgtgttcgtgttcagcgcgcatcggccagtggacactccggtgcacatactgggaacgggttgttgtgcgttccacacgagagaattaagcataaagaaaagtgattttgtccatccgaatatggctgacatattcttctcaatcaagggtcaagagcagaacattccttttatcgtgatcaagcacgagaagaacgagatcacagaggtagaaggctcgaaggagtcaagtatatttgcacacagccatacagatcaagcctctaagaagaacaccaaggagatacaaaatacttttgtctcaaataacttaccatgggtactgaagtcctttcaacccttcagcatattaattatcggtcgctttgagagctacagcaagggtggaatatacaagtcctgtcatttaattaagaaacacctcacggagctgggacataatgtttccatcttagatactgggaacgagatgacggctgagcatgtgagcgggcacgatatctgttggatataccccggtgaccctgaacgcccggacttcagcactgtagacgacaagattcacttgcttcgcaagcacggtgtgcctgtattgcttaacttatcctacttatatgaatcacacagatcccagtggatagtagaaaagttgaaggagtacaacagccaagcgggtacaccagtattagcagctgtgttcaccgaaagcgcagcaaatgacccgttgcttaatagtgttcgcgactacgtatgcgttgttcctaagaccttgctcccaactccatacgaaatggttccctctttcgaccaacgtgaaggaatctgtttaggagatgccactaagttaggcaatcctatagtaatcggtgggagtataaacccgtggatcgacgctattcacaaccgtctgccacacgtaaacatctacgcctacaagcaatatcaaggcaacaacccgcatccaaaggtcaagtacgtgagtcatatgaaggagaattttggcgagtggctggcccaacggcgtctgttcatctgcgccaatgtccacctcacattcgagatggttgcatgtgaggcacagcagtacggtaccccaacactgtaccgccatatgccccactcattatccgagtatatctcagcaactggcatggccgtacgtacccccggtgaaatgggcgaaatggcggcgtggctgtacaactcacgggccacctgggacaagttatcagcatcatctatacacaacgccaatagtaaacacgtggatcttttggacgctagccttgaaggatacataagactcgctatttaccgtgcatctctcctgaagagtaaattcttgactaagaagtaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatcc

SEQ ID NO: 11 - DNA, 1456 bp, Bacillus megaterium
  source 1..1456: mol_type = other DNA
atgtcaggcatgttcaaggtcgctaacgacttcttctctaatggaaacttcgaaaaagcgatagagcgatatgaagaaataatatttaagtacccaggtcttacggagtttgcttcaggtaacttggcgcttgcacgtcgcaaacttggggaaagacaggaaaacaagagtaagtcattggtgaacgccagcaagatctcagagagcatcttcgtggggatagccgctataccagagcgtgcgaaagcgcttgaaaagactattgaaagcttacttccgcaggtggagaagataggtgtctacttaaatgggtggaaggaagtaccagactaccttaagaatgagaaaatccttgttgaagggtttggtaaagaagatttgggagatgtcggtaaatttttctgggtggatcagcatgacggaatctactttagctgcgacgacgaccttatctatccaaaggattacgtggatcgaaccgtagaaaagcttaaagagaaaaattacaaagctgccattggctggcatggtagcttattgcgcgacaatttctcaacctactatgacaagaacagtcgtagagtcttcgtgttttctgctcacagaccttgggatactcctgtccacatattaggcacaggatgttctgcttttcatactaaatttttaaagatcaaaaagagtgactttctacacccgaacatggcagacatctttttcagtatcaaaggacaggaacagaaaatcccattcattgttttggcccacgaaaaggatgagattactgaattcgtcggtgccaaagaaagtagtatctactcacacagtcaggctaacgtagagagtaaaaaaaatactcacgatctacaaaatgggtttgtgatgaaaaatatgccttgggttatgaatgacgtagaatcactaagtgttttaattgtcggacgtttcgaaaactattctaagggtggtatatacaagtcatgtcacttgattaaggagcacttgtcagcgttgggacatgatgtcgacatccatgacacacaaaatccgttcgcgaaggctttggaaaaaaaatatgatctatgctggatttatccaggtgatccagagcgtccggacttcagctctgtggaggataagatctatgagctaaagagtcgcggcataccagttattgttaacctttcatacttgtatagcgaggaccgaaccatatggatacgcaataagattagagaccttaacgcaaaagggacaactccagtgcttggggcagtctttaccgagactgctgctaatgaccctttgctaaaagatgtgcgcgattacatttgtgtcgttcctaaaaccattctaccaaccccatgcgagcgatattatgagttcggtgagagagaaggtatttgtttaggcgacgccacgaagcttggaaatgcgaaggttattgggggcaatg

SEQ ID NO: 12 - DNA, 1461 bp, Bacillus subtilis
  source 1..1461: mol_type = other DNA
atgtcaggcatgtttaaggtagccaacgacttcttctcaaacgggaacttcgagaaagcaattgaacgttatgaggaaatcatttttaagtacccgggactgacagaattcgcgtctggaaacttggctttagcacgtcggaaacttggtgagcgtcaggaaaataaatctaagagcctggttaacgctagtaaaatctctgaaagtatctttgtcggcatagcggcgataccggagcgggcgaaagccctcgaaaaaacgatcgagagtctcttaccgcaagtcgaaaaaatcggcgtgtatttgaatgggtggaaagaagtaccagactatttaaaaaatgagaagatattggtggaaggctttggaaaagaggaccttggggacgttggcaaattcttttgggtggaccagcatgatgggatctacttctcttgcgacgacgacttaatctacccaaaggattacgttgatcgcactgtagaaaaattaaaggaaaagaactacaaagccgccattggatggcacggttccttgcttagagataattttagtacctattatgacaagaactcccgccgtgtctttgtcttttcagcgcaccgtccgtgggacacgcctgtgcatatacttggtaccggctgtagcgcgtttcacacgaagttcctcaagatcaaaaaatctgactttcttcatcctaatatggcggatatctttttttcaatcaagggccaggagcaaaaaattccgttcattgtgttggcgcatgagaaagatgagataactgagttcgtcggcgcaaaagaaagctcaatatactcacatagccaggctaatgtggaatcaaaaaagaatacgcacgacctccagaatgggtttgtgatgaaaaacatgccatgggtaatgaacgacgttgagtcattatccgttttaattgttggtcgtttcgaaaattattcaaaaggaggaatatacaaatcctgccacctgatcaaagagcacttgagcgcattgggtcacgatgttgatatacacgatactcagaatcctttcgctaaggccctggagaaaaaatacgacctgtgttggatctacccgggtgatccagaacggccagatttcagtagcgttgaggacaagatttatgaactgaaaagtcgggggatcccggtaattgtaaacctttcatatttatacagtgaggaccgtactatatggattcggaacaagatccgggatcttaacgcaaagggcacgactccggtgttgggggcggtattcaccgagactgcggctaacgatccattgttaaaagatgtccgggattacatatgtgttgttcctaaaacgattctgccgacaccttgtgagcggtactacgaatttggcgaacgtgagggcatatgcctgggtgacgctactaaacttgggaatgcaaaagtaattggtggaaacgttaat

SEQ ID NO: 13 - DNA, 1489 bp, Escherichia coli
  source 1..1489: mol_type = other DNA
atgagcggtatgtttaaagtggcaaacgatttcttttcaaatggcaactttgagaaggcgattgaacggtatgaggaaatcattttcaaatatccaggtctgacggagtttgcctcggggaatttggccctggcgcgtcgtaaacttggcgagagacaagaaaacaaatctaagagcttggttaatgcgagcaagattagcgaatcaatctttgttggaattgccgccataccagagcgggcaaaagccttagaaaagactattgagtcacttttacctcaagtagaaaaaatcggcgtgtatttgaacggttggaaggaagtaccagattaccttaaaaatgagaagatattagtcgaaggcttcggaaaggaggatttgggggatgttggtaaatttttttgggttgatcagcatgacggaatatatttttcgtgcgacgacgacctgatttacccaaaagactatgttgaccggactgttgaaaagttaaaagaaaagaattataaggcggcgataggttggcacggatcactgcttcgcgacaacttttcgacatactacgataaaaattcacggagagtctttgtattcagcgctcatagaccatgggacactcccgtccacattcttggaacggggtgtagcgccttccacacgaaattcttgaaaatcaaaaaaagcgactttcttcaccctaatatggcggatatcttcttttcaattaagggtcaggaacaaaagatccctttcattgtgctggcccatgagaaggacgaaatcacggaattcgtcggggccaaggaatcgtccatatatagccactctcaggctaacgttgagtctaagaaaaatacacatgatttgcagaacggattcgttatgaagaatatgccttgggtaatgaatgacgtcgagtcgttaagtgttttaattgttggcagatttgaaaactatagcaagggtggaatctataaatcgtgtcatctgattaaggaacatttatccgctctgggacatgacgttgacatacacgacactcagaatccatttgccaaggccttggagaaaaagtatgatttgtgctggatctaccctggagatccggaacgccccgattttagttcagtcgaggacaagatctacgagcttaagtcgcgtggtattcctgtcatagtaaatttatcgtacctgtattctgaggatcgcaccatttggattcggaacaaaattcgcgatcttaacgccaagggaacgacgcctgtcttgggagcggtcttcacggagactgcggcgaacgatcccctgttaaaggatgtaagagattatatctgtgttgtccctaagacgattttaccgaccccctgtgaacggtactacgagtttggggaaagagagggaatatgccttggtgatgccaccaaattgggcaatgccaaagtaatcggcgggaacgttaataactggatagacgcgattcacaatcggt

SEQ ID NO: 14 - protein, 662 aa, synthetic construct
MXXXXXXXXXXXXXXXXXXXAXXXFXXXXXXXAXXXYXXXJXXXPXXXXXXXXNJXJXXXXXXXXXXXXXXXXXXXXXXXXXXFVGXAAIPXRXXALXXXIXSLLXQXXXIGVYLNGWXXXPXXLXXEKIXXXGXXXXDJGDVGKFXWVDXHDGXYFXCDDDLXYPXDYXXRTXXKLXXXXXXAAXGWHGSXLXXXFXXYYDXXSRXVFXFXXHRPXDTXVHILGTGCXAFHTXXXXXXKXDFXXPNMADIFFXJXGQXQXIPFXVIXHEKXXIXEXXGXKESSIXXXSXXNXXXXKNTXXLQNXXVXXNXPWXXXXXQXXSXLIXGRFXXXXKGGIYKSCHLIXXHLXXLGHXVXXXDTXXXXXXXXXXXXDLCWIYPGDPERPDFXXVXXKIXXLXXXGXPVLXNLSYLYXXXRXXWIXXXIXXXNXXXXTPVLXAVFTEXAAXDPLLXXVRDYXCVVPKTIXPXXXXXXXXFXEREGXCLGDATKLXNXXXIGGXXXXWIDAIXXXXPXVNLYAXKQYQGBNPHXXXXYXXHMKEXFGXWLXQRRLFJCXNXHXTFEMVXCEAQXXGTPXLYRHMPHSLSEYISXTGXAXRXPXEMXEMXXWLYNXXXXWXXXSXXSLXNXXXXXVDXLDXSLEGYLRLAXXRXXXXXXXXXXXXXXXX

SEQ ID NO: 15 - protein, 572 aa, synthetic construct
SIFVGIAAIPERXXALXXTIXSLJXQXXXIGVYLNGWKXXPDXLXXEKIXXXGFXXXDLGDVGKFXWVDXHDGJYFXCDDDLXYPXDYXXRTXXKLKEXNYXAAXGWHGSLJXXXFXXYYDXXSRRVFVFSAHRPXDTPVHILGTGCXAFHTXXLXIKKSDFXHPNMADIFFSIKGQEQXIPFIVJXHEKBEITEXXGXKESSIXXHSXXBXXSKKNTXXJQNXFVXXNXPWVXXXXZXXSXLIXGRFEXYSKGGIYKSCHLIKXHLXXLGHBVXIXDTXNXXXXXXXXXXDJCWIYPGDPERPDFSXVXDKIXXLXXXGXPVJXNLSYLYXXXRXXWIXXKJXXXNXXXXTPVLXAVFTEXAANDPLLXXVRDYXCVVPKTJLPTPXEXXXXFXZREGICLGDATKLGNXXVIGGXXNXWIDAIHNRLPHVNJYAYKQYQGNNPHPKXKYXXHMKENFGXWLAQRRJFICXNVHLTFEMVACEAQXYGTPXJYRHMPHSLSEYISATGXAXRXPXEMXEMXAWLYNXXXXWBKXSXXSJXNXXXXXVBLLDXSLEGYJRLAIXRXXXXXXK

SEQ ID NO: 16 - protein, 662 aa, synthetic construct; identical to SEQ ID NO: 14